SEQUENCE LISTING (81 sequences)

SEQ ID NO: 1 - protein, 798 aa, synthetic construct
  REGION 1..798: note = Cas13bt1-2
MEKEQGLYSIDRYQGAGKWCFAIGANRAWDNYNERPKLFSESLLRYEKATRRDWFDEETRGLIKKSDVRQRLRKIRCYFSHYCHDNTCLGFDPDDDLRKIMEKAYERAIFEQRKHLSTETDIETPALFEPHGRITAAGVVFFCSFFVERRILNRLMGRIPGFKKTEGEYGATRQMFSKYCLRDSYSIRASDSNAVLFRDILGYLSRAPSQYYRHNKDQCDKDGHPERKKDKFINLALRYLESFVPARLRNHTLSVGRKEVVRMETNAVAEGEGEYRPYPPKAKVKVVFTEDDPERPYYITHNTVILQTAKKEEDIHHCKVGVNELKYLVLLCLQGKAEKAVAGIEGYVRRIQGRFADHTNKVARDDDERLVRGLPEFVRVASGIETPDEVRELKSRLDHIRKKWQTKKAESAEAQLHRKARDVLRHINWESQRPLGIEQYNRLLELLVNRDLESFAAEMKELKRRGLISEELLKSVEGIRNLNTLHVKVCNLVLTRLEHLVENDPEELKRHIGIVPREEKEGPSYEEKVRAFVQQPMMYRGFLRNSFFKGSGKSFAKLVEEELHKKGCPDVPLGTDYYLVRDLERDERKNRFHNDNAALYETLALDRLCVLMARDCLVRLNRNLEKHATRISWEATDAGDTICLELPRRDRDHESFRLSFGVRDYPKLYVMDDPVFLCGLMKHFFPDNQAIQYHELYSEGINKYTAMQAEGIAATLKLEEKTIKEKNMQIPATGYIRFCEIVSQSDFAPGEKRVLKNVRNGLLHYHLEFEPTEWAEFREIMKREGFDTAKKRKSTRKK

SEQ ID NO: 2 - protein, 804 aa, synthetic construct
  REGION 1..804: note = Cas13bt1-5
MKIKNENTDKKTELYSIDKYKGRDKWCFAIVLNKAQTNLTENPDLFEQTITKYDRIRKEGWFDEETKKLIYIQENEHKIKGEIKTLAREVLKNLRNYFSHHFYKQDCLIFPKDNIVRIIMGRAYERSEYEIKKNLKEDISIELPALFEPEGKITTAGIVFFTSFFVERRFLHRLMGSVQGFTKTEGEYKITRDVFAKYCLRDSYSVKAQDNDAVMFRDIIGYLSRVPTESFQHIKNPKKQNESQLSERKTDKFISFALKYLKDYGFEDLKGHYTAFFARSEIKKEKEDIEIKDDKKHKPHRMKSKIEIHFDKTKEDRFYIERNNVILKIQRKGGRANILRMGIYELKYLVLLCLSGKAREAINRIDDYLNDLRNKIPHIENMNKEGIGEQIRSLPGFVRSQLGFVQIDDEKKKENRLDYVEKKWEKKRAESKELKLNRKGRDILRYINERCKKPLTIDRYNRILELLVEKNIEGFYHELEELRKTGRIEKNITQALVGEKNINALHIKICKLVQDELKSLEKEDLKKYIGLTPKEEKVVSFEEKLGRILDKPVIYKGFLRYQFFKNDKKSFARLVEEIIKEKTGGLDVPIETEYYSISTLGRFDKANKTLYETLAMDRLCMMMARRYFLSLNKILAKRAQNIEWKKESGKEFIVFKFNMPQDTGKSISIRFSPKDYTKLYVKNDSEFLARLCQYFFPNEKAIDYHKLYSHGINKYTNFQKEGIEAILELEEKIIKKRKINSPENYLSFEEILNQSIYNDEEKNTLIQIRHSLLHYQILFSKNDLTKFYNVMKREGIEKIWSLVI

SEQ ID NO: 3 - protein, 840 aa, synthetic construct
  REGION 1..840: note = Cas13bt1-8
MAVDYSLKQPFYQGVHKSCFTVPLNIAADNCKQKGYRNLLKEAQRSKGGLSDQSIQEAADLIEKRLSAIRNYFSHTYHTDSVLTFQKEDPVKKFLETAWSYAVSETQKDIAESDYTGIVPPLFEDKEGQFQITAAGVIFLMSFFCHRSVLNRMFGSVKGLKRSDREQMGTGEKRDYQFTRKLLSFYSLRDSYAVKAEATRPFREILSYLSCVPHESLVWLSARGKLTEKEKKAFRHFLDPTVPKEALSEESAGDGSDSERPGVRKNNKFLLFAVQFIEAWSRKEKKGLEFARYRKSRVEAPGENQDGSEKRIVRFRSEIRDTQEDWPYYLRNNHALLRLHPGENKEPVDARIGEYELLYLVLAIFDGKGAKAIQKLANYIFEAKKQIQNARVYDRYQDLLPSFLTAGNKPVSAETIRNRLAYIRGELEKMLEAVQKEKKSGRWEMHKGKKIGHILRFLSNSIDDIRRRPNVKEYNRLRDLLQQLQWDEFDKALQSYVNEKLLDETVYRQLRGFHSLDELFERCCRLELKRLEDMEKAGGDRLNRYIGLEPKGKPKNYADLNTLQKKGERFLKGHQLSIPRYFLRNALYKEYQATEERKPTSLYQIVRERLPRTNPILPDRYYLLEEDPKTYSGSDSKIIREMCFTYIEDLLCMRMARWHYEQLSEKLRKKLQWKEVQTGPAGYERFRLIYKISDELSIEFHPSDLTRLDVIEKDDMLTNISQHFLTKKGTVRWTEFVSQGMKHYRDRQKQGIEALFKWEESLRIPEGLWKEEGYLGFEKVLEEAVKHGKIQDKDKEALKRIRNDFFHEHFCGTPADWEVFKRVLKRFLNQGKNEKKRFKK

SEQ ID NO: 4 - protein, 841 aa, synthetic construct
  REGION 1..841: note = Cas13bt1-9
MPVNYSLDQDYYKGTHKSCFTVPLNIAWDNGSKKGCENLLKEAMRTRGGFTQEDIEKVHRSLAEKLNGIRDYFSHYYHEDKPLEFKKGDDDAVKDFLEKTFSYAAGETQKRVKESGYQGIIPPIFELCGDQVRITAAGVIFLASFFVPRSTLERMFGAVQGFKRSDRGDLDTGQKRDYYFTRSLLSFYTLRDSYYLQADETRPFREILSYLSCVPFDSVQWLQAHGKLSKSEEKEFFGRPVEEQDEENPAQTEKQTAPAGRRMRKKNKFILFAVRFIEAWARNEKLSVEFGRYRNIQNEEDRRKQSGKKVREVFFPSALNNLSAEEQDLEGLLYIRNNHALIRIHLKAKTPVTVRISEHELMYLVLAILSGKGGNAVQKLSKYVWDVRMRSRGPLTNMPRNFPAFLRSPASEVSEQAVQNRLNYIRKTLKEIQANLQKEAQTGQWILDKGQKIRHILRFISDSMPDFRRRPSVKEYNELRELLQTLAFDDFYRKLASFQTERKLDAAVWNNLAQCKSINELCERCCQLQQQRLDELEKQGGDELKRYIGLLPKEKGKHYEEQNTPARKFERFIENQLSVPKYFLRCKLFVTGGSRRTNLLKLVQEHLKPKTSVFHEERLYLREEQPGDYPWSDRKIIQKMYYLYVQDLLCMQMAQWHYEHLTPQVKGKIDWEINSESKESDGYNRFKVEYKGPQGCRIIFRVQDFGRLDFLNKAPMLDNICQWFLSGRKEITWPEFLRDGLQRYRQRQILVVRALFRFEENLKIPEEEWKGKSHLSFDEVLERFSGKNRLSEEEKESIRRVRNDFFHEEFEATPSQWRDFERRMSEYLNKEKREKPKKKKR

SEQ ID NO: 5 - protein, 862 aa, synthetic construct
  REGION 1..862: note = Cas13bt1-10
MIENKPISQGSPQGKTVDDYKGEQKWCFAIVLNRACDNYEENHKLFSESLLEFEKTHRKDWLDEETRKLIHNVEEILPPDPQKKYEIKPKNLANIRLNEVRNYFSHFHHKDWCLYFKADDPIRIIMEKAYDKAKEKVIGHLKKEPEIKIPESLFESNGRITPAGIIFLASFFVERRFLSRLMGYVGGFKESEGEYSITRDIFSTYCLKDSYSIHTPDSKAMLFRDILGYLSLVPSEYYPTYLSQIPKRKPDEKLPKDEKYGERKPDKFILFALKYLEEIVSKGLADTYKVSVARMEIIREETKEAEKSDEQYKPRPNEGTVKIVFESKSKPDGEELPYYINHNTVILRIQKKGDKIHFCKMGVNELKYFILLCLQGKTAEAVAAVDNYIHSLQSRFANPAETVRSDEAGIPEFILRQSGKVQDKDKEKAARIKYIRDKWEKKKAESAEMELHRKGRDILRYVNWNSKQPLGTNKYNLLLELLVKKDFDGFGKQLFNLKLKEHISDEVFKRLTAFKTINTLHEKVCSFVLEELTFLEQNEPAKLEEYIGLVRKPAPENNPPPEYKDKVKAFVEQPMIYKGFLRENVFKENKKTFAKLVEETLGRLKYPDVPLGKDFYYVVDPKLSEKENRFQKDNKILYETLALDRLCAMMARICYENINENLRKSGQEIIWKKENDKEFLYLSINPAKLTTATLREPKTSRTAFGDNLKIPLPSGTQNTFTIRFDRKDYTKLYVMDDAEFLGGLVLHFFSKEKEPIDYHRLYSEGINYYTELQRQGIMAILKMEEKIVMNKKIPMTGNYIGFKTIMKESGYPPLEQNTLNKVRNALLHYHLKFEPTDYNKVVEIMKREGLESKNKIRKTDKK

SEQ ID NO: 6 - protein, 868 aa, synthetic construct
  REGION 1..868: note = Cas13bt1-11
MGNISGEKIGIKMDNKKKGNNYSIENYKEDRFLFTAALNIAYDNCKQKGCLNILAECQHSKGGISDEQIKNVKDGIESRLRDIRNYFSHYYHNENCLMFEKDDPIKVFMEATFDKAVSNLSGSTKESDYKGIEPEQLRLFEEYDKKYRITMPGVVFLASFFCHRSNVNRMMGAIKGLKRADRAEMDDGTKRDYNFTRRLLSYYSLRDSYAVKNEETRPFREILGYLSLVPHEAVDWLDSRGELSNEEKKEFLKEAKNQESKEDNDSTDEKTRRGLRKGNKFMRFAIMFTEDWSKKENLEVTFARYEKQEVHLENKKQDGKKERNIKFPHEISASDDDWPYYIRNNHAIIRIKLKDKDAVSARISENELKYLVLLIFENKGKEAIQKLGDYIFDMSQKIRYDNYEPKDARRIPSFLKITRKEPTYEEVNNRLTHIRRELGKIIETIEKELKESKWLIYKGKKITIILKFLSSSIADIKKRFNVEQHDALRDMLQKLKFDEFYKRLSSYVGDGTLDKKTYESIQGIKDISQLCKKACELRLARLDELEKNGGSVLYRYIGLEAEEKNKEYEKLNTNQAKAERFLESQFSTGKDFLRESFYEQEREQKKSLIKIVKEQFANVVPMNEERWYLMNKNPKKFKDKDNKAIKALCNTYVQDILCMKIARWYYEGLSHAYKDKIEWDSTVETGGCGYTRFRLNYKTDCGVVIEFKPSDFTRLDIIEKPKMVENICRSFITSNNDKKRTISWYDFNKEGVTKYRKQQVKAIERIFAFEKGLKIQDEKWQVQGYVPFIKRPEYENKGFKTFILEDAIQQSKIAEADKETLNKVRKDYFHEQFFSSDEDRKVFEKCMPVVDDKKKFGKKNNRMYGKKG

SEQ ID NO: 7 - protein, 871 aa, synthetic construct
  REGION 1..871: note = Cas13bt1-14
MIENKPISQGSPQGKTVDDYKGEQKWCFAIVLNRACDNYEENHKLFSESLLEFEKTHRKDWLDEETRKLIHNVEEILPPDPQKKYEIKPKNLANTRLNEVRNYFSHFHHKDWCLYFKADDPIRIIMEKAYDKAKEKVIGHLKKEPEIKIPESLFESNGRITPAGIIFLASFFVERRFLSRLMGYVGGFKESEGEYSITRDIFSTYCLKDSYSIHTPDSKAMLFRDILGYLSLVPSEYYPTYLSQIPKRKPDEKLPKDEKYGERKPDKFILFALKYLEEIVSKGLADTYKVSVARMEIIREETKEAEKSDEQYKPRPNEGTVKIVFESKFKPDGEELPYYINHNTVILRIQKKGDKIHFCKMGVNELKYFVLLCLQGKTAEAVAAVDNYIHSLQSRFANPAETVRSDEAGIPEFILRQSGKVQDKDKEKAARIKYIRDKWEKKKAESAEMELHRKGRDILRYVNWNSKQPLGTNQYNLLLELLVKKDFDGFGKKLFNLKLKEHISDEVFKRLTDFKTIDTLHEKVCNLVLEELTFLEQNEPSKLEEYIGLVRKPAPENNPPPEYKDKVEAFVKQPMIYKGFLRENVFKENKKTFAKLVEETLGRLKYPDVPLGKDFYYVVDPKLSEKENRFQKDNKILYETLALDRLCVMMARVCFQQINENLVQRAEQIDWKKENGKEFIYLSINPAKLVIAQTQEPKTSRTAFGDNLKIPLPSGTQNTFTIRFDRKDYTKLYVMDDAEFLNGLMQYFFPKEKTIDYHKLYSEGINHYTELQRQGITAILVLEKKIIDREKLPTDVKYIDFRTIMENSGYKREEQIALGQVRNALLHYHLGVEPKENNKGYKGFKPDDFKTFVAVMAREGIRKREKWNLKI

SEQ ID NO: 8 - protein, 873 aa, synthetic construct
  REGION 1..873: note = Cas13bt1-15
MGIDYSLTSDCYRGINKSCFAVALNIAYDNCDHKGCRTLLSEVLRSKGGISDEQIKSQVVDGIQKRLKDIRNYFSHYYHAEDCLRFGDQDAVKVFLEEIYKNAESKTVGATKESDYKGVVPPLFELHNGTYMITAAGVIFLASFFCHRSNVYRMLGAVKGFKHTGKEQLSDGQKRDYGFTRRLLAYYALRDSYSVGAEDKTRCFREILSYLSRVPQLAVDWLNEQQLLTPEEKEAFLNQPAEDEGGDISDSSSSDKNKKSKEKRRSLRRDEKFILFAIQFIEGWAAEQGLDVTFARYQKTVEKAENKNQDGKQARAVQLKYRNQGLNPDFNNEWMYYIQNEHAIIQIKLNNKKAVAARISENELKYLVLLIFEEKGNDAVQKLNCYIYSMSQKIEGEWKHRPEDERWMPSFTKRADRTVTPEAVQSRLSYIRKQLQETIEKIGQEEPRNNKWLIYKGKKISMILKFISDSIRDIQRRPNVKQYHILRDALQRLDFDGFYKELQNYVNDGRIAVSLYDQIKGVNDISGLCKKVCELTLERLAGLEAKNGSELRRYIGLEAQEKHPKYGEWNTLQEKAKRFLESQFSIGKNFLRKMFYGDCCQKRCFDEEKGYNTQAKERKSLYSIVKEKLKDIKPIHDDRWYLIDRNPKNYDNKHSRIIRQMCNTYIQDVLCMKMAMWHYEKLISATEFRNKLEWNCIGQGNMGYERYSLWYKTGCGVVIQFTPADFLRLDIIEKPAMIENICQCFVLGNKKLNSGAEKKITWDKFNKDGIAKYRKRQAEAVRAIFAFEEGLKIQEDKWSHERYFPFCNILDEAVKQGKIKDTGKDKEALNRGRNDFFHEEFKSTEDQQAIFQKYFPIVERKDDTKKRRDKKQK

SEQ ID NO: 9 - protein, 878 aa, synthetic construct
  REGION 1..878: note = Cas13bt1-16
MAVNYSLREKWYRGVNKCCFTVALNIAVDNCKSKGCETLLKEAEHSKGGITDEQIQQSYTEVEKRLNDIRNYFSHFYHGDECLIFKKDDIVKRFMESVFATAVSNVVGGTKESDYKGVVPPLFEQSNEDYMITAAGVIFLASFFCHRSNVYRMLGAVKGFKHTGKEELSDGQKRDHGFTHRLLAHYSLRDSYSVKIEETKSFRDLLGYLSRVPQQAVDWLNERNELSEDEKKEFLNQKSSEEESPEQPEPENAEWRTEKTSRRSLRKTEKFILFAAKFIEDRAEKEKQDVTFARYQKTVTKEENKNQDGKQARVVRLKYEEDKKDDEKPREHFNLEWMYYIRNEHAIIQIKPKDKEAVAARISENELKYLVLLIFEGKGGDAFNKLSDYIFRMTQKIKSGQINPNEARLPSFLKNPVKNITDKMVRNRLDYIRGQIKDVLEKINMEEPQNNKWLIYKGKKISLVLKFISDGISDIKKRPNVKEYDTLRDTLQKLDFNRFYERLKSYVSDGRLAAALYDKIKGIDDISELCKKVCELMFARLAELEKKGGFELYRYIGMEVQEKDEKYDEWNSPQKKAERFLESQFSIGENFLRESFYSEYCQKQECIDKEISLNTSVKNRKSLVYIVKEKLKDIMPLHNDRWYLIDRNPKDFERKDSKVIKGLCNTYVQDVLCMKMARWYYGQLNPALKNNIKWDETGQGHGYDRYKLSYRTNFGITIEFKLADFTRLDIIEKSDMIENICRSFIKPNRTISWYDFKQDGVEEYRKRQYKAVRAVFAFEESLIIPGRDWLSQGFVPFIKNEEYVKKGFSLFVLDEAVRQLKIKGSDKDAMRQVRNDFFHEQFQAKDEQWKVFEGYLSCFMIDRPKGEKNKKRYNGNKK

SEQ ID NO: 10 - protein, 793 aa, synthetic construct
  REGION 1..793: note = Cas13bt2-5
MNMDTIELKKEEAAFYFNQAGFNLRAIESNVFDAGKRKTLLENPQVLAKLENFIFNFRDVTKNEKGEIDVLISKLTDLRNYYSHYVHTDNVKVLSKGEGPILARYYQFATEATASTNVKLEIMDKGNKLTDAGVLFFLCMFLKKSQANKLISSISGFKRTDAEGQPRRNLFTYFSVREGYKVVPEMQKHFLLFDLVNHLSNQDEYIEKSQQTYDIGEGLFFHRMASKFLNTSGILRGMKFYAYQSKRLEEKRGKIEPEGDSFVWIEPFQGNSYFEVDGHKGVIGEDELKDLCYALLVAGKNANEAEGKITQFLTKYKKADNSQEIEKDEMLCIDNFPANYFDGPGVGSIKDRVLNRLEREIKSHKDNKADSKAYDKMKEVMDFINNRLPAAEKMKQKDYRRYLKMVRLWNREKGNIEREFKAKEWSKYFPSNFWRANNLEDVYKLARQENARILGNLKAVVEGMSEQEFEKYRQINEAKDLAGLRQLAGSFGVKWEEKDWEEYARQIKERITDRQKLTIMKQRITAELKKRHAIENLNLRITIDSSKSRAAVLNRIALPKGFVKTHILQTPSDKIMKKTREAECKILLSGKYGDLSKRFFDEKNLDKLTQINGLYEKNKIIAFMVVYLMEQLKLGLKGKTKLAELKETRIRYKISDKVTEDIQLSQYPSLVYVIGRKYIDNVDRYKFAGDVRGKPILSKIDLIEKERLEFIRQVLGFEKELFDNTVIDKKNFTDTETHIKFRQIIDELVRKGWDATKLNKLKEARNAALHGEIPEGTSFQEANVLINELKNKK

SEQ ID NO: 11 - protein, 739 aa, synthetic construct
  REGION 1..739: note = Cas13g1
MDICGRDVFLKRYGGACKNQEERKKQQKFFNEIKNFLKTLRNYFSHYLHTKEKVESLLKEEKCREFVSFLRRKALEVWNKRFENSALFSEMQAFSQNGWEEMKRELKESGPFLTDVLLFVSVFVPRGEMMRLLDAFRLSPDREKRECRREILSALCLSESYDFWSVDKKAGIALTILDHFARIMGKRTDKAGKLIMSAPRKPENRKETPEICRRFRPSFFIRMLVGFIEEENVLKGFEFARSYNGRPVYDESKKELYLCRNNTRVRFPDNEGNLLESELGAEALKRIVLLYLKNLKGNDLAPLVRGKIKAIQKTLPQRKNGTNAALLQRVKSRRDELSIRFFRKGLSDHQKAVAVLDFMNLIFPADKKFGKQAYQEALRALTGRYDEEAFLAVLPDIKCSETPFPKRKKPQAWARGAGSLETLHKRAEALFREKAGSLCEETAEEIGRLFHVRALKEKTGEPFERFKKLLTPPPRAFTGNGFGLTKEELLKDIPLEAELYAPGGGPMASAVKKIYDNDRLLLKLARFILEQIREKDKVQVQITEKPDYDYEALVTVGEKFQVIIDSAQARRSHVSLNTERLKNVIQNYWKREETPVPFVVPDMRKREKNTPPRCWTEAEQDMERERRTMVEALLKWEKYLANEFAKENKTATGNIDAIIKLMKPEKGEYVDFPLLLARMNIPEASEYNDFRKWAYHQAPKKAFSDAPGELKERFLKRQAEKDRKRREQKKNSIKKVQKK

SEQ ID NO: 12 - protein, 769 aa, synthetic construct
  REGION 1..769: note = Cas13g2
MNKRAENKINFFKDNFTLGQYAAFALSFNQAGRQLEKLSTTYHFQRIYGRRRDKVPSFKESLKNLRNYFSHCASQWPDEERFKKSVLRNELDFLVLEAIAILEQRNTKLHDNDETAKDIKKELDKIRSSPSDFFPLDNSGKNIQPTLALVLSLFLTKNQMAFLLGKIFRGNGVTRESPQYLAQAKILETLSQNDRTLVDTDSQSERFTSHDKEFGLAIAGRLEAVGLYEEQQKIEDFPEDVWFIKQLVLYLEYMNVLPSVQFCRMTTIEKDDQLLQEKDFDIAHREKPLRIRCNTVEAQVKLSNGAPYTTNFGVQSLKYLVLAHLKKTFEDKEIDQLVVRQIESNPSRKCRKAKESGVTGDRLTKRIEYLIGKHTPSGENPVRLYEQIRFICRFVNEAWFKRYNRHMNSEEFKDIQERVRHYRRDDFQKLLKESDLLDIAGLNLGAGNDKKLGSCFRHERIQNIFCEMESGYLEWLENRKQQIDKLSQEEREALAARINLRHREGRSSQEAFRPVSISADVLRREIESRDKAKNKRRFFDHIRDFGGSARFFARFDLDDRGWGSARDRERWARTGLLSQIVLKSLSGANIKNMLDQKPSQWECEERVDGTKITFKLTQGWRYYAATTKTQLRKLIGAYRPDLSVLPLLDDGGIEGGSVQSLKRTLERERYRLMQAILVWEREIIEKHDMKPDGGYIKFESILEKGATAEQAPDLKDIRNHCMHGDIWKAPFSQAPEPLRRVYSGLEEKSREKRRSQKKSGIKRSQKNQK

SEQ ID NO: 13 - protein, 767 aa, synthetic construct
  REGION 1..767: note = Cas13g3
MRGGNKSANEGKKFDFNLKQQAIFALGSNWAQKQFELTKKTQHSHRLREKLSIFQEGDIGRLEEKIENLRNYVSHGAHSGLAPLDSDEIAAFEAIVRKAVTSYLAIPREAQNIKEEKQTKIDKIRARMKKDKPLLSFSTGPLHQQNHPELMFLLAHFLTRKQLSYLIHRVYWPKEREDDKEPIQELLLFIAQPDTIIMRSKADEDARDTWISAEEEQGFAIWNYLQKRHADEVYTAPDDHYVMRQLVAFIESHQILKGAIFMRVEVRPDEKKEGAYKRVGVYEKQGSDNSLPLNIAYNTIRVSFVEDKVEGTFSLKTLIYITALFIGRVTPDKLTDFLITELKKNRDYSHRPSPAAKEGGDIKTRVEKRLCYLLRRLNKPPQNLQEQIRFICQRINFAYQQKYGQYLDQNDYKTLENLVRYYRKPDLLSWLEGNAIGQQSGIHMGQEDSKTLNQLIKASSIEQLYLDMKSHYRYGLKYVAKNYQNWPEDKVADLANIIGVRQQKTVSGNLPNAPVGVKLSWILTEFQDEIEEFKGIIHMLSAQYAPFKFEKPDKKFKKPGDAPRSEKNKRGWAHAKPFVVKQLLLNMAWYNVKEISDNDARLAGGAFVPISDISFKRSFSGCSLRMSFGKSWRQHARKSREYLQGLIDSYGEGRREFVLSKAEEENGISKSIERMEEEARQERLLFIQAILDWEKGWLKKNKSEAERLKTAKGYVEFREIAEKESLPDTIKELRNKAFHDGFLRDTKFSDCVEPIKSIYEDLKQKHI

SEQ ID NO: 14 - protein, 783 aa, synthetic construct
  REGION 1..783: note = Cas13g4
MSEDLYRSDNLFCLSLEQQAALSIALNVALRRCREQNDSDMDICGRDVFLKRYGGACKNQEECKKQLDFINEIKGFLKTLRNYFSHYLHTKKKVESLLEKEKFRDFVSFLRLKALEIWNKRFGKSSLHSQMQAFSQNGWEEMERELKERGPFLTDVLLFVSVFVPRGEMMRLLDAFRLSPDRKKRELRREILSALCLPESYDFWSVSKKAGIALSILDHFARIMGKRTDKAGKLIMSAPRKPENRKETPEICRRFRPSFFIRMLVGFIEEENVLEGFEFARSYNGRPVYDESKKELYICRNNTRVRFPDNEGKLSESELGAEALKQIVLLYLKRKQGAFKGNDLAPLVRGKIKAIQKTLPQRKNGTNAALLQRVKSRRDELSIRFFRKGLSDHQKAVAVLDFMNLIFPADKKFGKQAYQEALRALTGRYDEEAFLAVLPDIKCSETPFPKRKKPQAWARGAGSLETLHKRAEALFREKAGSLCEETAEEIGRLFHVRALKEKTGEPFERFKKLLTPPPRAFTGNGFGLTKEELLKDIPLEAELYAPGGGPMASAVKKIYDNDRLLLKLARFILEQIREKDKVQVQITEKPDYDYEALVTVGEKFQVIIDSAQARRSHVSLNTERLKNVIQNYWKREETPVPFVVPDMRKREKNDPPRCWTEAEQDMERERRTMVETLLEWEKYLANEFAKENKTATGNIDAIIELMKPKKEGYVDFLSLLERMKIPEASEYNNFRNWAYHQAPGEAFSDAPGELKVRFLKRQAKKDRKRREQKKNSIKKVQKK

SEQ ID NO: 15 - protein, 801 aa, synthetic construct
  REGION 1..801: note = Cas13g5
MIDSFKDGFTLKQFVAFTLAANWAFDQHNNDQHNNLINTQHFYNLVKKNMKFKGDEDIFRGEEEAESDAPPKSIIKSIRNYFSHFIHSPLRSLTTVEARDLNDMARRLLVELNERNIKANDKDDIQQHIETYLDESTSLFTFEPKPILDHAQPEMAFMLALFLSKGQMAFLCGKIFFGTDARSKQKDGTYKDTPKTCLQKKLLNMMAQPDNIIRRLQSDDTLDPWLESKHEQGFAIWQLLASSQKETAGGNEPDYLKNGNYMIRQLVRFIELHNVLPSYEFARIETVETLSDDVKKLEQKIQFSSNPDLPLATRHNTIQSVNQQGIKGTFGVRTLIYIVVVFLEQKRAGELANFVTTWLKANAHYPKTNLKGKDRPLSDQIAKRLDYLLKETNVKSKTNAKKSLHMQIRFICQRINHVWSLKYERHLSVHEYGELEKMVRYYRKAELRGWLADHGLLDIQNIQLGRGGIKTLKKAIKAESIQQLYVDMLSDYHCWLQEQKDKLPKLNDAAQQSMAKAINVRNNRQSNNKPDFPIGLPEKVLRQKFFVVDGKSHARSLTTILKSIPTPVTFNEPQGTKSKALKKSMQIRNYQQLLLAMAWHAVKDLISDERIKQEKNSETLPDLAEIPISINCENVRISMKFKKSWRNMATLNKGYIGKLMKQYCAGEKTIPMFRADETKAGKSVETTMQTFHSERYRFMQALMQWEKEFYNTNPALAESSDEIYLKFECLAEKANLADGIVSMRGDAFHDGVPNARFADCPEPTIKTIYDQICEREKDKQKTQRQQGVKRNLAAKKNADKS

SEQ ID NO: 16 - protein, 852 aa, synthetic construct
  REGION 1..852: note = Cas13g6
MNHDVAHSHIKDDVTLRQAAAFTLASNWAHQQHKGVLASVHGESLYRYALPVDEETIAAYRNHFAHLHRPHMPRLWQEPDVKDRLHTLVKEAARMMTMREGKIWDDDEKQHNAIHTILLRIENDPAYGFDLDRPQQAWLSITLILAPFLQKGQMAFLWDKMVEKRGDADEALDKARFHLLKFLSIADSALAITPQQEEERLFSAQTEHGLALLVRLKESLDKITQDESSQATHAFPQGGYVMRQLILFLERTNALPSVMFARTRTHLEGERDGKPLLRQERIFVRERDAQTHDGMPLPPLKIQANTIRVQIQEPKTDKQWQGMLGIHTLSVLVCAFLLGKDVDRLVLSWYREHGDKNDWRQGKEAKSPTPERIGKRIDWHRERMEGATTLYQKIRMLADLMHESYREKHGVAMSTSDYRDLLYHMRHYRLHTLKAMARQDYGIETMNVPMTGKPWSFLLKKQDLDAQYDDIVAYRCRWLSDVKERCHRMDDKERMTLARALGVRDVSPKDVNARGKNRVALLPAGMPVDAVRDAFPEFRAGHPDDKHAKKRHFIDVMRDNKTLKQGGGIPTQPFGFTWDTDKANKDKADKDKTCAGGWRAMTKRRSQWAHASLLHMMARHLLQGDYAVETPHDKKGKATSHESNKKKQVALGEMPFASVKPSALGMTLEVEQGKKVLCSLTQGWRYYARLDKKTIKNLVTAYTKAGKGRTWTLPLLRASAGDKEESVEQAVTEMRRQRFIAAQAILQWEYDVLKGKDIDKDKRLDFPAILTYAGLHDDSIRKKLTHYRDYVFHDNVLEHPFRDAPDILRECYKKIEAEEKKRRQQKRRQHIHQRPVQHSGKGKGSKKHKKTR

SEQ ID NO: 17 - protein, 867 aa, synthetic construct
  REGION 1..867: note = Cas13g7
MQDRQVKREPFKDGFYLKEYAAFAIAAHNAHLACIKCATSILETEFFAKRYFPLLKAAIAHHPDFTPNAGQASWPQREELASFTKSDRAQKYNQAFSKCLTEYAGGIRNYFAHYLHTLKPLQLPAEDRVFCFFLEELQKKAVAVWKARHAAWRAKQSPHGEQYDSILECSQRPDFWDPLTVQRDDCTILSCEAILIFLSILLPRSRMYMLLDKVLVRGESGTETEKKRISKRELLTALSPRDGHSIRIDPERKSLTAAFTVFNHIGTRYGRLTDTDGNLLSPRPNNIDDIVASHHTVFFIKMLVAFIEGEKCLPSFEFARSYHGKPVFDQPDRELYIRHNNVAVRLRDEPRQQAVFSIHLLKLIILNLLSDKGNQTPDVDDWLKKQLRNFRSRAGQGDHSATPNSNLERMTPSFMKGEPGRPPKQACSLRKIVENRLQLLQERWFGENAETLLPHQMAYAILRAVNLMVPPENVLNSLQFREALLALTAIPYSADAVKGALPDIPRRKLNVGGQTLRGRLSGSQNINAAYGKIRSGFSEYCNEMMDLFDDQETTALRTFAARIGVQYIRKENSLEHERHKKDREQYQKHYLEKLVSIPPVLFVRQFMDDDAKNVCFGTLLQRSRYIREVREAGLWDVKTLLADGQKFRDKHKEVINRDQLLLALVQYVQERNLKAQIKTGKKKKQGNITSLFDLSEWKSSFAFRVEGGKSVVVDTAKAWKYYLRTDPAWLQKVFKTYCSEIQTDTLPFVGPPDLPRRKENTPPRSWREAEGDMEQERYILMRSLLIWEKNTGSSRAKGEEYKPFKDLVTEAALPNGDEVTSIRNAAFHGVPDKPFRDAPEPLQQIYQQERERLQKKRGEKKRKAQKN

SEQ ID NO: 18 - protein, 1140 aa, synthetic construct
  REGION 1..1140: note = Cas13h1
MENTSLRTFKRFFDFKGSVAPIAEKANRNYALKKRNNVNLQQRLHYFAVGHAFKNIDVENIFRAELDEETKGKKPTKFLALQLSNFTFIKELGCLLSNIRNINSHFIHDFELIKLDKIDDKIIEFLKQSFYLAVIQTCIKEKETTYIDFISTKDYEKQIVNFLLEKFYPFNDKRKKLTEEEEKRVKEYKSFRNDFKNKSIDEAIDSILFVKVNETVEWNLFETHNVFNITSGKYLSFEACLFLLTMFLYKGEANQLISKIKGFKRNDDDKYRSKRNLFSFFSKKFSSQDIDSEENHLVKFRDLIQYLNHYPTPWNKDLELESANPAMTNKLKEKIIEMEIYRSFPDFANDERFFVFAKYQIFGKKYLGENIEKEYMDNSFTAAEKIAYEYEIKISPEIKDANNKLKELKAKQGPYGKQKERNEKIIRELESMIKDGKNDPNPITEKLKTRIVKNLLYVSYGRNQDRFMDFATRFLAEEKYFGEDAEFKMYKFFSSEEQENNISTLKKELSKKQYDNLKFHQGKLVHFCTFDEHLKNYESWDDPFVIENNAVQVKVYLSNGINKIVSIQQNLMIYFLEDALYCNENVKELGKTLLTDYYLMHKEEFEHTKLFLQQNPTISREDKTVFKKILPKRLLHHYSPAMQNNLPEFSTFQLLFEKAKRLEERYNKLKNKAEDEGNLDDFLKRNKGRQFKLQFIRKACHLMYFKESYLRQVQEAGHHKRFHISKDEFNDFSKWMYAFDETPKYKDYLRELFSQKGFFDNPAYKKLFEDSVSLDTMYLQTKKNYEEWLKTFVPGQRATDKYSLNNYVKFFDNDLFFINISHFIHFLESRNKLQREENGNIIYHSLSNSEYLINEYYFKDKLEKSEYKTCGKLYNKLKTIKLEDSLLYEMSMHYLQIDKSIVKNARTSIINLLVQDVKFSIEDANKNHLYDLFIPFNKIDSFVELIKHKEEQEQDKRFGGNSFLSKLSSYIEKVRNNKDIKPIFDRFIKKNSLNFDDLNKINNHIITNSAKFTKVELSLEEYFIFKDKILIKKENRINIDEIKNLSKYFNKVDRHNAFHFNVPNECYDIFLNKIESTYVKDEVRPVAPKQYPDLTKQQRSVCSTFLDAIHNDFFNRSDREKMREEAENKYFQKIILNRMN

SEQ ID NO: 19 - protein, 1149 aa, synthetic construct
  REGION 1..1149: note = Cas13i1
MSIKITVGSSNDGFTRHETKWHDSDWDENDDSKVPRCIDIGGSRIGALTEHLEIVVPKDMGTKKKKDEVKDFTLYLNYAVSNLREITGIEGDEESKIKERLENLSDEKKQRLADFLWAFRFDEPQRDFAARGNDQKQFDHDYKRLAPLVATKIFELRNYFAHLDRRGNEALVCDRELYVLLEGILRPLAEKECMGPGCQTSKLYKMHLLNLRGPRKPPQPMESRKYDLTRRGVMFLVCMALYKDDAEEFLSCFADMRVPNRRRDSLDGLSETDIGKLGRKKPSKRAFLKAFTFFSYRRGRVSLDGEDPDFLNFANIIGYLNKVPGASFDYLALEDERKLLADLASRSTESEENKLFKYDLSKQVRAKDRFVSLAAAWCERFDVLPCIHFKRLDITPSLGRHRYLFGKENDNTVHLDRHYIIENGAIRFEWRPTTHYGDIKIGYLRSCIGEAEFRRLLFCALRKPAETNAMLDAYFTAYHKILELMLNAPALDSFSIFSNDELVEAVQTVTGLTADELADIPEERLRQFFPKNLWRFFFAWEASQDDDDLRAAVAAKIDGRIKWCSDFLVRVENFRKWKAENFWKPNDQRGPQGACPKSKLVNPPRNCRTSDASCVARVIAYLNYHLAPERKFRQLSLGKQHSHPGRNDNDTHNYEYQLVQSAIGKYALDQTSAVGGTKNGRVIKGTVAHLRPELELHLAQLRDKVRALARENLQPMEDSLRAAKRKNLLSGFACLDADKALKSARNSRKLIDLAEAAAELLKEELLKEAESLLSVGGDALKVLCRKFGVRTGLPLDKDALVKTILGIDMDKWTHAFDYGHGHPYVDRKLTEGDHVAAQIPLPNGMAERVMRSQSRVFDGLIGESGIDWAKAFASLDEKNISLLGFYDVSPLVGYIKTHSDHTEEEPDSSAPGINTWADADSRTIKPDFSRGGINKAIRAIKTAHNQDRLLLKFAIQHWEEFKKTRAYDERKTAYEKALSVRDYFNGTFTYALKGGVGLRLSRNDVFSPTFTHVVENAAAIVDLLKHENPDVKEVSFYDVAQLFARKQRESRKLRMELLPLIEQFGAKSPIPQEEYTRINAEGGKNDERKKKIRAMEYSYYSKAMPTLTEAEYNLVADVRNAVMHSTLLVTDDDAYRAAKAVLGRLVSAR

SEQ ID NO: 20 - protein, 1111 aa, synthetic construct
  REGION 1..1111: note = Cas13j1
MDTPSKESSGKIIEKLDELKKATQESIEQRKKFDYRIGKGLADKLGNEKDSYFEKYLRKTVGENIPPKILVKPKGGWKTETKGKGKDWGDKITFAAMPQHHYNTLLAMAFTKAHKVYSDIRGKVEIDAAQYETKAKLIEVEYGKDETRGLSGLEYLMMSKHLFSGKNSNIKLAVKKGETEILKEYALNEKLIYTVLDELRNFHSHIFHEPGPVSFKNLYGDEYKPEKKLTEEEWAIARDWFVNRFNDAKEHKLKTLAKVLEREGTTEEKEDAEKVIKTISGYSFEYNNCISREALLFIACMFLRKSDAAYFTKKWTGMKKAEGVFKSTQSFFTDNALKESKSILTLNADLYKYRQILGVLSTMPAMKTDSLKPFYDFIKINNDSYSEKAEKARSKEEKEKIQAFIIPQRKSSNYTYWFMKYLNDNKLLDGFRIAYYKTPEDRFMYLIHNGLISQDDLENIEDFKTPDEKLKYLREKGFNLKLKMKQAVGDEKKSLTEIYKETQRNFVFKVPTIENDNFCVKKLNVFFQTDIEFNGQKISVQLSVSPDFLMKWVFVLLITGEDSIKNAITEKKEKIKDILKKYAEEYYNRCITSNFNEPLMGLEASKVFPSSLTSTVEIDEKIDKDKILMRISEKYNELTKFDEENKSRKAPWRFASKRKIDIILDYVHLVYSDRAFDEKKSVDAMRHEALNDMEYMDTFEYLRYYGRYRETEEFKKIFFEDKKLYFSPILKAMKQLDSLEGVFNFAITGFLNYLKGIQSKVTDENTNKYGKVFKVTGKSLTSKIGHHSEMFSVNHCVPQELIKLNDIKGYMKWKHETKDKLWISDFAFIRNVLESRGGFSNTDYLMKEVMPLITFEKNEKGSIKGNTQMFVALSRNKTNELMLWEIGKYYWKEATGSEFSRLFKGLEKNTGNKITKAYRFTNPYYTIYQEDLDIKIQRKDKKGKVIVNSPVYTIKIKPKKFDDEYQYYEQEHIVDYIENYEPKKGIDGHWHFEELNKKIKDELARYLDDIYLLMTVEKHIVQKDFDKYASLLKEKDSKLPPYYIGFKRNDIALNKVIFDALKHKGSFSADDLNIYRINVLHQILQPKREKYIIIRKSLIEYCAENKLLKTM

SEQ ID NO: 21 - protein, 1085 aa, synthetic construct
  REGION 1..1085: note = Cas13k1
MKKELNREDVFTGGKTPELTIYYNIAYFRMAGLINALCGNKLERDKDALEQFMKAFINGKQKLTDMQFVKLCDYLWKGYKYDKNRSEYSLTENDKTMVLKMVAKLQDIRNFQSHIWHDNKVLVFDADLVQFIKNKHLEAIDAQRQRFSKETEVYEKENRELKKKKNKELLFDIHDGLGYITGEGRNFFLSFFLTRGEMTRFLKQRKGCKRDDTPEYKIKHLVYRHFTHRDGATRTHYGYEDNMLDQLPDKQEFLTTRHTYRLINYLNDIPEEVTNPELFPLFNTISNGILIKESVGTYIAFINKMPLLSDFEFSEVWGKDGKPYTNLVEFYHKTQPGFKFRTDLNSFHKIILNLIRDENYTGFFTTQLNLFMAHREELILVISKPLITPNDLLLIEEHYRYKLKANDFVREKLGEWKEKIEKGKWEKANEQKDKLINLLGGVIEITFYDFYWQKDEKPRNENRFMEFAIRFMADFNLLPDCEWEVEPLQIENDLIANRTATPDLKKSGSQFMNQLTDNYRLKFNDGQIAFRYQNQVFIMGHKAVKNLLIACFDGKEKMLNGFMPALLADLKKITNEHILKNHQALNTLSLLNNDSIPSYISRQWGEAEPITDMKKKAIARMDYITQQFEALIENHHYLNRADKNRQIMRCYKFFEWQYPQNSQFKFLRRNEYHRMSIYHYCLDKEQHKYDKKGHNYLYNRLIKESHNESGNIEQHLPYQIRTMLNDAKDFNDYFLRILNATHKILTDWKNQLKQGREPNNYYLSRLGFTGGLTQKVVHTRLLPFSIHPGIPVSFFYRTEMNQNPSFNLSAKVWNSESPFRVGLKESNYQYAKYLGLFNEIKTQRKIIGKMNQLIAEDALLWQIAKKYLDKCHPKFSEIINQQVNGKQPLNVKAIHDTELTIDFPVDKNLYQVVVKMKQLDDFMFIDSKPVLEKTIRYMIKNSLRDKEYQQRVFIETNPGVFKIPYNEVVEAMRTINHQALTWARLLLDWESKTVKTIPEADKKAKLQEAYNKRQPAFFNFNDICRYADDGKQLKDLRNKTFHTEVPETWTYDEMRENKTIKMVLGINETTFLKKDYAIENARTIE

SEQ ID NO: 22 - protein, 895 aa, synthetic construct
  REGION 1..895: note = Cas13d_896aa
MKKNSNDKTNAKRMGIKSFIKNGDERFITTSIKNEFPVELKLDVIKKTCEPAHEPVSFDYDPKKIDFEKPVLKEKLTSGQSGQKLSTRLFIQKDRDICGIRRKYLEKIFNSNFIEEKKDSNLPMQIVAKVLSTEKVFSNALNKIISQFLSMPRGGVTDNHGEYEIIGNIINHKSLQELNKEKKTKRIKKYLQSVIKNQSYLYNKQFLLSLDESKGSRNDIDENELYDYIRFLAILRNGIAHVFYEKNEPETAKESLFRLVDFIKNDKKLEGAFAKIKIQVNTLYKCRKEEYIKKSGKNFEIIRKIYQNDKPDEKVKDWIRYDFDKSYKYIGLSVAKLGNYTSWAKDIDNLRDKSNPDSGYAGIMHRLNEFSVYLKVKALSTEEKDKYLKNLISKENCEEKDKYYKNIAQFFCSSDLKFANVLQMVKEIKKNKGCTSEDKNCKLCVDERKFNDLSVIVYFISCFLDNKDQNIFLSDLINRFGALSDLLRIQNKILGAGNKYNENYSFLKNERYVTEIKMELETIFALVKVSYKKEDKAFNRLLEDGLVMFGFSKDEAGMKVAGLKEIKEKKEGHYKNKSRSFLINSIVNSRKFAYLAKSIDPQKVPAIIKNEHIVRYILGRINKTNPGQIGRYWRYIMSQNHAGTDKVDDLTNEIIKINIKNILNDAGGWQKSKLNDNNNKKKLKYQQLIGLYLTVAYIFVKNMLECNARYFSAFAQIEKDYLIYTNSDEFYYIDKNKKNLVTERYLKLVKDIIEKNKNTVRKDKIFRKKRQRKHLADISKSIIEFEKLPCCIFTLLRNITEHLNVASNIDIIEGYGKRAGKYHKNAPASYFIFYHYIIQKILADKICTRNLLNIINTYGEPSISFIKIIYVPFAYNLPRYLNLTDARIFCNMDDK

SEQ ID NO: 23 - protein, 910 aa, synthetic construct
  REGION 1..910: note = Cas13d_911aa
MQQNSTDNKNKIKKSAAKAVGVKSLARLSDGSTVFSSFGKGAAAELESLITGGEIRKLSDKAILEITDDTQNKNAYNVKSYRIPNLTARTDKLSDKSGMDDLGFKRELELEVFGQSFDDSIHIQIAHAVFDIQKSLAAVIPNVLYTLNNLDRSYSTDDTAGKKDIIGNTLNYQHSYDNFDKEKLGEFTEYYNAAKDRFSYFPDILCVLEKVNGKDRYQPKSEKDAFNVLSSVNMLRNSLFHFAQKSNDGKARIAVFKNQFDSDFSHITSTVNKIYSAKIAGVNENFLNNEGNNLYIILNATNWDIKKIVPQLYRFSVLKSDKNMGFNMRRLREFAVESKSIDLSRLNDKFLTNNRKKLYKVIDFIIYYHLNKVRKDTFKDDFVAALRASQSEEEKEKLYAQYSERLFADEGLKSAIKKAVDMISDTKSSIFKKKTPLDKALIENIKVNSDASDFCKLIYVFTRFLDGKEINILLNSLIKKFQDIHSFNTTVKKLSENNLIINADYVDDYSLFEQSGTVAKELMLIKSISKMDFGLDNINLSFMYDDALRTLGVSDENLPEVKREYFGKTKNLSAYIRNNVLENRRFKYVIKYIHPSDVQKIACNKAIAGFVLNRMPDTQIKRYYDSLINKGATDMKAQAKALLDCITGISFDAIKDDKHLHKSKEKSPQRSADRERKKAMLTLYYTIVYIFVKQMLHINSLYTIGFFYLERDQRFIYSRAKKENKNSSKNSYLNDFRSVTAYFIPSEIMKRIEKNENKGFLEDFEALWNSCGKTSRLRKEDVLLYARYISPDHALKNYKMILNSYRNKIAHINVIMSAGKYTGGIKRMDSYFAVFQHLVQCDILSNPNNANCFKSESLKPLLLDMRFDGTDEKLYSKRLTRALNIPFGYNVPRYKNLTFEKIYLKSSINE

SEQ ID NO: 24 - protein, 941 aa, synthetic construct
  REGION 1..941: note = Cas13d_942aa
MAKKKRITAKERKQNHRESLMKKADSNAEKEKAKKPVVENKPDTAISKDNTPKPNKEIKKSKAKLAGVKWVIKANDDVAYISSFGKGNNSVLEKRIMGDVSSNVNKDSHMYVNPKYTKKNYEIKNGFSSGSSLVTYPNKPDKNSGMDALCLKPYFEKDFFGHIFTDNMHIQAIYNIFDIEKILAKHITNIIYTVNSFDRNYNQSGNDTIGFDINYRVPYSEYGGGKDSNGEPKNQSKWKKRKNFIKFYNKSKPHLGYYENIFYDHGEPISEEKFYNYLNILNFIRNNTFHYKDDDIELYSENYSEEFVFINCLNKFVKNKFKNVNKNFISNEKNNLYIILNAYGKDTENVEVVKKYSKELYKLSVLKTNKNLGVNVKKLRESAIEYGYCPLPYDKEKEVAKLSSVKHKLYKTYDFVITHYLNSNDKILLEIVEVLRLSKNDDEKENVYKKYAEKLFKADDVINPIKAISKLFAEKGNKLFKEKIIIKKEYIEDVSIDKNIYDFTKVIFFMTCFLDGKEINDLLTNIISKLQVIEDHNNVIKFISHNKDAVYKDYSDKYAIFRNAGKIATELEAIKSIARMENKIENAPQEPLLNDALLALGVSKTDLENTYNKYFDSKEKTDKQSQKVSTFLMNNVINNNRFKYVIKYINPADINGLAKNRYLVKFVLSKIPEEQIDSYYKLFSNEEEPSCEEKIKLLTKKISKLNFQTLFENNKIPNVEKERKKAIITLYFTIVYILVKNLVNINGLYTLALYFVERDRYFYKKICGKALRRKVGDKYDYLLLPEIFSGSKYREETKNLKLPKEKDRDIMKKYLPNDKDREGYNDFFTAYRNNIVHLNIIAKLSELTKNIDKDINSYFDIYHYCTQRVMFDYCKMNNNVVLAKMKDLAHIKSDCDEFSSKHTYPFSSAVLRFMNLPFAYNVPRFKNLSYKKFFDKQWLNH

SEQ ID NO: 25 - protein, 956 aa, synthetic construct
  REGION 1..956: note = Cas13d_957aa
MAKKKRITAKERKQNHRESLMKKADSNAEKEKAKKPVVENKPDTAISKDNTPKPNKEIKKSKAKLAGVKWVIKANDDVAYISSFGKGNNSVLEKRIIGDVSSDVNKDSHMYVNPKYTKKNYEIKNGFSSGSSLTTHPNKPDKNSGMDALCLKIYFEKEIFKDKFNDNMHIQTIYNIFDIEKTLAKHITNIIYAVNSLDRSYIQSGNDTIGFGLNYRIPYAKYGRGKDSNGKPNNSNLKKRESFIKFYNNAKDRFGYFESVFYQNGKPISREKLYIYLNILNFVRNSTFHYNNTSTYLYRKEYKYTDKDNCSVKEFEFVSYLNEFVKNKFKNVNKNFISNEKNNLYIILNAYGEDIEDVEVVKKYSKELYKLSVLKTNKNLGVNVKKLRESAIEYGYCPLPYDKEKEVAKLSSIKHKLYKTYDFVITHYLNSNDKLLLEIVEALRLSKNDDEKENVYKIYAEKIFKAEYVINPIKTISNLFAEKGDKLFNEKVSISEEYVEDIRIDKNIHNFTKVIFFLTCFLDGKEINDLLTNIISKLQVIEDHNNVIKAIANNNDAVYKDYSDKYAVFKNSGKIATKLEAIKSIARMENKINKAFKEPLLKDAMLALGVSPNDLDEKYEKYFKTDVDADKDHQKVSTFLMNNVINNSRFKYVVKYINPADINRLAKNKHLVKFVLDQIPHKQIDSYYNSVCTVEEPSYKGKIQLLTKKITGLNFYSLFENCKIPNVEKEKKKAVITLYFTIIYILVKNLVNINGLYTLALYFVERDGFFYKKICEKKDKKKTNKDVDYLLLPEIFSGSKYREETKNLKLPKEKDREIMKKYLPNDEDRKEYNKFFKQYRNNIVHLNIIANLSKLTSTIDKEINSYFEIFHYCAQRVMFDYCKNNNKVVLAKMKDLAHIKSDCDEFSSKYTYPYSSAVLRFMNLPFAYNVPRFKNLSYQKFFDKQRLEALEKNLNI

SEQ ID NO: 26 - protein, 981 aa, synthetic construct
  REGION 1..981: note = Cas13d_968aa
MGKKIHARDLREQRKNDRTTKFAEQNKKREAQMAVQKKDAAVSAKSVSSVSSKKGNVTKSMAKAAGVKSVFAVGKNTVYMTSFGRGNDAVLEQKIVDTSHEPLNIDDPAYQLNVVTMNGYSVTGHRGETVSAITDNPLRRFNGGKKDKPEQSVPADMLCLKPTLEKKFFGKEFDDNIHIQLIYNILDIEKILAVYSTNAVYALNNTIADENNENWDLFANFSTDNTYGELINAATYKESTDDVSTDDEKRREAEKKKREAKIAEKILADYEKFRKNNRLAYFADAFYIEKNKSKSKSQNKAEGIKRGKKEIYSILALIAKLRHWCVHSEDGRAEFWLYKLDELEDDFKNVLDVVYNRPVEEINDDFVERNKVNIQILHSKCENSDIAELTRSYYEFLITKKYKNMGFSIKKLREIILEGTEYNDNKYDTVRNKLYQMVDFILYRGYINENSERAEALVNALRSTLNEDDKTKLYSSEAAFLKRKYMKIIREVTDSLDVKKLKELKKNAFTIPDNELRKCFISYADSVSEFTKLIYLLTRFLSGKEINDLVTTLINKFDNIRSFLEIMDELGLERTFTDEYSFFEGSTKYLAELIELNSFVKSCSFDMSAKRPMYRDALDILGIESDKSEDDIKRMIDNILQVDANGKKLPNKNHGLRNFIASNVVESNRFEYLVRYGNPKKIRETAKCKPAVRFVLNEIPDAQIERYYKAYYLDEKSLCLANMQRDKLAGVIADIKFDDFSDAGSYQKANATSTKITSEAEIKRKNQAIIRLYLTVMYIMLKNLVNVNARYVIAFHCLERDTKLYAESGLEVGNIEKNKTNLTMAVMGVKLENGIIKTEFDKSLAENAANRYLRNARWYKLILDNLKMSERAVVNEFRNTVCHLNAIRNININIDGIKEVENYFALYHYLIQKHLENRFADNGGSTGDYIGKLEEHKTYCKDFVKAYCTPFGYNLVRYKNLTIDGLFDKNYPGKDDSDKQK

SEQ ID NO: 27 - protein, 981 aa, synthetic construct; identical to SEQ ID NO: 26
  REGION 1..981: note = Cas13d_982aa

SEQ ID NO: 28 - protein, 998 aa, synthetic construct
  REGION 1..998: note = Cas13d_999aa
MGNKQRVSAQKRRENAKLCNQQKARQAESQRDKIKNMNVEKMKNINTNDIKHTKTTAKKLGLKSTIIADKKIILTSFINEQSSKTANIEKVAGFKGDTIDTISYTPRMFRSEINPGEIVISKGDDLSEFANPANFPIGRDYVKIRSALEKQYFGKEFPEDNLHVQIAYNVADIKKILSVYINNIIYMFYNLARSEEYDIFYNSQSENSGRDCDVIGSLYYQASYRNQDANRFEKDGKKKAIDSLLDDTRAYYTYFDGLFSVPKREDDGKIKESEKEKAKDQNFDVLRLLSVGRQLTFHSDKSNNEAYLFDLSKLTRAAQDENRRQDIQSLLNILNSTCRSNLEGVNGDFVKHAKNNLYVLNQLYPSLKANDLIGEYYNFIVKKENRNIGIRLITVRELIIEHNYTNLKDSKYDTYRNKIYTVLNFILFREIQENSIAIKNFREKLRSTEKAEQPALYQAFANKIYPMVQAKFAKAIDLFEEQYKTKFKSEFKGGISIENMQQQNILLQTENIDYFSKYVLFLTKFLDGKEINELLCALINKFDNIADLLDISKQIGTPVVFCADYESLNDAAKIAENIRLIKNIAHLRPAIQEAQSSKDNADAAGTPATLLIDAYNMLNTDIQLVYGEAAYEELRKDLFERKNGTKYNKKGKKVDVYDHKFRNFLINNVIKSKWFFYIAKYVKPADCAKMMSNKKMIEFALRDLPETQIKRYYYTITGNEALGDAESLKGVIIEQLHAFSIKNTLLSIKNMGEGEYKIQQIGSSKEKLKAIVNLYLTVAYLLTKSLVKVNIRFSIAFGCLERDLVLQKKSEKKFDAIINEILLEDDKIRKECDKERAQAKTLPRELAQERFAQIKRRESGCYFKSYHVYDYLSKNSNEFKQNHIDFAVTSYRNNVEHLNVVHCMTKYFSEVKDVKSYYGVYCYIMQRMLCDELIIKNQDKPDVRQTFEEYNRLLKDHGTYSKNLMWLLNFPFAYNLARYKNLSNEDLFNAKNNDQKSK

SEQ ID NO: 29 - DNA, 36 bp, synthetic construct
  misc_feature 1..36: note = Cas13bt1-2DR
  source 1..36: mol_type = other DNA
gctgaagcaaccctggttttgcggggtgattacagc

SEQ ID NO: 30 - DNA, 36 bp, synthetic construct
  misc_feature 1..36: note = Cas13bt1-5DR
  source 1..36: mol_type = other DNA
gctgtagaagcctccgatttgtgaggtgatgacagc

SEQ ID NO: 31 - DNA, 36 bp, synthetic construct
  misc_feature 1..36: note = Cas13bt1-8DR
  source 1..36: mol_type = other DNA
gctggagcagcccccgatttgtggggtaatcacagc

SEQ ID NO: 32 - DNA, 36 bp, synthetic construct
  misc_feature 1..36: note = Cas13bt1-9DR
  source 1..36: mol_type = other DNA
gttggagcagcccccgttttgtggggtaatcacaac

SEQ ID NO: 33 - DNA, 36 bp, synthetic construct
  misc_feature 1..36: note = Cas13bt1-10DR
  source 1..36: mol_type = other DNA
gctggagaagcctcggatttgcggggtgattacagc

SEQ ID NO: 34 - DNA, 36 bp, synthetic construct
  misc_feature 1..36: note = Cas13bt1-11DR
  source 1..36: mol_type = other DNA
gctggagcagccctcgatttgcagggtaatcacagc

SEQ ID NO: 35 - DNA, 36 bp, synthetic construct
  misc_feature 1..36: note = Cas13bt1-14DR
  source 1..36: mol_type = other DNA
gctggagaagcctcggttttgcgaggtgattacagc

SEQ ID NO: 36 - DNA, 36 bp, synthetic construct; identical to SEQ ID NO: 34
  misc_feature 1..36: note = Cas13bt1-15DR
  source 1..36: mol_type = other DNA

SEQ ID NO: 37 - DNA, 36 bp, synthetic construct; identical to SEQ ID NO: 34
  misc_feature 1..36: note = Cas13bt1-16DR
  source 1..36: mol_type = other DNA

SEQ ID NO: 38 - DNA, 36 bp, synthetic construct
  misc_feature 1..36: note = Cas13bt2-5DR
  source 1..36: mol_type = other DNA
gctgtgataggccccgatttgtggggtagtaacagc

SEQ ID NO: 39 - DNA, 36 bp, synthetic construct
  misc_feature 1..36: note = Cas13g1DR
  source 1..36: mol_type = other DNA
gttttcgtcacccccgttttggagggtaagtacaac

SEQ ID NO: 40 - DNA, 36 bp, synthetic construct
  misc_feature 1..36: note = Cas13g2DR
  source 1..36: mol_type = other DNA
gtccggcttaccccccatttagagggtgtccccggc

SEQ ID NO: 41 - DNA, 36 bp, synthetic construct
  misc_feature 1..36: note = Cas13g3DR
  source 1..36: mol_type = other DNA
gctggagacatccccatttctgtgggtaagccagac

SEQ ID NO: 42 - DNA, 36 bp, synthetic construct
  misc_feature 1..36: note = Cas13g4DR
  source 1..36: mol_type = other DNA
gttttcgtcaccctcgttttggagggtaagcacaac

SEQ ID NO: 43 - DNA, 36 bp, synthetic construct
  misc_feature 1..36: note = Cas13g5DR
  source 1..36: mol_type = other DNA
gtctggcttacctgccattttgcaggtgtctccagc

SEQ ID NO: 44 - DNA, 36 bp, synthetic construct
  misc_feature 1..36: note = Cas13g6DR
  source 1..36: mol_type = other DNA
gctgaagacgccctgtctatgacgggcatgtcagct

SEQ ID NO: 45 - DNA, 36 bp, synthetic construct
  misc_feature 1..36: note = Cas13g7DR
  source 1..36: mol_type = other DNA
gtcgaagacgctccgtttttgaggagcaagtacgac

SEQ ID NO: 46 - DNA, 36 bp, synthetic construct
  misc_feature 1..36: note = Cas13h1DR
  source 1..36: mol_type = other DNA
gttgtaactgcccttattttaaagggtacaaacaac

SEQ ID NO: 47 - DNA, 36 bp, synthetic construct
  misc_feature 1..36: note = Cas13i1DR
  source 1..36: mol_type = other DNA
gttgtgaaaggccgccgaaatggcggttcaagcaac

SEQ ID NO: 48 - DNA, 36 bp, synthetic construct
  misc_feature 1..36: note = Cas13j1DR
  source 1..36: mol_type = other DNA
gttgtaacaagcctaagtttgaaaggtaaaaacaac

SEQ ID NO: 49 - DNA, 36 bp, synthetic construct
  misc_feature 1..36: note = Cas13k1DR
  source 1..36: mol_type = other DNA
ggtggaaaagcctttgatttgaaaggtaaaagcacc

SEQ ID NO: 50 - DNA, 36 bp, synthetic construct
  misc_feature 1..36: note = Cas13d_896aaDR
  source 1..36: mol_type = other DNA
gagatagacccttgttaactcgtaaggttctgtgac

SEQ ID NO: 51 - DNA, 36 bp, synthetic construct
  misc_feature 1..36: note = Cas13d_911aaDR
  source 1..36: mol_type = other DNA
cttatacaacacccattttcacagtgggtctataac

SEQ ID NO: 52 - DNA, 36 bp, synthetic construct
  misc_feature 1..36: note = Cas13d_942aaDR
  source 1..36: mol_type = other DNA
gattgaaaggattgtaaatttacaaggtcttaaaac

SEQ ID NO: 53 - DNA, 36 bp, synthetic construct
  misc_feature 1..36: note = Cas13d_957aaDR
  source 1..36: mol_type = other DNA
gattgaaagctatgcgaatttgcacagtcttaaaac

SEQ ID NO: 54 - DNA, 36 bp, synthetic construct
  misc_feature 1..36: note = Cas13d_968aaDR
  source 1..36: mol_type = other DNA
gaactacagccttaacgaatgttaaggttctgaaac

SEQ ID NO: 55 - DNA, 36 bp, synthetic construct
  misc_feature 1..36: note = Cas13d_982aaDR
  source 1..36: mol_type = other DNA
gaactacacccgtgcaaaaatgcaggggtctaaaac

SEQ ID NO: 56 - DNA, 36 bp, synthetic construct
  misc_feature 1..36: note = Cas13d_999aaDR
  source 1..36: mol_type = other DNA
gtacaatagcccgttgaaatagatgggttctaagac

SEQ ID NO: 57 - RNA, 30 nt, synthetic construct
  source 1..30: mol_type = other RNA
gttcatcacgcgctcccacttgaagccctc

SEQ ID NO: 58 - RNA, 30 nt, synthetic construct
  source 1..30: mol_type = other RNA
tgcttcacgtaggccttggagccgtacatg

SEQ ID NO: 59 - RNA, 30 nt, synthetic construct
  source 1..30: mol_type = other RNA
cctctgaaacgatggtgcatggtagtgacc

SEQ ID NO: 60 - RNA, 30 nt, synthetic construct
  source 1..30: mol_type = other RNA
aattaggcagccctcatcagtgccggctcc

SEQ ID NO: 61 - RNA, 30 nt, synthetic construct
  source 1..30: mol_type = other RNA
cttgtaggctgtcctgatctcctggcgctg

SEQ ID NO: 62 - RNA, 30 nt, synthetic construct
  source 1..30: mol_type = other RNA
gagagcagcagcaaactcctttgctccctc

SEQ ID NO: 63 - RNA, 30 nt, synthetic construct
  source 1..30: mol_type = other RNA
cagaggagctcgaagtttcatctttcttct

SEQ ID NO: 64 - RNA, 30 nt, synthetic construct
  source 1..30: mol_type = other RNA
gtatcctttgattccagcacattaatggtg

SEQ ID NO: 65 - RNA, 30 nt, synthetic construct
  source 1..30: mol_type = other RNA
cttgtgaacactggggtcgtagtcaccata

SEQ ID NO: 66 - RNA, 30 nt, synthetic construct
  source 1..30: mol_type = other RNA
tagtcaccatacttggcctggacggcgtaa

SEQ ID NO: 67 - RNA, 30 nt, synthetic construct
  source 1..30: mol_type = other RNA
cctttttggaagcaattcctcttgggccaa

SEQ ID NO: 68 - RNA, 30 nt, synthetic construct
  source 1..30: mol_type = other RNA
ctgtactggtggatgtcctcaaaagacttg

SEQ ID NO: 69 - RNA, 30 nt, synthetic construct
  source 1..30: mol_type = other RNA
tccgagtccttcacccgtttgatctgctcc

SEQ ID NO: 70 - RNA, 30 nt, synthetic construct
  source 1..30: mol_type = other RNA
tgttccccaccagcaccatgggcacgtcat

SEQ ID NO: 71 - RNA, 30 nt, synthetic construct
  source 1..30: mol_type = other RNA
taccagtgtgtaaaaagcatcttcaacacc

SEQ ID NO: 72 - RNA, 30 nt, synthetic construct
  source 1..30: mol_type = other RNA
ctgtagaggttaatatccgcaaatgacttg

SEQ ID NO: 73 - RNA, 30 nt, synthetic construct
  source 1..30: mol_type = other RNA
cgcttaatctgctccctgtagaggttaata

SEQ ID NO: 74 - RNA, 30 nt, synthetic construct
  source 1..30: mol_type = other RNA
cattatgagacatccccactgcaaggcatt

SEQ ID NO: 75 - RNA, 30 nt, synthetic construct
  source 1..30: mol_type = other RNA
cggcctgtggcatccgcccaaacctgatgg

SEQ ID NO: 76 - RNA, 30 nt, synthetic construct
  source 1..30: mol_type = other RNA
aaacctgatggcattatgagacatccccac

SEQ ID NO: 77 - RNA, 30 nt, synthetic construct
  source 1..30: mol_type = other RNA
tagcatccatgatcttatagcccatgatga

SEQ ID NO: 78 - RNA, 30 nt, synthetic construct
  source 1..30: mol_type = other RNA
gtttctggcagttctcctctcctgcacccc

SEQ ID NO: 79 - RNA, 30 nt, synthetic construct
  source 1..30: mol_type = other RNA
gaaagccctccccagtcctcatgtactggt

SEQ ID NO: 80 - RNA, 30 nt, synthetic construct
  source 1..30: mol_type = other RNA
ataaggccaaccatgatgtgctgaaactgg

SEQ ID NO: 81 - RNA, 27 nt, synthetic construct
  source 1..27: mol_type = other RNA
gccggcactgatgagggctgcctaatt

(19) United States
(12) Patent Application Publication
(10) Pub. No.: US 2025/0290055 A1
(43) Pub. Date: Sep. 18, 2025

(54) ISOLATED CAS13 PROTEINS, GENE EDITING SYSTEM BASED THEREON, AND USE THEREOF

(71) Applicants: BEIJING INSTITUTE FOR STEM CELL AND REGENERATIVE MEDICINE, Beijing (CN); INSTITUTE OF ZOOLOGY, CHINESE ACADEMY OF SCIENCES, Beijing (CN)

(72) Inventors: Wei LI, Beijing (CN); Qi ZHOU, Beijing (CN); Yanping HU, Beijing (CN); Yangcan CHEN, Beijing (CN)

(73) Assignees: BEIJING INSTITUTE FOR STEM CELL AND REGENERATIVE MEDICINE, Beijing (CN); INSTITUTE OF ZOOLOGY, CHINESE ACADEMY OF SCIENCES, Beijing (CN)

(21) Appl. No.: 18/860,225

(22) PCT Filed: Jun. 20, 2023
(86) PCT No.: PCT/CN2023/101348

§ 371 Date: Oct. 25, 2024

(30) Foreign Application Priority Data
Apr. 26, 2022 (CN) 202210446714.X
Apr. 24, 2023 (CN) 202310447170.3

(51) Int. Cl. C12N 9/22; C12N 9/78; C12N 15/11

(52) U.S. Cl. CPC C12N 9/226; C12N 9/78; C12N 15/111; C07K 2319/80; C12N 2310/20; C12Y 305/04004; C12Y 305/04005

(57) ABSTRACT

The present application relates to isolated novel CRISPR/Cas13 proteins, a gene editing systems based thereon, and a method for using said proteins for RNA level gene editing. Provided are non-naturally occurring or engineered RNA targeting systems, and said systems each have a novel Cas13 effector protein that targets RNA and at least one type of guide molecule.

### TECHNICAL FIELD

The present application is in the field of biotechnology and specifically relates to an isolated Cas13 protein, a gene editing system based on it and a method of using them for RNA level gene editing.

### TECHNICAL BACKGROUND

Gene editing technology makes it possible to modify DNA sequence anchor points, such as the first-generation gene editing tools, zinc finger nucleases (ZFNs), and the second-generation gene editing tools, transcription activator-like effector nucleases (TALENs), the third generation gene editing tools, type II and V clustered regularly interspaced short palindromic repeats (CRISPR)/Cas (CRISPR-associated proteins) can be used for targeted genome modification, but these gene editing systems can only target the genome and foreign DNA, but cannot perform targeted editing on RNA in vivo.

Type VI CRISPR/Cas13 system is the natural immune system from archaea and bacteria, which is different from previous gene editing tools, it uses the principle of complementary nucleic acid base pairing to identify target RNA sequences and guide Cas effector proteins for targeted cleavage. It has strong applicability, simple design, and high efficiency.

Among them, type VI-D CRISPR/Cas13 gene editing system is the most widely used type VI CRISPR/Cas system. This system can perform targeted editing of single-stranded RNA in prokaryotes by recognizing and cutting the protospacer flanking site (PFS) sequence on both sides of the targeted polynucleotide. And in eukaryotes, the VI-D type CRISPR/Cas system has no PFS sequence for targeted editing of RNA. At the same time, compared with other type VI systems, the type VI-D system has a smaller protein size and has better prospects in gene therapy based on adeno-associated virus (AAV). In the huge and diverse metagenomes, there are hidden microorganisms that have not been cultured or even discovered. There may be a large number of undiscovered type VI CRISPR/Cas13 systems. Finding and cloning Cas proteins with better characteristics among these microorganisms is what people expect.

### SUMMARY OF THE INVENTION

The purpose of this application is to find novel Cas13 gene editing systems in metagenomes and develop their uses. Specifically, provided is that:

1. Anisolated Cas13 nuclease protein, the amino acid sequence of the Cas13 protein comprises:
a) the amino acid sequence shown in any one of SEQ ID NOs. 1 to 28; or
b) an amino acid sequence having at least 80% sequence identity with any one of SEQ ID NOs. 1 to 28 and having RNA cleavage activity.

2. The Cas13 nuclease protein of item 1, wherein the Cas13 nuclease protein is Cas13bt1, Cas13bt2, Cas13g, Cas13h, Cas13i, Cas13j or Cas13k protein.

3. An engineered Cas13 nuclease effector protein, comprising:
a) the amino acid sequence shown in any one of SEQ ID NOs. 1 to 28; or,
b) an amino acid sequence having at least 80% sequence identity with any one of SEQ ID NOs. 1 to 28 and having RNA cleavage activity.

4. The effector protein of item 3, wherein the Cas13 nuclease protein loses its catalytic activity through amino acid mutation, for example, the HEPN domain (RxxxxH motif) of the Cas13 nuclease protein at its C-terminal and/or N-terminal forms dCas13 protein through amino acid mutations.

5. The effector protein of item 3 or 4, further comprising a functional domain fused to the Cas13 nuclease protein.

6. The effector protein of item 5, wherein the functional domain is selected from one or more of the following: translation initiation domain, translation repression domain, transactivation domain, epigenetic modification domain, nucleobase editing domain, reverse transcriptase domain, reporter domain and nuclease domain.

7. The effector protein of item 6, wherein the nucleobase editing domain is adenosine deaminase, cytidine deaminase or their catalytic domains.

8. A polynucleotide, encoding the Cas13 nuclease protein of item 1 or 2 or the effector protein of any one of items 3-7.

9. A vector, comprising the polynucleotide of item 8, preferably the vector is a plasmid or a lentivirus.

10. An engineered CRISPR-Cas13 gene editing system, comprising:
(a) the engineered Cas13 nuclease effector protein of any one of items 3-7, or the nucleic acid encoding the effector protein; and
(b) crRNA, comprising a spacer sequence complementary to a target sequence, when using engineered Cas13 nuclease effector proteins as shown in SEQ ID NOs. 1-28, the crRNA also comprises direct repeat (DR) sequences that have at least 80% identity with the sequences shown in SEQ ID NOs. 29-56;
   wherein, the engineered Cas13 nuclease effector protein and the crRNA can form a CRISPR complex that specifically binds to a target nucleic acid comprising the target sequence and induces modification of the target nucleic acid.

11. A kit, comprising the engineered CRISPR-Cas13 gene editing system of item 10.

12. Use of the engineered CRISPR-Cas13 nuclease effector protein of any one of items 3-7, the engineered CRISPR-Cas13 gene editing system of item 10 in the preparation of a medicament for the treatment of diseases or disorders associated with nucleic acid mutations in cells of an individual.

13. A method for modifying a cell comprising a target nucleic acid, comprising contacting the cell with the engineered Cas13 nuclease effector protein of any one of items 3-7, the engineered CRISPR-Cas13 gene editing system of item 10, thereby achieving modification of the target nucleic acid in the cell.

14. Use of the engineered Cas13 nuclease effector protein of any one of items 3-7, the engineered CRISPR-Cas13 gene editing system of item 10 in the preparation of a medicament for the treatment of diseases or disorders associated with nucleic acid mutations in an individual.

This application also provides:

1. An isolated Cas13 nuclease protein, the amino acid sequence of the Cas13 nuclease protein is:
a) the amino acid sequence shown in any one of SEQ ID NOs. 1 to 28; or
b) an amino acid sequence having at least 80% sequence identity with any one of SEQ ID NOs. 1 to 28 and having RNA cleavage activity.

2. The Cas13 nuclease protein of item 1, wherein the Cas13 nuclease protein is Cas13bt1, Cas13bt2, Cas13g, Cas13h, Cas13i, Cas13j or Cas13k protein, preferably Cas13g3.

3. An engineered Cas13 nuclease effector protein, comprising a Cas13 nuclease protein, the Cas13 nuclease protein comprising:
a) the amino acid sequence shown in any one of SEQ ID NOs. 1 to 28; or,
b) an amino acid sequence having at least 80% sequence identity with any one of SEQ ID NOs. 1 to 28 and having RNA cleavage activity.

4. The effector protein of item 3, wherein the Cas13 nuclease protein loses its catalytic activity through amino acid mutation, for example, the HEPN domain (RxxxxH motif) of the Cas13 nuclease protein at its C-terminal and/or N-terminal forms dCas13 protein through amino acid mutations.

5. The effector protein of item 3 or 4, further comprising a functional domain fused to the Cas13 nuclease protein.

6. The effector protein of item 5, wherein the functional domain is selected from one or more of the following: translation initiation domain, translation repression domain, transactivation domain, epigenetic modification domain, nucleobase editing domain, reverse transcriptase domain, reporter domain and nuclease domain.

7. The effector protein of item 6, wherein the nucleobase editing domain is adenosine deaminase, cytidine deaminase or their catalytic domains.

8. A polynucleotide, encoding the Cas13 nuclease protein of item 1 or 2 or the effector protein of any one of items 3-7.

9. A vector, comprising the polynucleotide of item 8, wherein the vector is a plasmid or a virus, and the virus is preferably a lentivirus.

10. An engineered CRISPR-Cas13 gene editing system, comprising:
(a) the nuclease of item 1 or 2, the engineered Cas13 nuclease effector protein of any one of items 3-7, or the nucleic acid encoding the effector protein; and
(b) crRNA, comprising a spacer sequence complementary to a target sequence in a target nucleic acid;
   wherein, the engineered Cas13 nuclease effector protein and the crRNA can form a CRISPR complex that specifically binds to a target nucleic acid comprising the target sequence and induces modification of the target nucleic acid.

11. The engineered CRISPR-Cas13 gene editing system of item 10, wherein the crRNA further comprises a direct repeat (DR) sequence, preferably the direct repeat (DR) sequence comprises any one of SEQ ID NOs. 29 to 56. or a sequence having at least 80% identity with the sequence shown in any one of SEQ ID NOs. 29 to 56.

12. A kit, comprising the engineered CRISPR-Cas13 gene editing system of item 10 or 11.

13. Use of the Cas13 nuclease protein of item 1 or 2, the engineered CRISPR-Cas13 nuclease effector protein of any one of items 3-7, the engineered CRISPR-Cas13 gene editing system of item 10 or 11 in the preparation of a medicament for the treatment of diseases or conditions associated with nucleic acid mutations in cells of an individual.

14. A method for modifying a cell comprising a target nucleic acid, comprising contacting the cell with the Cas13 nuclease protein of item 1 or 2, the engineered Cas13 nuclease effector protein of any one of items 3-7, the engineered CRISPR-Cas13 gene editing system of item 10 or 11, thereby achieving modification of the target nucleic acid in the cell.

15. A composition comprising the Cas13 nuclease protein of item 1 or 2, the engineered Cas13 nuclease effector protein of any one of items 3-7, the engineered CRISPR-Cas13 gene editing system of item 10 or 11, for modification of nucleic acids.

### Beneficial Technical Effects Achieved by the Technical Solution of This Application

This application analyzed metagenomic data and finally identified 5 novel isoforms and a total of 75 Cas13 proteins. The novel Cas13 isoforms are named as Cas13g-k; wherein the Cas13g protein is approximately 800 amino acids in size, and the Cas13h-k protein is approximately 1100 amino acids in size. These novel proteins all demonstrate good targeted cleavage activity at the RNA level. We report that the newly discovered Cas13g3 protein can be engineered for programmable RNA editing.

Cas13g3 has a size of 767aa and is considered to be the most efficient small Cas13 protein currently and can be used as a promising mammalian RNA editing tool.

### BRIEF DESCRIPTION OF THE DRAWINGS

FIG. 1 shows the flow chart of identification of a total of 75 Cas13 proteins in 5 novel isoforms through analysis of metagenomic data.

FIG. 2 shows a schematic diagram of the protein homology between the 7 isoforms and the 4 old isoforms and the average protein size of each isoform.

FIG. 3 shows another schematic diagram of the protein homology between the 7 isoforms and the 4 old isoforms.

FIG. 4 shows a schematic diagram of the spacer-directed repeat sequence of the Loci site of Cas13bt1-Cas13k.

FIG. 5 shows a schematic diagram of the HEPN domain at the C-terminus and N-terminus of Cas13bt1-Cas13k.

FIG. 6 shows the ratio of RxxxxH motifs of Cas13bt1-Cas13k.

FIG. 7 is a schematic diagram showing the sequence conservation of the RxxxxH motif of the HEPN domain at the C-terminus and N-terminus of Cas13bt1-Cas13k.

FIG. 8 shows a schematic diagram of the predicted structure of the DR sequence of Cas13bt1-Cas13k.

FIG. 9 shows the distribution ratio of Cas13a-Cas13k in microorganisms in various environments.

FIG. 10 shows the existing ratio of Cas13a-Cas13k in nature.

FIGS. 11A and 11B are schematic diagrams of the experimental process for analyzing the RNA cleavage activity of Cas13 protein in HEK293T cells.

FIG. 12 is a comparison of the efficiency of knocking down mCherry mRNA using 3′-DR or 5′-DR-crRNA.

FIGS. 13A and 13B show the results of knocking down mCherry mRNA to screen Cas13 proteins with high editing efficiency.

FIGS. 14A and 14B are real time quantitative reverse transcription PCR (RT-qPCR) results of novel Cas13-mediated ANXA4 mRNA knockdown.

FIG. 15 shows the quantitative RT-qPCR results of endogenous mRNA knockdown mediated by RfxCas13d, Cas13X1, Cas13bt1-11 and Cas13g3.

FIG. 16 shows the RT-qPCR results of novel Cas13d-mediated knockdown of ANXA4 mRNA.

FIG. 17 shows the RNA-seq analysis results of ANXA4 mRNA knockdown mediated by RfxCas13d, Cas13X1, Cas13bt1-11 and Cas13g3.

FIGS. 18A and 18B show the trans-activity assessment of RfxCas13d, Cas13X1, Cas13bt1-11 and Cas13g3.

FIGS. 19A and 19B show analysis of optimal spacer length for the Cas13g3 system.

FIG. 20 shows Cas13g3 system PFS preference analysis.

FIG. 21 shows the effect of NLS interference activity on the Cas13g3 system.

FIG. 22 shows a comparison of the RNA interference capabilities of the Cas13g3 system and other Cas13 systems.

### DETAIL DESCRIPTION OF THE PRESENT INVENTION

Specific embodiments of the present invention will be described in more detail below with reference to the accompanying drawings. Although specific embodiments of the present invention are shown in the drawings, it should be understood that the present invention may be embodied in various forms and should not be limited to the embodiments set forth herein. Rather, these embodiments are provided to provide a thorough understanding of the present invention, and to fully convey the scope of the present invention to those skilled in the art.

It should be noted that certain words are used in the description and claims to refer to specific components. Those skilled in the art will understand that skilled persons may use different names to refer to the same component. This specification and the claims do not use difference in nouns as a way to distinguish components, but rather use differences in functions of the components as a criterion for distinction. If the words “comprise” or “include” mentioned throughout the specification and claims are open-ended terms, they should be interpreted as “include but not limited to.” The following descriptions of the specification are preferred embodiments for implementing the present invention. However, the descriptions are for the purpose of general principles of the specification and are not intended to limit the scope of the present invention. The protection scope of the present invention shall be determined by the appended claims.

As used herein, “substantially free” with respect to a particular component is used herein to mean that the particular component is not purposefully formulated into the composition and/or is present only as a contaminant or in trace amounts. Therefore, the total amount of a particular component resulting from any accidental contamination of the composition is less than 0.05%, preferably less than 0.01%. Most preferred are compositions in which the specific component is present in an amount undetectable by standard analytical methods.

As used in this specification, “a” or “an” may mean one or more. As used in the claims, the word “a” or “an” when used with the word “comprising” can mean one or more than one.

The term “or” is used in the claims to mean “and/or” unless it is expressly stated that only alternatives are to be referred to or the alternatives are mutually exclusive, although this disclosure supports reference to only alternatives and “and/or” definition. As used herein, “another” may mean at least a second or more.

Throughout this application, the term “about” is used to indicate that a value includes the inherent variation in error of the device, the method used to determine the value, or the variation that exists between study subjects.

In a first aspect, the present application provides an isolated Cas13 nuclease protein.

In a specific embodiment, provided is an isolated Cas13 nuclease protein, and the amino acid sequence of the Cas13 protein comprises: a) the amino acid sequence shown in any one of SEQ ID NO. 1 to 28; or, b) an amino acid sequence having at least 80% sequence identity with any one of SEQ ID NOs. 1 to 28 and having RNA cleavage activity. In yet another specific embodiment, provided is a Cas13 nuclease protein, wherein the Cas13 protein is Cas13bt1, Cas13bt2, Cas13g, Cas13h, Cas13i, Cas13j or Cas13k protein.

In the context of this specification, the terms Cas13a and C2c2 are used interchangeably. It was originally discovered by researchers at the Broad Institute. In 2015, Shmakov and colleagues used Cas1 as a “bait” to identify CRISPR-associated proteins in bacterial genomes. Through this analysis, they identified 53 candidate genes, divided into three major categories: C2c1, C2c2 and C2c3. C2c1 and C2c3 are somewhat similar to Cpf1, except that they require both tracrRNA and crRNA to cleave DNA targets, whereas Cpf1 only requires crRNA. The difference between C2c2 (that is, Cas13a) and Cas9 is that Cas13a binds and cuts RNA, while Cas9 cuts DNA. Cas9 utilizes tracrRNA and crRNA to bind and cleave DNA targets. Cas13a only requires a 24-base crRNA, which interacts with the Cas13a molecule through a uracil-rich stem-loop structure and promotes target cleavage through a series of conformational changes of Cas13a. Like Cas9, Cas13a can also tolerate a single mismatch between crRNA and the target sequence, but if there are two mismatches, the cutting efficiency is greatly reduced. Its PFS sequence (equivalent to PAM sequence) is located at the 3′ end of the spacer region and consists of A, U or C bases. Another special feature of Cas13a is that once Cas13a recognizes and cleaves the RNA target specified by the crRNA sequence, it enters an enzymatic “activation” state, at which time it will bind and cleave other RNAs regardless of whether they are homologous to crRNA, or whether there is a PFS; this is very different from Cas9. In the Cas13 system, tracrRNA is not required, only crRNA is required; and crRNA is composed of a DR sequence (that is, a direct repeat sequence) and a spacer sequence (a spacer sequence, that is, a nucleotide sequence complementary to the target sequence).

In the second aspect, the present application provides an engineered Cas13 nuclease effector protein In a specific embodiment, provided is an engineered Cas13 nuclease effector protein, comprising: a) an amino acid sequence as shown in any one of SEQ ID NO. 1 to 28; or, b) an amino acid sequence having at least 80% sequence identity with any one of SEQ ID NOs. 1 to 28 and having RNA cleavage activity. Preferably, the nuclease effector protein has 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% or more of sequence identity with any one of SEQ ID NOs. 1 to 28.

In a specific embodiment, provided is an engineered Cas13 nuclease effector protein. The Cas13 nuclease effector protein loses its catalytic activity through amino acid mutation. For example, the Cas13 nuclease protein undergoes mutations in a HEPN structure at its C-terminus and N-terminus (RxxxxH motif) to form the dCas13 protein.

In the context of this specification, the term HEPN refers to the two domains contained in Cas13a, which act on Cas13 equivalent to the HNH and RuvC domains on Cas9, to cleave target nucleic acids. The HEPN domain is required for Cas13a to cleave RNA targets. The RxxxxH motifs of the HEPN ribonuclease domain are located at the N-terminus and C-terminus of the Cas13 protein respectively. In addition, similar to Cas9, mutations of key residues in the Cas13a molecule can form Cas13a with missing nuclease activity (dCas13a), which can bind to RNA targets but cannot cleave. The RxxxxH motif is 6 consecutive amino acids, the first amino acid is arginine R, the last one is histidine H, and there can be any amino acid in the middle. The RxxxxH motif is the HEPN motif and has ribonuclease function.

In a specific embodiment, provided is an engineered Cas13 nuclease effector protein, further comprising a functional domain fused to the Cas13 nuclease protein. Wherein, the functional domain is selected from one or more of the following: translation initiation domain, translation repression domain, transactivation domain, epigenetic modification domain, nucleobase editing domain, reverse transcriptase domain, reporter domain and nuclease domain; wherein the nucleobase editing domain is adenosine deaminase, cytidine deaminase or their catalytic domain fusion.

In the third aspect the application relates to a polynucleotide.

In a specific embodiment, provided is a polynucleotide encoding the aforementioned Cas13 nuclease protein or the aforementioned engineered effector protein.

In the fourth aspect, the present application relates to a polynucleotide vector.

In a specific embodiment, provided is a polynucleotide vector, preferably the vector is a plasmid or lentivirus.

In the fifth aspect, the present application relates to a gene editing system.

In a specific embodiment, provided is an engineered CRISPR-Cas13 gene editing system, comprising: (a) the aforementioned engineered Cas13 nuclease effector protein or nucleic acid encoding the effector protein; and (b) crRNA, comprising a spacer sequence complementary to the target sequence. When using an engineered Cas13 nuclease effector protein as shown in SEQ ID NOs. 1 to 28, the crRNA also comprises a direct repeat (DR) sequence with at least 80% sequence identity with the sequences as shown in SEQ ID NOs. 29 to 56, respectively; wherein the engineered Cas13 nuclease effector protein and the crRNA are capable of forming a CRISPR complex that specifically binds to the target sequence comprising the target sequence and induce modification of the target nucleic acid. Preferably, the direct repeat (DR) sequence has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the sequence as shown in SEQ ID NOs. 29 to 56.

In a sixth aspect, the present application relates to a kit.

In a specific embodiment, provided is a kit comprising the aforementioned engineered CRISPR-Cas13 gene editing system.

In the seventh aspect, the application relates to the use of engineered CRISPR-Cas13 nuclease effector proteins.

In a specific embodiment, provided is use of the aforementioned engineered CRISPR-Cas13 nuclease effector protein and the aforementioned engineered CRISPR-Cas13 gene editing system in preparing a medicament for treating diseases or disorders related to nucleic acid mutations in the cells of an individual.

In the eighth aspect, the application relates to a method of modifying a target nucleic acid contained in a cell.

In a specific embodiment, provided is a method for modifying a cell containing a target nucleic acid, comprising contacting the cell with the aforementioned engineered CRISPR-Cas13 nuclease protein effector protein and the aforementioned engineered CRISPR-Cas13 gene editing system, thereby achieving modification of the target nucleic acid in the cell.

In the ninth aspect, the present application relates to a use for treating a disease or disorder associated with nucleic acid mutations in an individual. In a specific embodiment, provided is the aforementioned engineered CRISPR-Cas13 nuclease effector protein or the aforementioned engineered CRISPR-Cas13 gene editing system in preparing a medicament for treating diseases or disorders related to nucleic acid mutations in an individual.

### EXAMPLE

### Example 1: Identification of Novel Cas13 Isoforms

### Searching for Novel Cas13

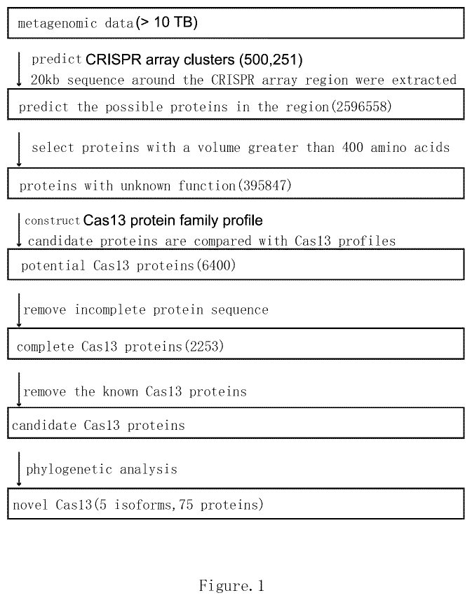

Nearly 10 Tb (Terabyte) assembled metagenomic data, mainly from human and animal hosts, aquatic, natural and agricultural soils were downloaded from the JGI (Joint Genome Institute) database. approximately 500,251 CRISPR arrays were predicted by MinCED software, 20 kb (kilobase) sequences upstream and downstream of the CRISPR array region were extracted as candidate sequences, and protein open reading frame (ORF) on the candidate sequences were performed by Meta-GeneMark 1, and 2,596,558 candidate proteins were obtained. Given the known size of Cas proteins, we selected proteins with a protein size greater than 400 amino acids as candidate proteins and obtained 395,847 proteins. The known Cas13a, Cas13b, Cas13c and Cas13d sequences from NCBI (National Center for Biotechnology Information) were downloaded, and a known Cas13 protein database was constructed by HH-suite3 software. 395847 candidate proteins were compared with the known Cas13 protein database through hhsearch of HH-suite3 software, and 6400 proteins with comparison results were obtained. After removing incomplete proteins and proteins containing less than 2 higher eukaryotes and prokaryotes nuceotide-binding (HEPN) domain proteins from candidate proteins, 2253 candidates were obtained. After removing the known Cas13 protein sequences, 1139 novel Cas13s were obtained (FIG. 1).

### Identification of the Novel Cas13 Isoforms by Phylogenetic Analysis

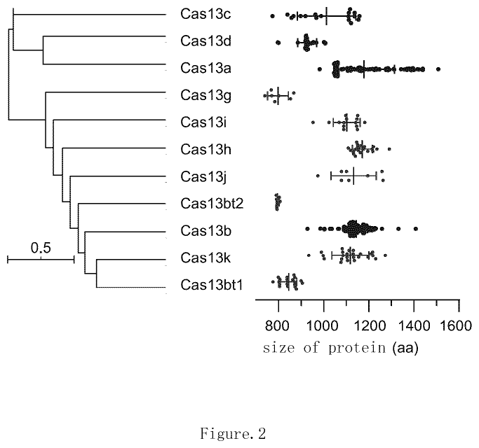

Phylogenetic analysis and classification of candidate proteins were performed. The 2253 candidate proteins were firstly clustered through preliminary clustering using the cluster—min-seq-id 0.5-c 0.7 parameter of the MMseqs software. Then redundant proteins were removed from each cluster through the cluster—min-seq-id 0.9-c 0.8 parameter of the mmseqs software. Each cluster was then subjected to multiple sequence alignment through MAFFT software. The alignment results were used to construct a hidden Markov model using hhmake of HH-suite3 software. Then the hidden Markov model files were compared with each other using hh-align of the HH-suite3 software. Alignment scores between proteins were used to calculate evolutionary distance, wherein sij represents the alignment score between i protein and j protein; sji represents the alignment score between j protein and i protein; Sij represents the average alignment score between i protein and j protein; dij represents the evolutionary distance between i protein and j protein. The calculation method of evolutionary distance was Sij=(sij+sji)/2, dij=(log(min(Sii,Sjj))−log (Sij))/2. Evolutionary distance results were performed cluster analysis using the unweighted group average method (UPGMA). Finally, many known Cas13a-d homologs and 7 different branches were identified. Wherein two of the branches were homologous to the previously identified Cas13X, Cas13Y, and Cas13bt, and we named them as Cas13bt1 and Cas13bt2. The remaining five branches are novel isoforms, which we named as Cas13g-k. Wherein the size of Cas13g protein was about 800 amino acids, and the size of Cas13h-k protein was about 1100 amino acids (FIGS. 1 and 2).

### Analysis of Cas13 Isoform Evolutionary Tree

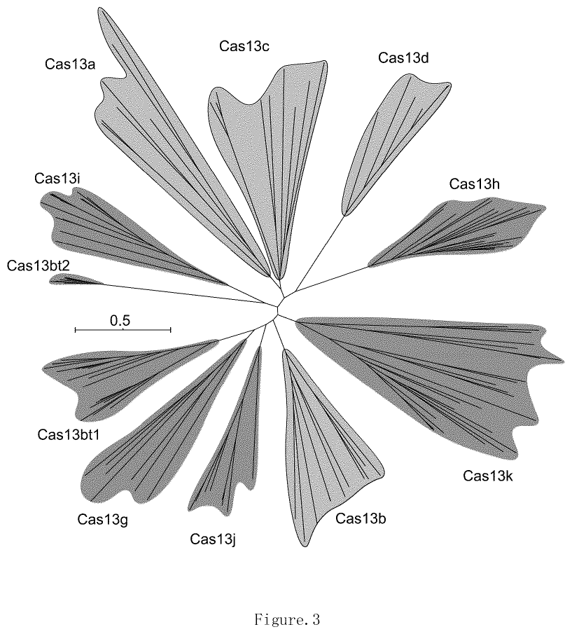

The novel Cas13 isoform protein and some known Cas13 proteins were subjected to multiple sequence alignment through MUSCLE software. The alignment results were used to construct an evolutionary tree through FastTree software, and finally the evolutionary tree was presented through the ITOL website (FIG. 3).

### Example 2: Characterization of Novel Cas13 Isoforms

### CRISPR Loci Analysis of Novel Cas13 Isoforms

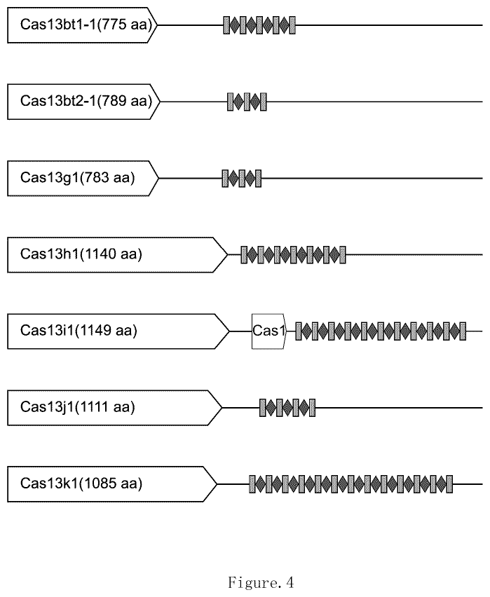

The 20 kb sequence upstream and downstream of the CRISPR array region was extracted for ORF prediction, and the obtained protein sequences were compared using blast software. Cas13i had the CRISPR conserved proteins Cas1 and Cas2, while the CRISPR Loci corresponding to other Cas13 isoforms lacked these conserved proteins (FIG. 4).

### Analysis of HEPN Domain of Cas13 Isoforms

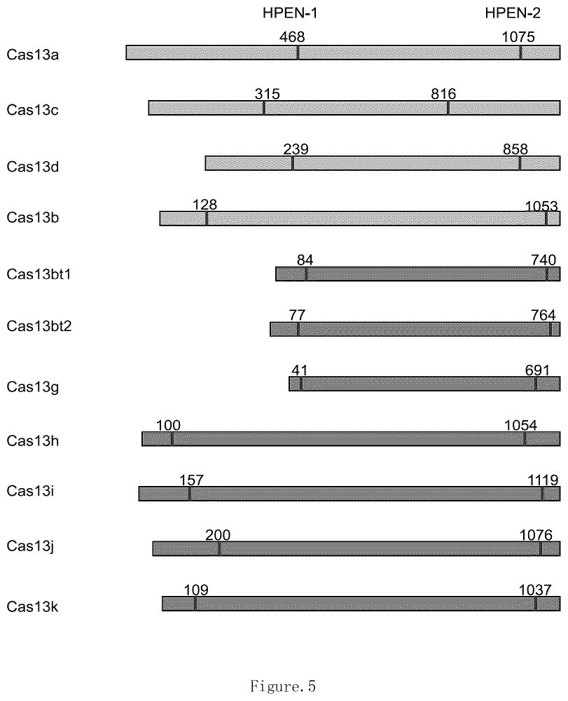

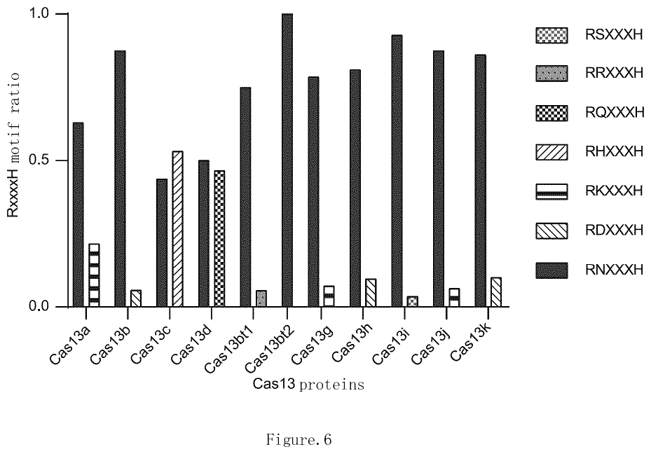

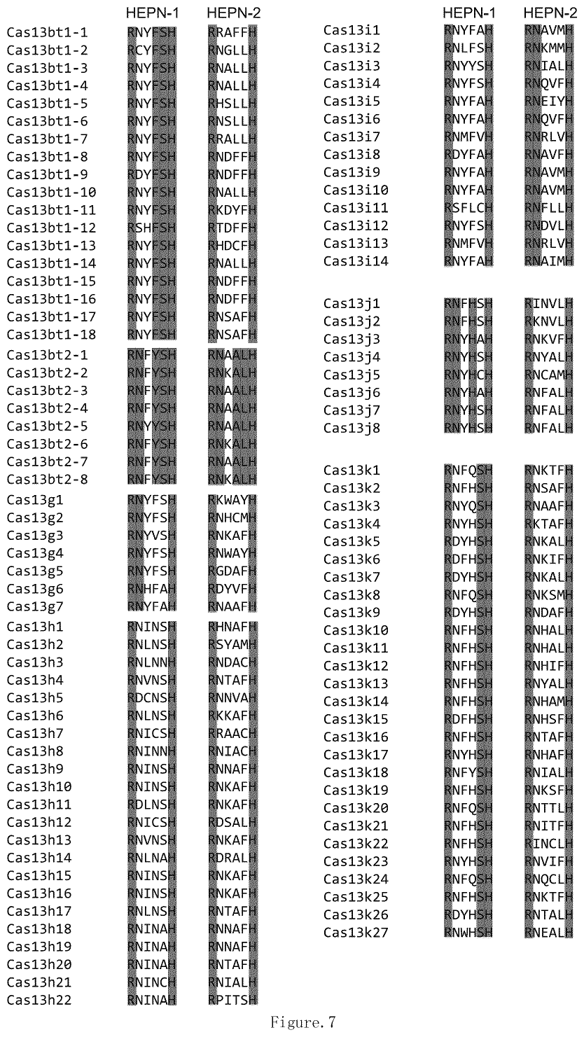

Multiple sequence alignment was performed on each isoform protein sequence using MUSCLE software, and the HEPN domain information of each protein was extracted from the alignment results. The distribution map of representative proteins corresponding to the HEPN domain in each Cas13 isoform was drawn (FIG. 5). The RXXXXH motifs corresponding to each Cas13 isoform protein were sorted to show the proportion of the first two motifs (FIG. 6). Finally, the conservation of the HEPN domain of the novel Cas13 isoform protein was analyzed (FIG. 7).

### CRISPR Array Analysis of Novel Cas13 Isoforms

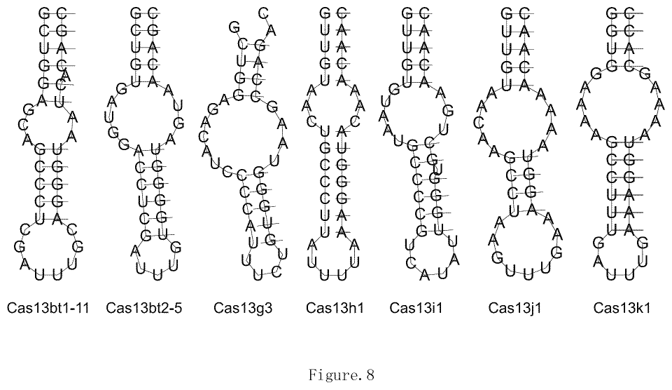

The direct repeat sequences (DR) were found by using MinCED software. The specific DR sequence information was shown in Table 1. The secondary structure of the DR sequence of the novel Cas13 isoform was predicted through the RNAfold website, showing that the predicted structure was also similar to the previous structures of Cas13a and Cas13b (FIG. 8). The spacer corresponding to the novel Cas13 isoform was predicted through CRISPR target, and some of the sequences were compared to the IMG/VR database, indicating that the novel Cas13 isoform proteins may help microorganisms defend against foreign mobile genetic factors.

TABLE 1

| SEQ | | |
|---|---|---|
| ID | CAS | |
| NO. | enzyme | DR sequence |
| 29 | Cas13bt1- | GCTGAAGCAACCCTGGTTTTGCGGGGTGATTACAGC |
| | 2 | |
| 30 | Cas13bt1- | GCTGTAGAAGCCTCCGATTTGTGAGGTGATGACAGC |
| | 5 | |
| 31 | Cas13bt1- | GCTGGAGCAGCCCCCGATTTGTGGGGTAATCACAGC |
| | 8 | |
| 32 | Cas13bt1- | GTTGGAGCAGCCCCCGTTTTGTGGGGTAATCACAAC |
| | 9 | |
| 33 | Cas13bt1- | GCTGGAGAAGCCTCGGATTTGCGGGGTGATTACAGC |
| | 10 | |
| 34 | Cas13bt1- | GCTGGAGCAGCCCTCGATTTGCAGGGTAATCACAGC |
| | 11 | |
| 35 | Cas13bt1- | GCTGGAGAAGCCTCGGTTTTGCGAGGTGATTACAGC |
| | 14 | |
| 36 | Cas13bt1- | GCTGGAGCAGCCCTCGATTTGCAGGGTAATCACAGC |
| | 15 | |
| 37 | Cas13bt1- | GCTGGAGCAGCCCTCGATTTGCAGGGTAATCACAGC |
| | 16 | |
| 38 | Cas13bt2- | GCTGTGATAGGCCCCGATTTGTGGGGTAGTAACAGC |
| | 5 | |
| 39 | Cas13g1 | GTTTTCGTCACCCCCGTTTTGGAGGGTAAGTACAAC |
| 40 | Cas13g2 | GTCCGGCTTACCCCCCATTTAGAGGGTGTCCCCGGC |
| 41 | Cas13g3 | GCTGGAGACATCCCCATTTCTGTGGGTAAGCCAGAC |
| 42 | Cas13g4 | GTTTTCGTCACCCTCGTTTTGGAGGGTAAGCACAAC |
| 43 | Cas13g5 | GTCTGGCTTACCTGCCATTTTGCAGGTGTCTCCAGC |
| 44 | Cas13g6 | GCTGAAGACGCCCTGTCTATGACGGGCATGTCAGCT |
| 45 | Cas13g7 | GTCGAAGACGCTCCGTTTTTGAGGAGCAAGTACGAC |
| 46 | Cas13h1 | GTTGTAACTGCCCTTATTTTAAAGGGTACAAACAAC |
| 47 | Cas13il | GTTGTGAAAGGCCGCCGAAATGGCGGTTCAAGCAAC |
| 48 | Cas13j1 | GTTGTAACAAGCCTAAGTTTGAAAGGTAAAAACAAC |
| 49 | Cas13kl | GGTGGAAAAGCCTTTGATTTGAAAGGTAAAAGCACC |
| 50 | Cas13d_ | GAGATAGACCCTTGTTAACTCGTAAGGTTCTGTGAC |
| | 896 aa | |
| 51 | Cas13d_ | CTTATACAACACCCATTTTCACAGTGGGTCTATAAC |
| | 911 aa | |
| 52 | Cas13d_ | GATTGAAAGGATTGTAAATTTACAAGGTCTTAAAAC |
| | 942 aa | |
| 53 | Cas13d_ | GATTGAAAGCTATGCGAATTTGCACAGTCTTAAAAC |
| | 957 aa | |
| 54 | Cas13d_ | GAACTACAGCCTTAACGAATGTTAAGGTTCTGAAAC |
| | 968 aa | |
| 55 | Cas13d_ | GAACTACACCCGTGCAAAAATGCAGGGGTCTAAAAC |
| | 982 aa | |
| 56 | Cas13d_ | GTACAATAGCCCGTTGAAATAGATGGGTTCTAAGAC |
| | 999 aa | |

### Novel Cas13 Distribution

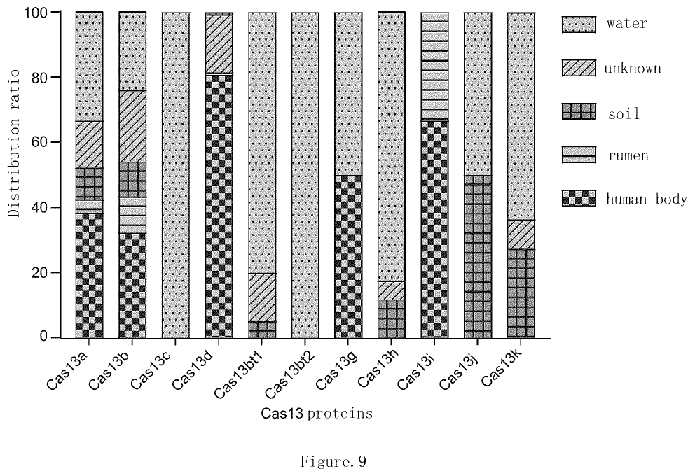

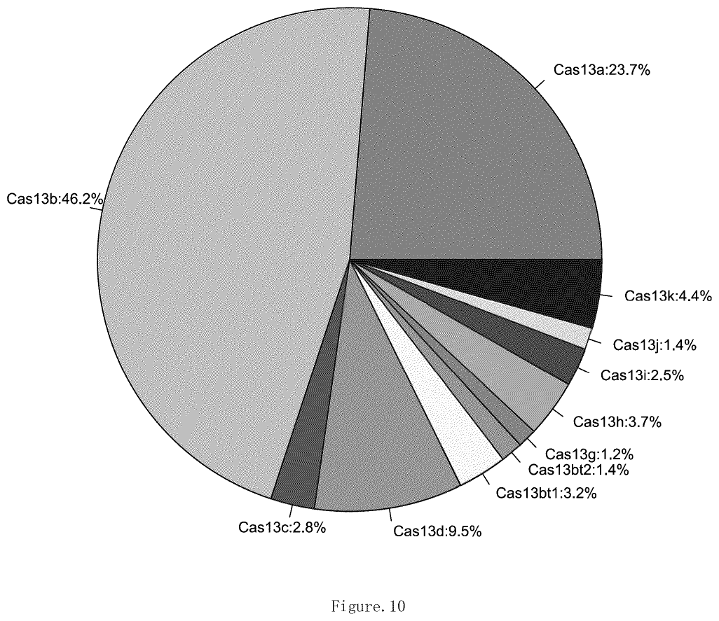

Statistics on the classification and metagenomic sources of all novel Cas13s (1139) found that 80% of Cas13d originated from human microorganisms, Cas13c came from water sources, more than 30% of Cas13a came from human microorganisms or water sources, and Cas13b was distributed more dispersedly. Wherein, Cas13d was mainly distributed in animal microorganisms, which was consistent with the Ruminococcus genus that was previously found to be mainly distributed in Gram-positive bacteria. Corresponding to the novel Cas isoform, Cas13i was distributed in humans or animals, and Cas13g was mainly distributed in methane-rich environments (FIG. 9). At the same time, among all novel Cas13s (1139), Cas13a, Cas13b, and Cas13d accounted for 23.7%, 46.2%, and 9.5% of the total, respectively (FIG. 10).

### Example 3: Identification of Novel Cas13 Isoform crRNA Directions

### Plasmid Construction

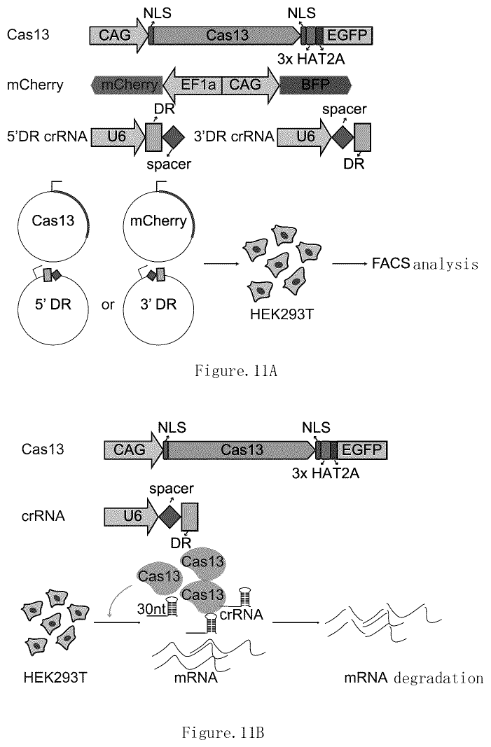

The coding sequence of Cas13 was codon optimized (human) and synthesized. The synthesized Cas13 effector protein sequence was inserted into the XmaI and NheI restriction enzyme sites of the pCAG-2A-eGFP vector to construct the pCAG-Cas13-2A-eGFP plasmid. The protein sequences used were shown in Table 2. The synthesized crRNA sequence (U6 promoter sequence+DR sequence+spacer sequence or U6 promoter sequence+spacer sequence+DR sequence) was inserted between the EcoRI and HindIII restriction enzyme sites of the pUC19-U6 vector to construct pUC19-U6-5′-DR crRNA or pUC19-U6-3′-DR crRNA plasmid. The corresponding crRNA targeted mCherry mRNA, and the mCherry spacer sequence information was shown in Table 3. The CAG promoter expressed eBFP protein and the EF1a promoter expressed mCherry protein to construct pCAG-eBFP-EF1a-mCherry plasmid (FIG. 11A).

TABLE 2

| SEQ | | |
|---|---|---|
| ID | | |
| NO | Cas enzyme | Amino acid sequence |
| 1 | Cas13bt1-2 | MEKEQGLYSIDRYQGAGKWCFAIGANRAWDNYNERPKLFSESLL |
| | | RYEKATRRDWFDEETRGLIKKSDVRQRLRKIRCYFSHYCHDNTCL |
| | | GFDPDDDLRKIMEKAYERAIFEQRKHLSTETDIETPALFEPHGRITA |
| | | AGVVFFCSFFVERRILNRLMGRIPGFKKTEGEYGATRQMFSKYCL |
| | | RDSYSIRASDSNAVLFRDILGYLSRAPSQYYRHNKDQCDKDGHPE |
| | | RKKDKFINLALRYLESFVPARLRNHTLSVGRKEVVRMETNAVAE |
| | | GEGEYRPYPPKAKVKVVFTEDDPERPYYITHNTVILQTAKKEEDIH |
| | | HCKVGVNELKYLVLLCLQGKAEKAVAGIEGYVRRIQGRFADHTN |
| | | KVARDDDERLVRGLPEFVRVASGIETPDEVRELKSRLDHIRKKWQ |
| | | TKKAESAEAQLHRKARDVLRHINWESQRPLGIEQYNRLLELLVNR |
| | | DLESFAAEMKELKRRGLISEELLKSVEGIRNLNTLHVKVCNLVLTR |
| | | LEHLVENDPEELKRHIGIVPREEKEGPSYEEKVRAFVQQPMMYRG |
| | | FLRNSFFKGSGKSFAKLVEEELHKKGCPDVPLGTDYYLVRDLERD |
| | | ERKNRFHNDNAALYETLALDRLCVLMARDCLVRLNRNLEKHAT |
| | | RISWEATDAGDTICLELPRRDRDHESFRLSFGVRDYPKLYVMDDP |
| | | VFLCGLMKHFFPDNQAIQYHELYSEGINKYTAMQAEGIAATLKLE |
| | | EKTIKEKNMQIPATGYIRFCEIVSQSDFAPGEKRVLKNVRNGLLHY |
| | | HLEFEPTEWAEFREIMKREGFDTAKKRKSTRKK |
| 2 | Cas13bt1-5 | MKIKNENTDKKTELYSIDKYKGRDKWCFAIVLNKAQTNLTENPD |
| | | LFEQTITKYDRIRKEGWFDEETKKLIYIQENEHKIKGEIKTLAREVL |
| | | KNLRNYFSHHFYKQDCLIFPKDNIVRIIMGRAYERSEYEIKKNLKE |
| | | DISIELPALFEPEGKITTAGIVFFTSFFVERRFLHRLMGSVQGFTKTE |
| | | GEYKITRDVFAKYCLRDSYSVKAQDNDAVMFRDIIGYLSRVPTES |
| | | FQHIKNPKKQNESQLSERKTDKFISFALKYLKDYGFEDLKGHYTA |
| | | FFARSEIKKEKEDIEIKDDKKHKPHRMKSKIEIHFDKTKEDRFYIER |
| | | NNVILKIQRKGGRANILRMGIYELKYLVLLCLSGKAREAINRIDDY |
| | | LNDLRNKIPHIENMNKEGIGEQIRSLPGFVRSQLGFVQIDDEKKKE |
| | | NRLDYVEKKWEKKRAESKELKLNRKGRDILRYINERCKKPLTIDR |
| | | YNRILELLVEKNIEGFYHELEELRKTGRIEKNITQALVGEKNINALH |
| | | IKICKLVQDELKSLEKEDLKKYIGLTPKEEKVVSFEEKLGRILDKPV |
| | | IYKGFLRYQFFKNDKKSFARLVEEIIKEKTGGLDVPIETEYYSISTL |
| | | GRFDKANKTLYETLAMDRLCMMMARRYFLSLNKILAKRAQNIE |
| | | WKKESGKEFIVFKFNMPQDTGKSISIRFSPKDYTKLYVKNDSEFLA |
| | | RLCQYFFPNEKAIDYHKLYSHGINKYTNFQKEGIEAILELEEKIIKK |
| | | RKINSPENYLSFEEILNQSIYNDEEKNTLIQIRHSLLHYQILFSKNDL |
| | | TKFYNVMKREGIEKIWSLVI |
| 3 | Cas13bt1-8 | MAVDYSLKQPFYQGVHKSCFTVPLNIAADNCKQKGYRNLLKEAQ |
| | | RSKGGLSDQSIQEAADLIEKRLSAIRNYFSHTYHTDSVLTFQKEDP |
| | | VKKFLETAWSYAVSETQKDIAESDYTGIVPPLFEDKEGQFQITAAG |
| | | VIFLMSFFCHRSVLNRMFGSVKGLKRSDREQMGTGEKRDYQFTR |
| | | KLLSFYSLRDSYAVKAEATRPFREILSYLSCVPHESLVWLSARGKL |
| | | TEKEKKAFRHFLDPTVPKEALSEESAGDGSDSERPGVRKNNKFLL |
| | | FAVQFIEAWSRKEKKGLEFARYRKSRVEAPGENQDGSEKRIVRFR |
| | | SEIRDTQEDWPYYLRNNHALLRLHPGENKEPVDARIGEYELLYLV |
| | | LAIFDGKGAKAIQKLANYIFEAKKQIQNARVYDRYQDLLPSFLTA |
| | | GNKPVSAETIRNRLAYIRGELEKMLEAVQKEKKSGRWEMHKGKK |
| | | IGHILRFLSNSIDDIRRRPNVKEYNRLRDLLQQLQWDEFDKALQSY |
| | | VNEKLLDETVYRQLRGFHSLDELFERCCRLELKRLEDMEKAGGD |
| | | RLNRYIGLEPKGKPKNYADLNTLQKKGERFLKGHQLSIPRYFLRN |
| | | ALYKEYQATEERKPTSLYQIVRERLPRTNPILPDRYYLLEEDPKTY |
| | | SGSDSKIIREMCFTYIEDLLCMRMARWHYEQLSEKLRKKLQWKE |
| | | VQTGPAGYERFRLIYKISDELSIEFHPSDLTRLDVIEKDDMLTNISQ |
| | | HFLTKKGTVRWTEFVSQGMKHYRDRQKQGIEALFKWEESLRIPE |
| | | GLWKEEGYLGFEKVLEEAVKHGKIQDKDKEALKRIRNDFFHEHF |
| | | CGTPADWEVFKRVLKRFLNQGKNEKKRFKK |
| 4 | Cas13bt1-9 | MPVNYSLDQDYYKGTHKSCFTVPLNIAWDNGSKKGCENLLKEA |
| | | MRTRGGFTQEDIEKVHRSLAEKLNGIRDYFSHYYHEDKPLEFKKG |
| | | DDDAVKDFLEKTFSYAAGETQKRVKESGYQGIIPPIFELCGDQVRI |
| | | TAAGVIFLASFFVPRSTLERMFGAVQGFKRSDRGDLDTGQKRDYY |
| | | FTRSLLSFYTLRDSYYLQADETRPFREILSYLSCVPFDSVQWLQAH |
| | | GKLSKSEEKEFFGRPVEEQDEENPAQTEKQTAPAGRRMRKKNKFI |
| | | LFAVRFIEAWARNEKLSVEFGRYRNIQNEEDRRKQSGKKVREVFF |
| | | PSALNNLSAEEQDLEGLLYIRNNHALIRIHLKAKTPVTVRISEHEL |
| | | MYLVLAILSGKGGNAVQKLSKYVWDVRMRSRGPLTNMPRNFPA |
| | | FLRSPASEVSEQAVQNRLNYIRKTLKEIQANLQKEAQTGQWILDK |
| | | GQKIRHILRFISDSMPDFRRRPSVKEYNELRELLQTLAFDDFYRKL |
| | | ASFQTERKLDAAVWNNLAQCKSINELCERCCQLQQQRLDELEKQ |
| | | GGDELKRYIGLLPKEKGKHYEEQNTPARKFERFIENQLSVPKYFLR |
| | | CKLFVTGGSRRTNLLKLVQEHLKPKTSVFHEERLYLREEQPGDYP |
| | | WSDRKIIQKMYYLYVQDLLCMQMAQWHYEHLTPQVKGKIDWEI |
| | | NSESKESDGYNRFKVEYKGPQGCRIIFRVQDFGRLDFLNKAPMLD |
| | | NICQWFLSGRKEITWPEFLRDGLQRYRQRQILVVRALFRFEENLKI |
| | | PEEEWKGKSHLSFDEVLERFSGKNRLSEEEKESIRRVRNDFFHEEF |
| | | EATPSQWRDFERRMSEYLNKEKREKPKKKKR |
| 5 | Cas13bt1-10 | MIENKPISQGSPQGKTVDDYKGEQKWCFAIVLNRACDNYEENHK |
| | | LFSESLLEFEKTHRKDWLDEETRKLIHNVEEILPPDPQKKYEIKPKN |
| | | LANIRLNEVRNYFSHFHHKDWCLYFKADDPIRIIMEKAYDKAKEK |
| | | VIGHLKKEPEIKIPESLFESNGRITPAGIIFLASFFVERRFLSRLMGYV |
| | | GGFKESEGEYSITRDIFSTYCLKDSYSIHTPDSKAMLFRDILGYLSL |
| | | VPSEYYPTYLSQIPKRKPDEKLPKDEKYGERKPDKFILFALKYLEEI |
| | | VSKGLADTYKVSVARMEIIREETKEAEKSDEQYKPRPNEGTVKIV |
| | | FESKSKPDGEELPYYINHNTVILRIQKKGDKIHFCKMGVNELKYFI |
| | | LLCLQGKTAEAVAAVDNYIHSLQSRFANPAETVRSDEAGIPEFILR |
| | | QSGKVQDKDKEKAARIKYIRDKWEKKKAESAEMELHRKGRDILR |
| | | YVNWNSKQPLGTNKYNLLLELLVKKDFDGFGKQLFNLKLKEHIS |
| | | DEVFKRLTAFKTINTLHEKVCSFVLEELTFLEQNEPAKLEEYIGLV |
| | | RKPAPENNPPPEYKDKVKAFVEQPMIYKGFLRENVFKENKKTFAK |
| | | LVEETLGRLKYPDVPLGKDFYYVVDPKLSEKENRFQKDNKILYET |
| | | LALDRLCAMMARICYENINENLRKSGQEIIWKKENDKEFLYLSINP |
| | | AKLTTATLREPKTSRTAFGDNLKIPLPSGTQNTFTIRFDRKDYTKL |
| | | YVMDDAEFLGGLVLHFFSKEKEPIDYHRLYSEGINYYTELQRQGI |
| | | MAILKMEEKIVMNKKIPMTGNYIGFKTIMKESGYPPLEQNTLNKV |
| | | RNALLHYHLKFEPTDYNKVVEIMKREGLESKNKIRKTDKK |
| 6 | Cas13bt1-11 | MGNISGEKIGIKMDNKKKGNNYSIENYKEDRFLFTAALNIAYDNC |
| | | KQKGCLNILAECQHSKGGISDEQIKNVKDGIESRLRDIRNYFSHYY |
| | | HNENCLMFEKDDPIKVFMEATFDKAVSNLSGSTKESDYKGIEPEQ |
| | | LRLFEEYDKKYRITMPGVVFLASFFCHRSNVNRMMGAIKGLKRA |
| | | DRAEMDDGTKRDYNFTRRLLSYYSLRDSYAVKNEETRPFREILGY |
| | | LSLVPHEAVDWLDSRGELSNEEKKEFLKEAKNQESKEDNDSTDE |
| | | KTRRGLRKGNKFMRFAIMFTEDWSKKENLEVTFARYEKQEVHLE |
| | | NKKQDGKKERNIKFPHEISASDDDWPYYIRNNHAIIRIKLKDKDAV |
| | | SARISENELKYLVLLIFENKGKEAIQKLGDYIFDMSQKIRYDNYEP |
| | | KDARRIPSFLKITRKEPTYEEVNNRLTHIRRELGKIIETIEKELKESK |
| | | WLIYKGKKITIILKFLSSSIADIKKRFNVEQHDALRDMLQKLKFDEF |
| | | YKRLSSYVGDGTLDKKTYESIQGIKDISQLCKKACELRLARLDELE |
| | | KNGGSVLYRYIGLEAEEKNKEYEKLNTNQAKAERFLESQFSTGKD |
| | | FLRESFYEQEREQKKSLIKIVKEQFANVVPMNEERWYLMNKNPK |
| | | KFKDKDNKAIKALCNTYVQDILCMKIARWYYEGLSHAYKDKIEW |
| | | DSTVETGGCGYTRFRLNYKTDCGVVIEFKPSDFTRLDIIEKPKMVE |
| | | NICRSFITSNNDKKRTISWYDFNKEGVTKYRKQQVKAIERIFAFEK |
| | | GLKIQDEKWQVQGYVPFIKRPEYENKGFKTFILEDAIQQSKIAEAD |
| | | KETLNKVRKDYFHEQFFSSDEDRKVFEKCMPVVDDKKKFGKKNN |
| | | RMYGKKG |
| 7 | Cas13bt1-14 | MIENKPISQGSPQGKTVDDYKGEQKWCFAIVLNRACDNYEENHK |
| | | LFSESLLEFEKTHRKDWLDEETRKLIHNVEEILPPDPQKKYEIKPKN |
| | | LANTRLNEVRNYFSHFHHKDWCLYFKADDPIRIIMEKAYDKAKE |
| | | KVIGHLKKEPEIKIPESLFESNGRITPAGIIFLASFFVERRFLSRLMGY |
| | | VGGFKESEGEYSITRDIFSTYCLKDSYSIHTPDSKAMLFRDILGYLS |
| | | LVPSEYYPTYLSQIPKRKPDEKLPKDEKYGERKPDKFILFALKYLE |
| | | EIVSKGLADTYKVSVARMEIIREETKEAEKSDEQYKPRPNEGTVKI |
| | | VFESKFKPDGEELPYYINHNTVILRIQKKGDKIHFCKMGVNELKYF |
| | | VLLCLQGKTAEAVAAVDNYIHSLQSRFANPAETVRSDEAGIPEFIL |
| | | RQSGKVQDKDKEKAARIKYIRDKWEKKKAESAEMELHRKGRDIL |
| | | RYVNWNSKQPLGTNQYNLLLELLVKKDFDGFGKKLFNLKLKEHI |
| | | SDEVFKRLTDFKTIDTLHEKVCNLVLEELTFLEQNEPSKLEEYIGL |
| | | VRKPAPENNPPPEYKDKVEAFVKQPMIYKGFLRENVFKENKKTFA |
| | | KLVEETLGRLKYPDVPLGKDFYYVVDPKLSEKENRFQKDNKILYE |
| | | TLALDRLCVMMARVCFQQINENLVQRAEQIDWKKENGKEFIYLSI |
| | | NPAKLVIAQTQEPKTSRTAFGDNLKIPLPSGTQNTFTIRFDRKDYT |
| | | KLYVMDDAEFLNGLMQYFFPKEKTIDYHKLYSEGINHYTELQRQ |
| | | GITAILVLEKKIIDREKLPTDVKYIDFRTIMENSGYKREEQIALGQV |
| | | RNALLHYHLGVEPKENNKGYKGFKPDDFKTFVAVMAREGIRKRE |
| | | KWNLKI |
| 8 | Cas13bt1-15 | MGIDYSLTSDCYRGINKSCFAVALNIAYDNCDHKGCRTLLSEVLR |
| | | SKGGISDEQIKSQVVDGIQKRLKDIRNYFSHYYHAEDCLRFGDQD |
| | | AVKVFLEEIYKNAESKTVGATKESDYKGVVPPLFELHNGTYMITA |
| | | AGVIFLASFFCHRSNVYRMLGAVKGFKHTGKEQLSDGQKRDYGF |
| | | TRRLLAYYALRDSYSVGAEDKTRCFREILSYLSRVPQLAVDWLNE |
| | | QQLLTPEEKEAFLNQPAEDEGGDISDSSSSDKNKKSKEKRRSLRRD |
| | | EKFILFAIQFIEGWAAEQGLDVTFARYQKTVEKAENKNQDGKQAR |
| | | AVQLKYRNQGLNPDFNNEWMYYIQNEHAIIQIKLNNKKAVAARIS |
| | | ENELKYLVLLIFEEKGNDAVQKLNCYIYSMSQKIEGEWKHRPEDE |
| | | RWMPSFTKRADRTVTPEAVQSRLSYIRKQLQETIEKIGQEEPRNNK |
| | | WLIYKGKKISMILKFISDSIRDIQRRPNVKQYHILRDALQRLDFDGF |
| | | YKELQNYVNDGRIAVSLYDQIKGVNDISGLCKKVCELTLERLAGL |
| | | EAKNGSELRRYIGLEAQEKHPKYGEWNTLQEKAKRFLESQFSIGK |
| | | NFLRKMFYGDCCQKRCFDEEKGYNTQAKERKSLYSIVKEKLKDI |
| | | KPIHDDRWYLIDRNPKNYDNKHSRIIRQMCNTYIQDVLCMKMAM |
| | | WHYEKLISATEFRNKLEWNCIGQGNMGYERYSLWYKTGCGVVIQ |
| | | FTPADFLRLDIIEKPAMIENICQCFVLGNKKLNSGAEKKITWDKFN |
| | | KDGIAKYRKRQAEAVRAIFAFEEGLKIQEDKWSHERYFPFCNILDE |
| | | AVKQGKIKDTGKDKEALNRGRNDFFHEEFKSTEDQQAIFQKYFPI |
| | | VERKDDTKKRRDKKQK |
| 9 | Cas13bt1-16 | MAVNYSLREKWYRGVNKCCFTVALNIAVDNCKSKGCETLLKEA |
| | | EHSKGGITDEQIQQSYTEVEKRLNDIRNYFSHFYHGDECLIFKKDD |
| | | IVKRFMESVFATAVSNVVGGTKESDYKGVVPPLFEQSNEDYMITA |
| | | AGVIFLASFFCHRSNVYRMLGAVKGFKHTGKEELSDGQKRDHGF |
| | | THRLLAHYSLRDSYSVKIEETKSFRDLLGYLSRVPQQAVDWLNER |
| | | NELSEDEKKEFLNQKSSEEESPEQPEPENAEWRTEKTSRRSLRKTE |
| | | KFILFAAKFIEDRAEKEKQDVTFARYQKTVTKEENKNQDGKQAR |
| | | VVRLKYEEDKKDDEKPREHFNLEWMYYIRNEHAIIQIKPKDKEAV |
| | | AARISENELKYLVLLIFEGKGGDAFNKLSDYIFRMTQKIKSGQINP |
| | | NEARLPSFLKNPVKNITDKMVRNRLDYIRGQIKDVLEKINMEEPQ |
| | | NNKWLIYKGKKISLVLKFISDGISDIKKRPNVKEYDTLRDTLQKLD |
| | | FNRFYERLKSYVSDGRLAAALYDKIKGIDDISELCKKVCELMFAR |
| | | LAELEKKGGFELYRYIGMEVQEKDEKYDEWNSPQKKAERFLESQ |
| | | FSIGENFLRESFYSEYCQKQECIDKEISLNTSVKNRKSLVYIVKEKL |
| | | KDIMPLHNDRWYLIDRNPKDFERKDSKVIKGLCNTYVQDVLCMK |
| | | MARWYYGQLNPALKNNIKWDETGQGHGYDRYKLSYRTNFGITIE |
| | | FKLADFTRLDIIEKSDMIENICRSFIKPNRTISWYDFKQDGVEEYRK |
| | | RQYKAVRAVFAFEESLIIPGRDWLSQGFVPFIKNEEYVKKGFSLFV |
| | | LDEAVRQLKIKGSDKDAMRQVRNDFFHEQFQAKDEQWKVFEGY |
| | | LSCFMIDRPKGEKNKKRYNGNKK |
| 10 | Cas13bt2-5 | MNMDTIELKKEEAAFYFNQAGFNLRAIESNVFDAGKRKTLLENPQ |
| | | VLAKLENFIFNFRDVTKNEKGEIDVLISKLTDLRNYYSHYVHTDN |
| | | VKVLSKGEGPILARYYQFATEATASTNVKLEIMDKGNKLTDAGV |
| | | LFFLCMFLKKSQANKLISSISGFKRTDAEGQPRRNLFTYFSVREGY |
| | | KVVPEMQKHFLLFDLVNHLSNQDEYIEKSQQTYDIGEGLFFHRMA |
| | | SKFLNTSGILRGMKFYAYQSKRLEEKRGKIEPEGDSFVWIEPFQGN |
| | | SYFEVDGHKGVIGEDELKDLCYALLVAGKNANEAEGKITQFLTK |
| | | YKKADNSQEIEKDEMLCIDNFPANYFDGPGVGSIKDRVLNRLEREI |
| | | KSHKDNKADSKAYDKMKEVMDFINNRLPAAEKMKQKDYRRYL |
| | | KMVRLWNREKGNIEREFKAKEWSKYFPSNFWRANNLEDVYKLA |
| | | RQENARILGNLKAVVEGMSEQEFEKYRQINEAKDLAGLRQLAGSF |
| | | GVKWEEKDWEEYARQIKERITDRQKLTIMKQRITAELKKRHAIEN |
| | | LNLRITIDSSKSRAAVLNRIALPKGFVKTHILQTPSDKIMKKTREAE |
| | | CKILLSGKYGDLSKRFFDEKNLDKLTQINGLYEKNKIIAFMVVYL |
| | | MEQLKLGLKGKTKLAELKETRIRYKISDKVTEDIQLSQYPSLVYVI |
| | | GRKYIDNVDRYKFAGDVRGKPILSKIDLIEKERLEFIRQVLGFEKEL |
| | | FDNTVIDKKNFTDTETHIKFRQIIDELVRKGWDATKLNKLKEARN |
| | | AALHGEIPEGTSFQEANVLINELKNKK |
| 11 | Cas13g1 | MDICGRDVFLKRYGGACKNQEERKKQQKFFNEIKNFLKTLRNYFS |
| | | HYLHTKEKVESLLKEEKCREFVSFLRRKALEVWNKRFENSALFSE |
| | | MQAFSQNGWEEMKRELKESGPFLTDVLLFVSVFVPRGEMMRLLD |
| | | AFRLSPDREKRECRREILSALCLSESYDFWSVDKKAGIALTILDHF |
| | | ARIMGKRTDKAGKLIMSAPRKPENRKETPEICRRFRPSFFIRMLVG |
| | | FIEEENVLKGFEFARSYNGRPVYDESKKELYLCRNNTRVRFPDNE |
| | | GNLLESELGAEALKRIVLLYLKNLKGNDLAPLVRGKIKAIQKTLPQ |
| | | RKNGTNAALLQRVKSRRDELSIRFFRKGLSDHQKAVAVLDFMNLI |
| | | FPADKKFGKQAYQEALRALTGRYDEEAFLAVLPDIKCSETPFPKR |
| | | KKPQAWARGAGSLETLHKRAEALFREKAGSLCEETAEEIGRLFHV |
| | | RALKEKTGEPFERFKKLLTPPPRAFTGNGFGLTKEELLKDIPLEAEL |
| | | YAPGGGPMASAVKKIYDNDRLLLKLARFILEQIREKDKVQVQITE |
| | | KPDYDYEALVTVGEKFQVIIDSAQARRSHVSLNTERLKNVIQNYW |
| | | KREETPVPFVVPDMRKREKNTPPRCWTEAEQDMERERRTMVEAL |
| | | LKWEKYLANEFAKENKTATGNIDAIIKLMKPEKGEYVDFPLLLAR |
| | | MNIPEASEYNDFRKWAYHQAPKKAFSDAPGELKERFLKRQAEKD |
| | | RKRREQKKNSIKKVQKK |
| 12 | Cas13g2 | MNKRAENKINFFKDNFTLGQYAAFALSFNQAGRQLEKLSTTYHF |
| | | QRIYGRRRDKVPSFKESLKNLRNYFSHCASQWPDEERFKKSVLRN |
| | | ELDFLVLEAIAILEQRNTKLHDNDETAKDIKKELDKIRSSPSDFFPL |
| | | DNSGKNIQPTLALVLSLFLTKNQMAFLLGKIFRGNGVTRESPQYL |
| | | AQAKILETLSQNDRTLVDTDSQSERFTSHDKEFGLAIAGRLEAVGL |
| | | YEEQQKIEDFPEDVWFIKQLVLYLEYMNVLPSVQFCRMTTIEKDD |
| | | QLLQEKDFDIAHREKPLRIRCNTVEAQVKLSNGAPYTTNFGVQSL |
| | | KYLVLAHLKKTFEDKEIDQLVVRQIESNPSRKCRKAKESGVTGDR |
| | | LTKRIEYLIGKHTPSGENPVRLYEQIRFICRFVNEAWFKRYNRHMN |
| | | SEEFKDIQERVRHYRRDDFQKLLKESDLLDIAGLNLGAGNDKKLG |
| | | SCFRHERIQNIFCEMESGYLEWLENRKQQIDKLSQEEREALAARIN |
| | | LRHREGRSSQEAFRPVSISADVLRREIESRDKAKNKRRFFDHIRDF |
| | | GGSARFFARFDLDDRGWGSARDRERWARTGLLSQIVLKSLSGANI |
| | | KNMLDQKPSQWECEERVDGTKITFKLTQGWRYYAATTKTQLRK |
| | | LIGAYRPDLSVLPLLDDGGIEGGSVQSLKRTLERERYRLMQAILV |
| | | WEREIIEKHDMKPDGGYIKFESILEKGATAEQAPDLKDIRNHCMH |
| | | GDIWKAPFSQAPEPLRRVYSGLEEKSREKRRSQKKSGIKRSQKNQ |
| | | K |
| 13 | Cas13g3 | MRGGNKSANEGKKFDFNLKQQAIFALGSNWAQKQFELTKKTQHS |
| | | HRLREKLSIFQEGDIGRLEEKIENLRNYVSHGAHSGLAPLDSDEIA |
| | | AFEAIVRKAVTSYLAIPREAQNIKEEKQTKIDKIRARMKKDKPLLS |
| | | FSTGPLHQQNHPELMFLLAHFLTRKQLSYLIHRVYWPKEREDDKE |
| | | PIQELLLFIAQPDTIIMRSKADEDARDTWISAEEEQGFAIWNYLQK |
| | | RHADEVYTAPDDHYVMRQLVAFIESHQILKGAIFMRVEVRPDEK |
| | | KEGAYKRVGVYEKQGSDNSLPLNIAYNTIRVSFVEDKVEGTFSLK |
| | | TLIYITALFIGRVTPDKLTDFLITELKKNRDYSHRPSPAAKEGGDIK |
| | | TRVEKRLCYLLRRLNKPPQNLQEQIRFICQRINFAYQQKYGQYLD |
| | | QNDYKTLENLVRYYRKPDLLSWLEGNAIGQQSGIHMGQEDSKTL |
| | | NQLIKASSIEQLYLDMKSHYRYGLKYVAKNYQNWPEDKVADLA |
| | | NIIGVRQQKTVSGNLPNAPVGVKLSWILTEFQDEIEEFKGIIHMLSA |
| | | QYAPFKFEKPDKKFKKPGDAPRSEKNKRGWAHAKPFVVKQLLLN |
| | | MAWYNVKEISDNDARLAGGAFVPISDISFKRSFSGCSLRMSFGKS |
| | | WRQHARKSREYLQGLIDSYGEGRREFVLSKAEEENGISKSIERMEE |
| | | EARQERLLFIQAILDWEKGWLKKNKSEAERLKTAKGYVEFREIAE |
| | | KESLPDTIKELRNKAFHDGFLRDTKFSDCVEPIKSIYEDLKQKHI |
| 14 | Cas13g4 | MSEDLYRSDNLFCLSLEQQAALSIALNVALRRCREQNDSDMDICG |
| | | RDVFLKRYGGACKNQEECKKQLDFINEIKGFLKTLRNYFSHYLHT |
| | | KKKVESLLEKEKFRDFVSFLRLKALEIWNKRFGKSSLHSQMQAFS |
| | | QNGWEEMERELKERGPFLTDVLLFVSVFVPRGEMMRLLDAFRLS |
| | | PDRKKRELRREILSALCLPESYDFWSVSKKAGIALSILDHFARIMG |
| | | KRTDKAGKLIMSAPRKPENRKETPEICRRFRPSFFIRMLVGFIEEEN |
| | | VLEGFEFARSYNGRPVYDESKKELYICRNNTRVRFPDNEGKLSESE |
| | | LGAEALKQIVLLYLKRKQGAFKGNDLAPLVRGKIKAIQKTLPQRK |
| | | NGTNAALLQRVKSRRDELSIRFFRKGLSDHQKAVAVLDFMNLIFP |
| | | ADKKFGKQAYQEALRALTGRYDEEAFLAVLPDIKCSETPFPKRKK |
| | | PQAWARGAGSLETLHKRAEALFREKAGSLCEETAEEIGRLFHVRA |
| | | LKEKTGEPFERFKKLLTPPPRAFTGNGFGLTKEELLKDIPLEAELY |
| | | APGGGPMASAVKKIYDNDRLLLKLARFILEQIREKDKVQVQITEK |
| | | PDYDYEALVTVGEKFQVIIDSAQARRSHVSLNTERLKNVIQNYWK |
| | | REETPVPFVVPDMRKREKNDPPRCWTEAEQDMERERRTMVETLL |
| | | EWEKYLANEFAKENKTATGNIDAIIELMKPKKEGYVDFLSLLERM |
| | | KIPEASEYNNFRNWAYHQAPGEAFSDAPGELKVRFLKRQAKKDR |
| | | KRREQKKNSIKKVQKK |
| 15 | Cas13g5 | MIDSFKDGFTLKQFVAFTLAANWAFDQHNNDQHNNLINTQHFYN |
| | | LVKKNMKFKGDEDIFRGEEEAESDAPPKSIIKSIRNYFSHFIHSPLRS |
| | | LTTVEARDLNDMARRLLVELNERNIKANDKDDIQQHIETYLDEST |
| | | SLFTFEPKPILDHAQPEMAFMLALFLSKGQMAFLCGKIFFGTDARS |
| | | KQKDGTYKDTPKTCLQKKLLNMMAQPDNIIRRLQSDDTLDPWLE |
| | | SKHEQGFAIWQLLASSQKETAGGNEPDYLKNGNYMIRQLVRFIEL |
| | | HNVLPSYEFARIETVETLSDDVKKLEQKIQFSSNPDLPLATRHNTIQ |
| | | SVNQQGIKGTFGVRTLIYIVVVFLEQKRAGELANFVTTWLKANAH |
| | | YPKTNLKGKDRPLSDQIAKRLDYLLKETNVKSKTNAKKSLHMQIR |
| | | FICQRINHVWSLKYERHLSVHEYGELEKMVRYYRKAELRGWLAD |
| | | HGLLDIQNIQLGRGGIKTLKKAIKAESIQQLYVDMLSDYHCWLQE |
| | | QKDKLPKLNDAAQQSMAKAINVRNNRQSNNKPDFPIGLPEKVLR |
| | | QKFFVVDGKSHARSLTTILKSIPTPVTFNEPQGTKSKALKKSMQIR |
| | | NYQQLLLAMAWHAVKDLISDERIKQEKNSETLPDLAEIPISINCEN |
| | | VRISMKFKKSWRNMATLNKGYIGKLMKQYCAGEKTIPMFRADET |
| | | KAGKSVETTMQTFHSERYRFMQALMQWEKEFYNTNPALAESSDE |
| | | IYLKFECLAEKANLADGIVSMRGDAFHDGVPNARFADCPEPTIKTI |
| | | YDQICEREKDKQKTQRQQGVKRNLAAKKNADKS |
| 16 | Cas13g6 | MNHDVAHSHIKDDVTLRQAAAFTLASNWAHQQHKGVLASVHGE |
| | | SLYRYALPVDEETIAAYRNHFAHLHRPHMPRLWQEPDVKDRLHT |
| | | LVKEAARMMTMREGKIWDDDEKQHNAIHTILLRIENDPAYGFDL |
| | | DRPQQAWLSITLILAPFLQKGQMAFLWDKMVEKRGDADEALDK |
| | | ARFHLLKFLSIADSALAITPQQEEERLFSAQTEHGLALLVRLKESLD |
| | | KITQDESSQATHAFPQGGYVMRQLILFLERTNALPSVMFARTRTH |
| | | LEGERDGKPLLRQERIFVRERDAQTHDGMPLPPLKIQANTIRVQIQ |
| | | EPKTDKQWQGMLGIHTLSVLVCAFLLGKDVDRLVLSWYREHGD |
| | | KNDWRQGKEAKSPTPERIGKRIDWHRERMEGATTLYQKIRMLAD |
| | | LMHESYREKHGVAMSTSDYRDLLYHMRHYRLHTLKAMARQDY |
| | | GIETMNVPMTGKPWSFLLKKQDLDAQYDDIVAYRCRWLSDVKE |
| | | RCHRMDDKERMTLARALGVRDVSPKDVNARGKNRVALLPAGMP |
| | | VDAVRDAFPEFRAGHPDDKHAKKRHFIDVMRDNKTLKQGGGIPT |
| | | QPFGFTWDTDKANKDKADKDKTCAGGWRAMTKRRSQWAHASL |
| | | LHMMARHLLQGDYAVETPHDKKGKATSHESNKKKQVALGEMPF |
| | | ASVKPSALGMTLEVEQGKKVLCSLTQGWRYYARLDKKTIKNLVT |
| | | AYTKAGKGRTWTLPLLRASAGDKEESVEQAVTEMRRQRFIAAQA |
| | | ILQWEYDVLKGKDIDKDKRLDFPAILTYAGLHDDSIRKKLTHYRD |
| | | YVFHDNVLEHPFRDAPDILRECYKKIEAEEKKRRQQKRRQHIHQR |
| | | PVQHSGKGKGSKKHKKTR |
| 17 | Cas13g7 | MQDRQVKREPFKDGFYLKEYAAFAIAAHNAHLACIKCATSILETE |
| | | FFAKRYFPLLKAAIAHHPDFTPNAGQASWPQREELASFTKSDRAQ |
| | | KYNQAFSKCLTEYAGGIRNYFAHYLHTLKPLQLPAEDRVFCFFLE |
| | | ELQKKAVAVWKARHAAWRAKQSPHGEQYDSILECSQRPDFWDP |
| | | LTVQRDDCTILSCEAILIFLSILLPRSRMYMLLDKVLVRGESGTETE |
| | | KKRISKRELLTALSPRDGHSIRIDPERKSLTAAFTVFNHIGTRYGRL |
| | | TDTDGNLLSPRPNNIDDIVASHHTVFFIKMLVAFIEGEKCLPSFEFA |
| | | RSYHGKPVFDQPDRELYIRHNNVAVRLRDEPRQQAVFSIHLLKLII |
| | | LNLLSDKGNQTPDVDDWLKKQLRNFRSRAGQGDHSATPNSNLER |
| | | MTPSFMKGEPGRPPKQACSLRKIVENRLQLLQERWFGENAETLLP |
| | | HQMAYAILRAVNLMVPPENVLNSLQFREALLALTAIPYSADAVK |
| | | GALPDIPRRKLNVGGQTLRGRLSGSQNINAAYGKIRSGFSEYCNE |
| | | MMDLFDDQETTALRTFAARIGVQYIRKENSLEHERHKKDREQYQ |
| | | KHYLEKLVSIPPVLFVRQFMDDDAKNVCFGTLLQRSRYIREVREA |
| | | GLWDVKTLLADGQKFRDKHKEVINRDQLLLALVQYVQERNLKA |
| | | QIKTGKKKKQGNITSLFDLSEWKSSFAFRVEGGKSVVVDTAKAW |
| | | KYYLRTDPAWLQKVFKTYCSEIQTDTLPFVGPPDLPRRKENTPPRS |
| | | WREAEGDMEQERYILMRSLLIWEKNTGSSRAKGEEYKPFKDLVT |
| | | EAALPNGDEVTSIRNAAFHGVPDKPFRDAPEPLQQIYQQERERLQ |
| | | KKRGEKKRKAQKN |
| 18 | Cas13h1 | MENTSLRTFKRFFDFKGSVAPIAEKANRNYALKKRNNVNLQQRL |
| | | HYFAVGHAFKNIDVENIFRAELDEETKGKKPTKFLALQLSNFTFIK |
| | | ELGCLLSNIRNINSHFIHDFELIKLDKIDDKIIEFLKQSFYLAVIQTCI |
| | | KEKETTYIDFISTKDYEKQIVNFLLEKFYPFNDKRKKLTEEEEKRV |
| | | KEYKSFRNDFKNKSIDEAIDSILFVKVNETVEWNLFETHNVFNITS |
| | | GKYLSFEACLFLLTMFLYKGEANQLISKIKGFKRNDDDKYRSKRN |
| | | LFSFFSKKFSSQDIDSEENHLVKFRDLIQYLNHYPTPWNKDLELES |
| | | ANPAMTNKLKEKIIEMEIYRSFPDFANDERFFVFAKYQIFGKKYLG |
| | | ENIEKEYMDNSFTAAEKIAYEYEIKISPEIKDANNKLKELKAKQGP |
| | | YGKQKERNEKIIRELESMIKDGKNDPNPITEKLKTRIVKNLLYVSY |
| | | GRNQDRFMDFATRFLAEEKYFGEDAEFKMYKFFSSEEQENNISTL |
| | | KKELSKKQYDNLKFHQGKLVHFCTFDEHLKNYESWDDPFVIENN |
| | | AVQVKVYLSNGINKIVSIQQNLMIYFLEDALYCNENVKELGKTLL |
| | | TDYYLMHKEEFEHTKLFLQQNPTISREDKTVFKKILPKRLLHHYSP |
| | | AMQNNLPEFSTFQLLFEKAKRLEERYNKLKNKAEDEGNLDDFLK |
| | | RNKGRQFKLQFIRKACHLMYFKESYLRQVQEAGHHKRFHISKDEF |
| | | NDFSKWMYAFDETPKYKDYLRELFSQKGFFDNPAYKKLFEDSVS |
| | | LDTMYLQTKKNYEEWLKTFVPGQRATDKYSLNNYVKFFDNDLFF |
| | | INISHFIHFLESRNKLQREENGNIIYHSLSNSEYLINEYYFKDKLEKS |
| | | EYKTCGKLYNKLKTIKLEDSLLYEMSMHYLQIDKSIVKNARTSIIN |
| | | LLVQDVKFSIEDANKNHLYDLFIPFNKIDSFVELIKHKEEQEQDKR |
| | | FGGNSFLSKLSSYIEKVRNNKDIKPIFDRFIKKNSLNFDDLNKINNH |
| | | IITNSAKFTKVELSLEEYFIFKDKILIKKENRINIDEIKNLSKYFNKV |
| | | DRHNAFHFNVPNECYDIFLNKIESTYVKDEVRPVAPKQYPDLTKQ |
| | | QRSVCSTFLDAIHNDFFNRSDREKMREEAENKYFQKIILNRMN |
| 19 | Cas13i1 | MSIKITVGSSNDGFTRHETKWHDSDWDENDDSKVPRCIDIGGSRIG |
| | | ALTEHLEIVVPKDMGTKKKKDEVKDFTLYLNYAVSNLREITGIEG |
| | | DEESKIKERLENLSDEKKQRLADFLWAFRFDEPQRDFAARGNDQK |
| | | QFDHDYKRLAPLVATKIFELRNYFAHLDRRGNEALVCDRELYVLL |
| | | EGILRPLAEKECMGPGCQTSKLYKMHLLNLRGPRKPPQPMESRKY |
| | | DLTRRGVMFLVCMALYKDDAEEFLSCFADMRVPNRRRDSLDGLS |
| | | ETDIGKLGRKKPSKRAFLKAFTFFSYRRGRVSLDGEDPDFLNFANII |
| | | GYLNKVPGASFDYLALEDERKLLADLASRSTESEENKLFKYDLSK |
| | | QVRAKDRFVSLAAAWCERFDVLPCIHFKRLDITPSLGRHRYLFGK |
| | | ENDNTVHLDRHYIIENGAIRFEWRPTTHYGDIKIGYLRSCIGEAEFR |
| | | RLLFCALRKPAETNAMLDAYFTAYHKILELMLNAPALDSFSIFSND |
| | | ELVEAVQTVTGLTADELADIPEERLRQFFPKNLWRFFFAWEASQD |
| | | DDDLRAAVAAKIDGRIKWCSDFLVRVENFRKWKAENFWKPNDQ |
| | | RGPQGACPKSKLVNPPRNCRTSDASCVARVIAYLNYHLAPERKFR |
| | | QLSLGKQHSHPGRNDNDTHNYEYQLVQSAIGKYALDQTSAVGGT |
| | | KNGRVIKGTVAHLRPELELHLAQLRDKVRALARENLQPMEDSLR |
| | | AAKRKNLLSGFACLDADKALKSARNSRKLIDLAEAAAELLKEELL |
| | | KEAESLLSVGGDALKVLCRKFGVRTGLPLDKDALVKTILGIDMDK |
| | | WTHAFDYGHGHPYVDRKLTEGDHVAAQIPLPNGMAERVMRSQS |
| | | RVFDGLIGESGIDWAKAFASLDEKNISLLGFYDVSPLVGYIKTHSD |
| | | HTEEEPDSSAPGINTWADADSRTIKPDFSRGGINKAIRAIKTAHNQ |
| | | DRLLLKFAIQHWEEFKKTRAYDERKTAYEKALSVRDYFNGTFTY |
| | | ALKGGVGLRLSRNDVFSPTFTHVVENAAAIVDLLKHENPDVKEVS |
| | | FYDVAQLFARKQRESRKLRMELLPLIEQFGAKSPIPQEEYTRINAE |
| | | GGKNDERKKKIRAMEYSYYSKAMPTLTEAEYNLVADVRNAVMH |
| | | STLLVTDDDAYRAAKAVLGRLVSAR |
| 20 | Cas13j1 | MDTPSKESSGKIIEKLDELKKATQESIEQRKKFDYRIGKGLADKLG |
| | | NEKDSYFEKYLRKTVGENIPPKILVKPKGGWKTETKGKGKDWGD |
| | | KITFAAMPQHHYNTLLAMAFTKAHKVYSDIRGKVEIDAAQYETK |
| | | AKLIEVEYGKDETRGLSGLEYLMMSKHLFSGKNSNIKLAVKKGET |
| | | EILKEYALNEKLIYTVLDELRNFHSHIFHEPGPVSFKNLYGDEYKP |
| | | EKKLTEEEWAIARDWFVNRFNDAKEHKLKTLAKVLEREGTTEEK |
| | | EDAEKVIKTISGYSFEYNNCISREALLFIACMFLRKSDAAYFTKKW |
| | | TGMKKAEGVFKSTQSFFTDNALKESKSILTLNADLYKYRQILGVL |
| | | STMPAMKTDSLKPFYDFIKINNDSYSEKAEKARSKEEKEKIQAFIIP |
| | | QRKSSNYTYWFMKYLNDNKLLDGFRIAYYKTPEDRFMYLIHNGL |
| | | ISQDDLENIEDFKTPDEKLKYLREKGFNLKLKMKQAVGDEKKSLT |
| | | EIYKETQRNFVFKVPTIENDNFCVKKLNVFFQTDIEFNGQKISVQLS |
| | | VSPDFLMKWVFVLLITGEDSIKNAITEKKEKIKDILKKYAEEYYNR |
| | | CITSNFNEPLMGLEASKVFPSSLTSTVEIDEKIDKDKILMRISEKYN |
| | | ELTKFDEENKSRKAPWRFASKRKIDIILDYVHLVYSDRAFDEKKS |
| | | VDAMRHEALNDMEYMDTFEYLRYYGRYRETEEFKKIFFEDKKLY |
| | | FSPILKAMKQLDSLEGVFNFAITGFLNYLKGIQSKVTDENTNKYGK |
| | | VFKVTGKSLTSKIGHHSEMFSVNHCVPQELIKLNDIKGYMKWKHE |
| | | TKDKLWISDFAFIRNVLESRGGFSNTDYLMKEVMPLITFEKNEKGS |
| | | IKGNTQMFVALSRNKTNELMLWEIGKYYWKEATGSEFSRLFKGL |
| | | EKNTGNKITKAYRFTNPYYTIYQEDLDIKIQRKDKKGKVIVNSPVY |
| | | TIKIKPKKFDDEYQYYEQEHIVDYIENYEPKKGIDGHWHFEELNK |
| | | KIKDELARYLDDIYLLMTVEKHIVQKDFDKYASLLKEKDSKLPPY |
| | | YIGFKRNDIALNKVIFDALKHKGSFSADDLNIYRINVLHQILQPKRE |
| | | KYIIIRKSLIEYCAENKLLKTM |
| 21 | Cas13k1 | MKKELNREDVFTGGKTPELTIYYNIAYFRMAGLINALCGNKLERD |
| | | KDALEQFMKAFINGKQKLTDMQFVKLCDYLWKGYKYDKNRSEY |
| | | SLTENDKTMVLKMVAKLQDIRNFQSHIWHDNKVLVFDADLVQFI |
| | | KNKHLEAIDAQRQRFSKETEVYEKENRELKKKKNKELLFDIHDGL |
| | | GYITGEGRNFFLSFFLTRGEMTRFLKQRKGCKRDDTPEYKIKHLV |
| | | YRHFTHRDGATRTHYGYEDNMLDQLPDKQEFLTTRHTYRLINYL |
| | | NDIPEEVTNPELFPLFNTISNGILIKESVGTYIAFINKMPLLSDFEFSE |
| | | VWGKDGKPYTNLVEFYHKTQPGFKFRTDLNSFHKIILNLIRDENY |
| | | TGFFTTQLNLFMAHREELILVISKPLITPNDLLLIEEHYRYKLKAND |
| | | FVREKLGEWKEKIEKGKWEKANEQKDKLINLLGGVIEITFYDFYW |
| | | QKDEKPRNENRFMEFAIRFMADFNLLPDCEWEVEPLQIENDLIAN |
| | | RTATPDLKKSGSQFMNQLTDNYRLKFNDGQIAFRYQNQVFIMGH |
| | | KAVKNLLIACFDGKEKMLNGFMPALLADLKKITNEHILKNHQAL |
| | | NTLSLLNNDSIPSYISRQWGEAEPITDMKKKAIARMDYITQQFEALI |
| | | ENHHYLNRADKNRQIMRCYKFFEWQYPQNSQFKFLRRNEYHRM |
| | | SIYHYCLDKEQHKYDKKGHNYLYNRLIKESHNESGNIEQHLPYQI |
| | | RTMLNDAKDFNDYFLRILNATHKILTDWKNQLKQGREPNNYYLS |
| | | RLGFTGGLTQKVVHTRLLPFSIHPGIPVSFFYRTEMNQNPSFNLSA |
| | | KVWNSESPFRVGLKESNYQYAKYLGLFNEIKTQRKIIGKMNQLIA |
| | | EDALLWQIAKKYLDKCHPKFSEIINQQVNGKQPLNVKAIHDTELTI |
| | | DFPVDKNLYQVVVKMKQLDDFMFIDSKPVLEKTIRYMIKNSLRD |
| | | KEYQQRVFIETNPGVFKIPYNEVVEAMRTINHQALTWARLLLDWE |
| | | SKTVKTIPEADKKAKLQEAYNKRQPAFFNFNDICRYADDGKQLK |
| | | DLRNKTFHTEVPETWTYDEMRENKTIKMVLGINETTFLKKDYAIE |
| | | NARTIE |
| 22 | Cas13d_896 | MKKNSNDKTNAKRMGIKSFIKNGDERFITTSIKNEFPVELKLDVIK |
| | aa | KTCEPAHEPVSFDYDPKKIDFEKPVLKEKLTSGQSGQKLSTRLFIQ |
| | | KDRDICGIRRKYLEKIFNSNFIEEKKDSNLPMQIVAKVLSTEKVFSN |
| | | ALNKIISQFLSMPRGGVTDNHGEYEIIGNIINHKSLQELNKEKKTKR |
| | | IKKYLQSVIKNQSYLYNKQFLLSLDESKGSRNDIDENELYDYIRFL |
| | | AILRNGIAHVFYEKNEPETAKESLFRLVDFIKNDKKLEGAFAKIKIQ |
| | | VNTLYKCRKEEYIKKSGKNFEIIRKIYQNDKPDEKVKDWIRYDFD |
| | | KSYKYIGLSVAKLGNYTSWAKDIDNLRDKSNPDSGYAGIMHRLN |
| | | EFSVYLKVKALSTEEKDKYLKNLISKENCEEKDKYYKNIAQFFCSS |
| | | DLKFANVLQMVKEIKKNKGCTSEDKNCKLCVDERKENDLSVIVY |
| | | FISCFLDNKDQNIFLSDLINRFGALSDLLRIQNKILGAGNKYNENYS |
| | | FLKNERYVTEIKMELETIFALVKVSYKKEDKAFNRLLEDGLVMFG |
| | | FSKDEAGMKVAGLKEIKEKKEGHYKNKSRSFLINSIVNSRKFAYL |
| | | AKSIDPQKVPAIIKNEHIVRYILGRINKTNPGQIGRYWRYIMSQNH |
| | | AGTDKVDDLTNEIIKINIKNILNDAGGWQKSKLNDNNNKKKLKYQ |
| | | QLIGLYLTVAYIFVKNMLECNARYFSAFAQIEKDYLIYTNSDEFYY |
| | | IDKNKKNLVTERYLKLVKDIIEKNKNTVRKDKIFRKKRQRKHLAD |
| | | ISKSIIEFEKLPCCIFTLLRNITEHLNVASNIDIIEGYGKRAGKYHKN |
| | | APASYFIFYHYIIQKILADKICTRNLLNIINTYGEPSISFIKIIYVPFAY |
| | | NLPRYLNLTDARIFCNMDDK |
| 23 | Cas13d_911 | MQQNSTDNKNKIKKSAAKAVGVKSLARLSDGSTVFSSFGKGAAA |
| | aa | ELESLITGGEIRKLSDKAILEITDDTQNKNAYNVKSYRIPNLTARTD |
| | | KLSDKSGMDDLGFKRELELEVFGQSFDDSIHIQIAHAVFDIQKSLA |
| | | AVIPNVLYTLNNLDRSYSTDDTAGKKDIIGNTLNYQHSYDNFDKE |
| | | KLGEFTEYYNAAKDRFSYFPDILCVLEKVNGKDRYQPKSEKDAFN |
| | | VLSSVNMLRNSLFHFAQKSNDGKARIAVFKNQFDSDFSHITSTVN |
| | | KIYSAKIAGVNENFLNNEGNNLYIILNATNWDIKKIVPQLYRFSVL |
| | | KSDKNMGFNMRRLREFAVESKSIDLSRLNDKFLTNNRKKLYKVID |
| | | FIIYYHLNKVRKDTFKDDFVAALRASQSEEEKEKLYAQYSERLFA |
| | | DEGLKSAIKKAVDMISDTKSSIFKKKTPLDKALIENIKVNSDASDF |
| | | CKLIYVFTRFLDGKEINILLNSLIKKFQDIHSFNTTVKKLSENNLIIN |
| | | ADYVDDYSLFEQSGTVAKELMLIKSISKMDFGLDNINLSFMYDDA |
| | | LRTLGVSDENLPEVKREYFGKTKNLSAYIRNNVLENRRFKYVIKYI |
| | | HPSDVQKIACNKAIAGFVLNRMPDTQIKRYYDSLINKGATDMKA |
| | | QAKALLDCITGISFD AIKDDKHLHKSKEKSPQRSADRERKKAMLT |
| | | LYYTIVYIFVKQMLHINSLYTIGFFYLERDQRFIYSRAKKENKNSSK |
| | | NSYLNDFRSVTAYFIPSEIMKRIEKNENKGFLEDFEALWNSCGKTS |
| | | RLRKEDVLLYARYISPDHALKNYKMILNSYRNKIAHINVIMSAGK |
| | | YTGGIKRMDSYFAVFQHLVQCDILSNPNNANCFKSESLKPLLLDM |
| | | RFDGTDEKLYSKRLTRALNIPFGYNVPRYKNLTFEKIYLKSSINE |
| 24 | Cas13d_942 | MAKKKRITAKERKQNHRESLMKKADSNAEKEKAKKPVVENKPD |
| | aa | TAISKDNTPKPNKEIKKSKAKLAGVKWVIKANDDVAYISSFGKGN |
| | | NSVLEKRIMGDVSSNVNKDSHMYVNPKYTKKNYEIKNGFSSGSSL |
| | | VTYPNKPDKNSGMDALCLKPYFEKDFFGHIFTDNMHIQAIYNIFDI |
| | | EKILAKHITNIIYTVNSFDRNYNQSGNDTIGFDINYRVPYSEYGGG |
| | | KDSNGEPKNQSKWKKRKNFIKFYNKSKPHLGYYENIFYDHGEPIS |
| | | EEKFYNYLNILNFIRNNTFHYKDDDIELYSENYSEEFVFINCLNKFV |
| | | KNKFKNVNKNFISNEKNNLYIILNAYGKDTENVEVVKKYSKELYK |
| | | LSVLKTNKNLGVNVKKLRESAIEYGYCPLPYDKEKEVAKLSSVKH |
| | | KLYKTYDFVITHYLNSNDKILLEIVEVLRLSKNDDEKENVYKKYA |
| | | EKLFKADDVINPIKAISKLFAEKGNKLFKEKIIIKKEYIEDVSIDKNI |
| | | YDFTKVIFFMTCFLDGKEINDLLTNIISKLQVIEDHNNVIKFISHNK |
| | | DAVYKDYSDKYAIFRNAGKIATELEAIKSIARMENKIENAPQEPLL |
| | | NDALLALGVSKTDLENTYNKYFDSKEKTDKQSQKVSTFLMNNVI |
| | | NNNRFKYVIKYINPADINGLAKNRYLVKFVLSKIPEEQIDSYYKLF |
| | | SNEEEPSCEEKIKLLTKKISKLNFQTLFENNKIPNVEKERKKAIITLY |
| | | FTIVYILVKNLVNINGLYTLALYFVERDRYFYKKICGKALRRKVG |
| | | DKYDYLLLPEIFSGSKYREETKNLKLPKEKDRDIMKKYLPNDKDR |
| | | EGYNDFFTAYRNNIVHLNIIAKLSELTKNIDKDINSYFDIYHYCTQR |
| | | VMFDYCKMNNNVVLAKMKDLAHIKSDCDEFSSKHTYPFSSAVLR |
| | | FMNLPFAYNVPRFKNLSYKKFFDKQWLNH |
| 25 | Cas13d_957 | MAKKKRITAKERKQNHRESLMKKADSNAEKEKAKKPVVENKPD |
| | aa | TAISKDNTPKPNKEIKKSKAKLAGVKWVIKANDDVAYISSFGKGN |
| | | NSVLEKRIIGDVSSDVNKDSHMYVNPKYTKKNYEIKNGFSSGSSL |
| | | TTHPNKPDKNSGMDALCLKIYFEKEIFKDKFNDNMHIQTIYNIFDI |
| | | EKTLAKHITNIIYAVNSLDRSYIQSGNDTIGFGLNYRIPYAKYGRG |
| | | KDSNGKPNNSNLKKRESFIKFYNNAKDRFGYFESVFYQNGKPISR |
| | | EKLYIYLNILNFVRNSTFHYNNTSTYLYRKEYKYTDKDNCSVKEF |
| | | EFVSYLNEFVKNKFKNVNKNFISNEKNNLYIILNAYGEDIEDVEVV |
| | | KKYSKELYKLSVLKTNKNLGVNVKKLRESAIEYGYCPLPYDKEK |
| | | EVAKLSSIKHKLYKTYDFVITHYLNSNDKLLLEIVEALRLSKNDDE |
| | | KENVYKIYAEKIFKAEYVINPIKTISNLFAEKGDKLFNEKVSISEEY |
| | | VEDIRIDKNIHNFTKVIFFLTCFLDGKEINDLLTNIISKLQVIEDHNN |
| | | VIKAIANNNDAVYKDYSDKYAVFKNSGKIATKLEAIKSIARMENK |
| | | INKAFKEPLLKDAMLALGVSPNDLDEKYEKYFKTDVDADKDHQK |
| | | VSTFLMNNVINNSRFKYVVKYINPADINRLAKNKHLVKFVLDQIP |
| | | HKQIDSYYNSVCTVEEPSYKGKIQLLTKKITGLNFYSLFENCKIPN |
| | | VEKEKKKAVITLYFTIIYILVKNLVNINGLYTLALYFVERDGFFYK |
| | | KICEKKDKKKTNKDVDYLLLPEIFSGSKYREETKNLKLPKEKDREI |
| | | MKKYLPNDEDRKEYNKFFKQYRNNIVHLNIIANLSKLTSTIDKEIN |
| | | SYFEIFHYCAQRVMFDYCKNNNKVVLAKMKDLAHIKSDCDEFSS |
| | | KYTYPYSSAVLRFMNLPFAYNVPRFKNLSYQKFFDKQRLEALEKN |
| | | LNI |
| 26 | Cas13d_968 | MGKKIHARDLREQRKNDRTTKFAEQNKKREAQMAVQKKDAAVS |
| | aa | AKSVSSVSSKKGNVTKSMAKAAGVKSVFAVGKNTVYMTSFGRG |
| | | NDAVLEQKIVDTSHEPLNIDDPAYQLNVVTMNGYSVTGHRGETV |
| | | SAITDNPLRRFNGGKKDKPEQSVPADMLCLKPTLEKKFFGKEFDD |
| | | NIHIQLIYNILDIEKILAVYSTNAVYALNNTIADENNENWDLFANFS |
| | | TDNTYGELINAATYKESTDDVSTDDEKRREAEKKKREAKIAEKIL |
| | | ADYEKFRKNNRLAYFADAFYIEKNKSKSKSQNKAEGIKRGKKEIY |
| | | SILALIAKLRHWCVHSEDGRAEFWLYKLDELEDDFKNVLDVVYN |
| | | RPVEEINDDFVERNKVNIQILHSKCENSDIAELTRSYYEFLITKKYK |
| | | NMGFSIKKLREIILEGTEYNDNKYDTVRNKLYQMVDFILYRGYIN |
| | | ENSERAEALVNALRSTLNEDDKTKLYSSEAAFLKRKYMKIIREVT |
| | | DSLDVKKLKELKKNAFTIPDNELRKCFISYADSVSEFTKLIYLLTRF |
| | | LSGKEINDLVTTLINKFDNIRSFLEIMDELGLERTFTDEYSFFEGSTK |
| | | YLAELIELNSFVKSCSFDMSAKRPMYRDALDILGIESDKSEDDIKR |
| | | MIDNILQVDANGKKLPNKNHGLRNFIASNVVESNRFEYLVRYGNP |
| | | KKIRETAKCKPAVRFVLNEIPDAQIERYYKAYYLDEKSLCLANMQ |
| | | RDKLAGVIADIKFDDFSDAGSYQKANATSTKITSEAEIKRKNQAIIR |
| | | LYLTVMYIMLKNLVNVNARYVIAFHCLERDTKLYAESGLEVGNIE |
| | | KNKTNLTMAVMGVKLENGIIKTEFDKSLAENAANRYLRNARWY |
| | | KLILDNLKMSERAVVNEFRNTVCHLNAIRNININIDGIKEVENYFA |
| | | LYHYLIQKHLENRFADNGGSTGDYIGKLEEHKTYCKDFVKAYCTP |
| | | FGYNLVRYKNLTIDGLFDKNYPGKDDSDKQK |
| 27 | Cas13d_982 | MGKKIHARDLREQRKNDRTTKFAEQNKKREAQMAVQKKDAAVS |
| | aa | AKSVSSVSSKKGNVTKSMAKAAGVKSVFAVGKNTVYMTSFGRG |
| | | NDAVLEQKIVDTSHEPLNIDDPAYQLNVVTMNGYSVTGHRGETV |
| | | SAITDNPLRRFNGGKKDKPEQSVPADMLCLKPTLEKKFFGKEFDD |
| | | NIHIQLIYNILDIEKILAVYSTNAVYALNNTIADENNENWDLFANFS |
| | | TDNTYGELINAATYKESTDDVSTDDEKRREAEKKKREAKIAEKIL |
| | | ADYEKFRKNNRLAYFADAFYIEKNKSKSKSQNKAEGIKRGKKEIY |
| | | SILALIAKLRHWCVHSEDGRAEFWLYKLDELEDDFKNVLDVVYN |
| | | RPVEEINDDFVERNKVNIQILHSKCENSDIAELTRSYYEFLITKKYK |
| | | NMGFSIKKLREIILEGTEYNDNKYDTVRNKLYQMVDFILYRGYIN |
| | | ENSERAEALVNALRSTLNEDDKTKLYSSEAAFLKRKYMKIIREVT |
| | | DSLDVKKLKELKKNAFTIPDNELRKCFISYADSVSEFTKLIYLLTRF |
| | | LSGKEINDLVTTLINKFDNIRSFLEIMDELGLERTFTDEYSFFEGSTK |
| | | YLAELIELNSFVKSCSFDMSAKRPMYRDALDILGIESDKSEDDIKR |
| | | MIDNILQVDANGKKLPNKNHGLRNFIASNVVESNRFEYLVRYGNP |
| | | KKIRETAKCKPAVRFVLNEIPDAQIERYYKAYYLDEKSLCLANMQ |
| | | RDKLAGVIADIKFDDFSDAGSYQKANATSTKITSEAEIKRKNQAIIR |
| | | LYLTVMYIMLKNLVNVNARYVIAFHCLERDTKLYAESGLEVGNIE |
| | | KNKTNLTMAVMGVKLENGIIKTEFDKSLAENAANRYLRNARWY |
| | | KLILDNLKMSERAVVNEFRNTVCHLNAIRNININIDGIKEVENYFA |
| | | LYHYLIQKHLENRFADNGGSTGDYIGKLEEHKTYCKDFVKAYCTP |
| | | FGYNLVRYKNLTIDGLFDKNYPGKDDSDKQK |
| 28 | Cas13d_999 | MGNKQRVSAQKRRENAKLCNQQKARQAESQRDKIKNMNVEKM |
| | aa | KNINTNDIKHTKTTAKKLGLKSTIIADKKIILTSFINEQSSKTANIEK |
| | | VAGFKGDTIDTISYTPRMFRSEINPGEIVISKGDDLSEFANPANFPIG |
| | | RDYVKIRSALEKQYFGKEFPEDNLHVQIAYNVADIKKILSVYINNII |
| | | YMFYNLARSEEYDIFYNSQSENSGRDCDVIGSLYYQASYRNQDAN |
| | | RFEKDGKKKAIDSLLDDTRAYYTYFDGLFSVPKREDDGKIKESEK |
| | | EKAKDQNFDVLRLLSVGRQLTFHSDKSNNEAYLFDLSKLTRAAQ |
| | | DENRRQDIQSLLNILNSTCRSNLEGVNGDFVKHAKNNLYVLNQLY |
| | | PSLKANDLIGEYYNFIVKKENRNIGIRLITVRELIIEHNYTNLKDSK |
| | | YDTYRNKIYTVLNFILFREIQENSIAIKNFREKLRSTEKAEQPALYQ |
| | | AFANKIYPMVQAKFAKAIDLFEEQYKTKFKSEFKGGISIENMQQQ |
| | | NILLQTENIDYFSKYVLFLTKFLDGKEINELLCALINKFDNIADLLDI |
| | | SKQIGTPVVFCADYESLNDAAKIAENIRLIKNIAHLRPAIQEAQSSK |
| | | DNADAAGTPATLLIDAYNMLNTDIQLVYGEAAYEELRKDLFERK |
| | | NGTKYNKKGKKVDVYDHKFRNFLINNVIKSKWFFYIAKYVKPAD |
| | | CAKMMSNKKMIEFALRDLPETQIKRYYYTITGNEALGDAESLKGV |
| | | IIEQLHAFSIKNTLLSIKNMGEGEYKIQQIGSSKEKLKAIVNLYLTV |
| | | AYLLTKSLVKVNIRFSIAFGCLERDLVLQKKSEKKFDAIINEILLED |
| | | DKIRKECDKERAQAKTLPRELAQERFAQIKRRESGCYFKSYHVYD |
| | | YLSKNSNEFKQNHIDFAVTSYRNNVEHLNVVHCMTKYFSEVKDV |
| | | KSYYGVYCYIMQRMLCDELIIKNQDKPDVRQTFEEYNRLLKDHG |
| | | TYSKNLMWLLNFPFAYNLARYKNLSNEDLFNAKNNDQKSK |

TABLE 3

| SEQ | | |
|---|---|---|
| ID | | |
| NO. | crRNA name | Spacer sequence |
| 57 | mCherry- | GTTCATCACGCGCTCCCACTTGAAGCCCTC |
| | spacer-1 | |
| 58 | mCherry- | TGCTTCACGTAGGCCTTGGAGCCGTACATG |
| | spacer-2 | |
| 59 | NT-spacer-1 | CCTCTGAAACGATGGTGCATGGTAGTGACC |
| 60 | ANXA4-spacer-1 | AATTAGGCAGCCCTCATCAGTGCCGGCTCC |
| 61 | ANXA4-spacer-2 | CTTGTAGGCTGTCCTGATCTCCTGGCGCTG |
| 62 | EZH2-spacer_1 | GAGAGCAGCAGCAAACTCCTTTGCTCCCTC |
| 63 | EZH2-spacer_2 | CAGAGGAGCTCGAAGTTTCATCTTTCTTCT |
| 64 | EZH2-spacer_3 | GTATCCTTTGATTCCAGCACATTAATGGTG |
| 65 | NF2-spacer_1 | CTTGTGAACACTGGGGTCGTAGTCACCATA |
| 66 | NF2-spacer_2 | TAGTCACCATACTTGGCCTGGACGGCGTAA |
| 67 | NF2-spacer_3 | CCTTTTTGGAAGCAATTCCTCTTGGGCCAA |
| 68 | HRAS-spacer_1 | CTGTACTGGTGGATGTCCTCAAAAGACTTG |
| 69 | HRAS-spacer_2 | TCCGAGTCCTTCACCCGTTTGATCTGCTCC |
| 70 | HRAS-spacer_3 | TGTTCCCCACCAGCACCATGGGCACGTCAT |
| 71 | NRAS-spacer_1 | TACCAGTGTGTAAAAAGCATCTTCAACACC |
| 72 | NRAS-spacer_2 | CTGTAGAGGTTAATATCCGCAAATGACTTG |
| 73 | NRAS-spacer_3 | CGCTTAATCTGCTCCCTGTAGAGGTTAATA |
| 74 | PPARG-spacer_1 | CATTATGAGACATCCCCACTGCAAGGCATT |
| 75 | PPARG-spacer_2 | CGGCCTGTGGCATCCGCCCAAACCTGATGG |
| 76 | PPARG-spacer_3 | AAACCTGATGGCATTATGAGACATCCCCAC |
| 77 | STAT3-spacer | TAGCATCCATGATCTTATAGCCCATGATGA |
| 78 | EGFR-spacer | GTTTCTGGCAGTTCTCCTCTCCTGCACCCC |
| 79 | KRAS-spacer | GAAAGCCCTCCCCAGTCCTCATGTACTGGT |
| 80 | CXCR4-spacer | ATAAGGCCAACCATGATGTGCTGAAACTGG |

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13-2A-eGFP plasmid, 300 ng pUC19-U6-5′-DR crRNA plasmid or pUC19-U6-3′-DR crRNA plasmid, and 10 ng pCAG-eBFP-EF1a-mCherry plasmid. The control group was only transfected with 600 ng 10 pCAG-Cas13-2A-eGFP plasmid and 10 ng pCAG-eBFP-EF1a-mCherry plasmid. After 48 hours, the cells were digested with 0.25% Trypsin-EDTA (Gibco), and the obtained suspension cells were subjected to fluorescence-activated cell sorting (FACS) (FIG. 11A).

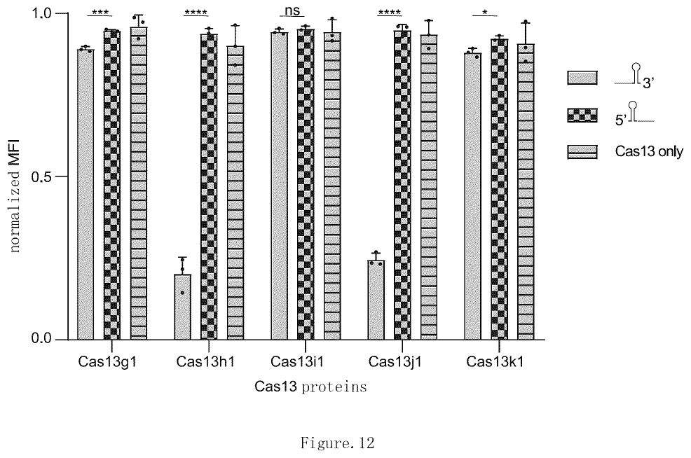

Targeting mCherry mRNA to Identify Novel Cas13 Isoform crRNA Directions mCherry expression was analyzed by FACS 2 days after transfection. Comparing the expression of mCherry corresponding to 5′-DR crRNA and 3′-DR crRNA, it was found that the knockdown efficiency of 3′-DR crRNA corresponding to Cas13g1, Cas13h1, Cas13i1, Cas13j1, and Cas13k1 was higher than that of 5′-DR crRNA. It was proved that Cas13g-k corresponds to the crRNA direction of 3′-DR (FIG. 12). The specific editing result information was shown in Table 4. In FIG. 12, each group had 3 biological replicates, and statistical significance was evaluated using a two-tailed t-test.

TABLE 4

| | 3′-DR | 5′-DR |
|---|---|---|
| Cas13g1 | 0.889667 | 0.947 |
| Cas13h1 | 0.200667 | 0.938 |
| Cas13il | 0.943667 | 0.952333 |
| Cas13j1 | 0.244333 | 0.948333 |
| Cas13kl | 0.878667 | 0.921667 |

### Example 4: Screening Novel Cas13 Proteins With High Editing Efficiency by Targeting Genes in Vivo or In Vitro

### Plasmid Construction

The coding sequence of Cas13 was codon optimized (human) and synthesized. The synthesized Cas13 effector protein sequence was inserted into the XmaI and NheI restriction sites of the pCAG-2A-eGFP vector to construct the pCAG-Cas13-2A-eGFP plasmid. The protein sequences used were shown in Table 2. The synthesized crRNA sequence (U6 promoter sequence+spacer sequence+DR sequence) was inserted between the EcoRI and HindIII restriction sites of the pUC19-U6 vector to construct the pUC19-U6-3′-DR crRNA plasmid. The spacer sequence information was shown in Table 3. The CAG promoter expressed eBFP protein and the EF1a promoter expressed mCherry protein to construct pCAG-eBFP-EF1a-mCherry plasmid (FIG. 11A).

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13-2A-eGFP plasmid, 300 ng pUC19-U6-3′-DR crRNA plasmid, and 10 ng pCAG-eBFP-EF1a-mCherry plasmid. The control group was only transfected with 600 ng pCAG-Cas13-2A-eGFP plasmid and 10 ng pCAG-eBFP-EF1a-mCherry plasmid. 600 ng pCAG-Cas13-2A-eGFP plasmid, 300 ng non-targeting (NT) U6-crRNA plasmid and 10ng pCAG-eBFP-EF1a-mCherry plasmid were co-transfected into HEK293T cells as a non-targeting control group. After 48 hours, the cells were digested with 0.25% Trypsin-EDTA (Gibco), and the obtained suspension cells were subjected to fluorescence-activated cell sorting (FACS) (FIG. 11A).

Knocking Down mCherry mRNA to Screen for Cas13 Proteins With High Editing Efficiency

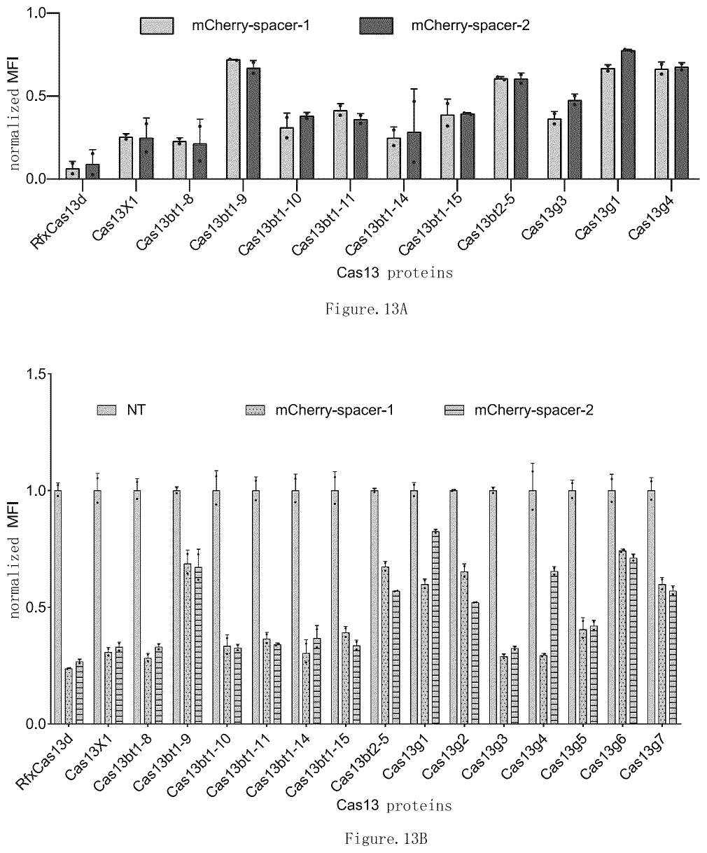

mCherry expression was analyzed by flow cytometry 2 days after transfection. The mean fluorescence intensity (MFI) of EGFP and mCherry double-positive cells in the experimental group was normalized to that of the control group. Preliminary screening of Cas13 proteins with high editing efficiency (FIG. 13A) was performed, wherein the knockdown efficiencies of RfxCas13d, Cas13X1, Cas13bt1-8, Cas13bt1-10, Cas13bt1-11, Cas13bt1-14, Cas13bt1-15, and Cas13g3 were higher than 50%. These proteins were used for the following experiments, the specific editing result information was shown in Table 5A.

mCherry expression was analyzed by flow cytometry 2 days after transfection. The MFI of EGFP and mCherry double-positive cells in the experimental group was normalized to that of the non-target control group. The MFI results (FIG. 13B) and specific editing result information were shown in Table 5B.

TABLE 5A

| | mCherry-spacer-1 | mCherry-spacer-2 |
|---|---|---|
| RfxCas13d | 0.06285 | 0.08915 |
| Cas13X1 | 0.2535 | 0.2475 |
| Cas13bt1-8 | 0.227 | 0.2125 |
| Cas13bt1-9 | 0.718 | 0.6685 |
| Cas13bt1-10 | 0.3095 | 0.3785 |
| Cas13bt1-11 | 0.413 | 0.359 |
| Cas13bt1-14 | 0.2475 | 0.28335 |
| Cas13bt1-15 | 0.387 | 0.3925 |
| Cas13bt2-5 | 0.606 | 0.6025 |
| Cas13g3 | 0.3625 | 0.474 |
| Cas13g1 | 0.6665 | 0.7745 |
| Cas13g4 | 0.6625 | 0.675 |

TABLE 5B

| | mCherry-spacer-1 | mCherry-spacer-2 |
|---|---|---|
| RfxCas13d | 0.2372055 | 0.2672625 |
| Cas13X1 | 0.307399 | 0.3295195 |
| Cas13bt1-8 | 0.2822405 | 0.33014 |
| Cas13bt1-9 | 0.68599 | 0.6714975 |
| Cas13bt1-10 | 0.333556 | 0.326203 |
| Cas13bt1-11 | 0.3649895 | 0.3401845 |
| Cas13bt1-14 | 0.303531 | 0.3666415 |
| Cas13bt1-15 | 0.391273 | 0.3352725 |
| Cas13bt2-5 | 0.6732165 | 0.5691415 |
| Cas13g1 | 0.5981245 | 0.8246755 |
| Cas13g2 | 0.653143 | 0.5203655 |
| Cas13g3 | 0.2890905 | 0.324154 |
| Cas13g4 | 0.294376 | 0.653641 |
| Cas13g5 | 0.4043715 | 0.4201575 |
| Cas13g6 | 0.7418645 | 0.7109505 |
| Cas13g7 | 0.597749 | 0.5702335 |

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13-2A-eGFP plasmid and 300 ng pUC19-U6-3′-DR crRNA plasmid. The control group was only transfected with 600 ng pCAG-Cas13-2A-eGFP plasmid (FIG. 11B). The non-target control group was transfected with 600 ng pCAG-Cas13-2A-eGFP plasmid and 300 ng non-target U6-crRNA plasmid.

RNA Extraction, RNA Quantitative Analysis 48 hours after transfection, cells were lysed using TRIzoI® Reagent (Life Technologie), and then RNA was extracted using PureLink™ RNA Mini Kit (Thermofisher). Finally, RNA quantitative analysis was performed using HiScript® II One Step qRT-PCR SYBR Green Kit (Vazyme). RT-qPCR results were analyzed using the 2^{-ΔΔCT} method. The difference in average CT values between the target gene and the internal reference gene GAPDH in three biological replicates was used to calculate the relative expression of the target gene, and the relative expression of the control group was normalized.
Knocking Down the Gene ANXA4 mRNA In Vivo to Screen for Cas13 Proteins With High Editing Efficiency

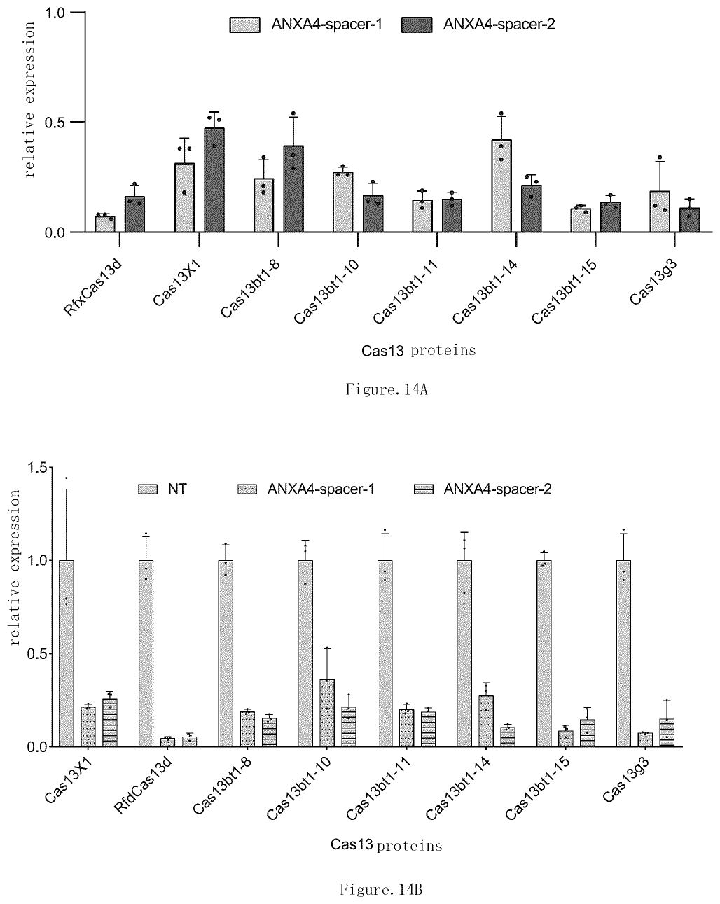

Through the normalized RT-qPCR results targeting ANXA4 mRNA and the control group, it was found that Cas13bt1-11, Cas13bt1-15 and Cas13g3 have higher editing efficiency (FIG. 14A). The specific editing result information was shown in Table 6A. The normalized RT-qPCR results of the experimental group and the non-target control group also showed that Cas13bt1-8, Cas13bt1-11, Cas13bt1-15 and Cas13g3 had higher editing (FIG. 14B). The specific editing result information was shown in Table 6B. It was worth noting that both experiments showed that Cas13g3 has the strongest RNA interference ability and an ultra-small protein molecular weight (767 amino acids). We selected the Cas13g3 protein for further characterization analysis.

TABLE 6A

| | ANXA4-spacer-1 | ANXA4-spacer-2 |
|---|---|---|
| RfxCas13d | 0.073333 | 0.163333 |
| Cas13X1 | 0.313333 | 0.473333 |
| Cas13bt1-8 | 0.243333 | 0.393333 |
| Cas13bt1-10 | 0.273333 | 0.166667 |
| Cas13bt1-11 | 0.146667 | 0.15 |
| Cas13bt1-14 | 0.42 | 0.213333 |
| Cas13bt1-15 | 0.106667 | 0.136667 |
| Cas13g3 | 0.186667 | 0.11 |

TABLE 6B

| | ANXA4-spacer-1 | ANXA4-spacer-2 |
|---|---|---|
| RfxCas13d | 0.215066 | 0.258412 |
| Cas13X1 | 0.046716667 | 0.054699333 |
| Cas13bt1-8 | 0.188513333 | 0.153782667 |
| Cas13bt1-10 | 0.363544667 | 0.214909 |
| Cas13bt1-11 | 0.200584333 | 0.188127667 |
| Cas13bt1-14 | 0.275353333 | 0.105536 |
| Cas13bt1-15 | 0.086085667 | 0.146604667 |
| Cas13g3 | 0.076056 | 0.150550667 |

### Knockdown of Genes In Vivo to Screen for Cas13 Proteins With High Editing Efficiency

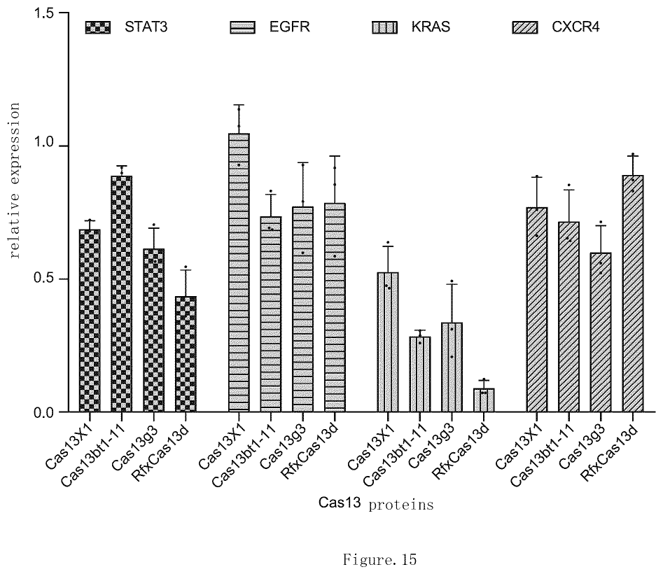

By targeting 4 in vivo genes, it was found that the editing efficiency of RfxCas13d at STAT3 and KRAS sites was higher than that of Cas13g3, while the editing efficiency at EGFR and CXCR4 was lower. Overall, the editing efficiency of Cas13g3 was slightly lower than that of RfxCas13d, but exceeded that of Cas13X1 (FIG. 15), the editing results were shown in Table 7.

TABLE 7

| | STAT3- | EGFR- | KRAS- | CXCR4- |
|---|---|---|---|---|
| | spacer | spacer | spacer | spacer |
| Cas13X1 | 0.687478 | 1.046535 | 0.525891 | 0.769662 |
| Cas13bt1-11 | 0.886987 | 0.735842 | 0.284075 | 0.71624 |
| Cas13g3 | 0.613047 | 0.772218 | 0.336777 | 0.598634 |
| RfxCas13d | 0.435223 | 0.785867 | 0.08919 | 0.890071 |

### Example 5: Verification of Cas13d Homolog Editing Efficiency By Targeting Genes In Vivo

### Plasmid Construction

The coding sequence of the Cas13d homolog was codon optimized (human) and synthesized. The synthesized Cas13 effector protein sequence was inserted into the XmaI and NheI restriction sites of the pCAG-2A-eGFP vector to construct the pCAG-Cas13d-2A-eGFP plasmid. The protein sequences used were shown in Table 2. The synthesized crRNA sequence (U6 promoter sequence+DR sequence+spacer sequence) was inserted between the EcoRI and HindIII restriction sites of the pUC19-U6 vector to construct the pUC19-U6-5′-DR crRNA plasmid. The spacer sequence information was shown in Table 3.

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13d-2A-eGFP plasmid and 300 ng pUC19-U6-5′-DR crRNA plasmid. The control group was only transfected with 600 ng pCAG-Cas13d-2A-eGFP plasmid (FIG. 11B).

### RNA Extraction, RNA Quantitative Analysis

48 hours after transfection, cells were lysed using TRIzol® reagent (Life Technologie), and then RNA was extracted using PureLink™ RNA Mini Kit (Thermofisher). Finally, RNA quantitative analysis was performed using HiScript® II One Step qRT-PCR SYBR Green kit (Vazyme). RT-qPCR results were analyzed using the 2^{-ΔΔCT} method. The difference in average CT values between the target gene and the internal reference gene GAPDH in three biological replicates was used to calculate the relative expression of the target gene, and the relative expression of the control group was normalized.

Knocking Down the Gene ANXA4 mRNA In Vivo to Screen for Cas13 Proteins With High Editing Efficiency

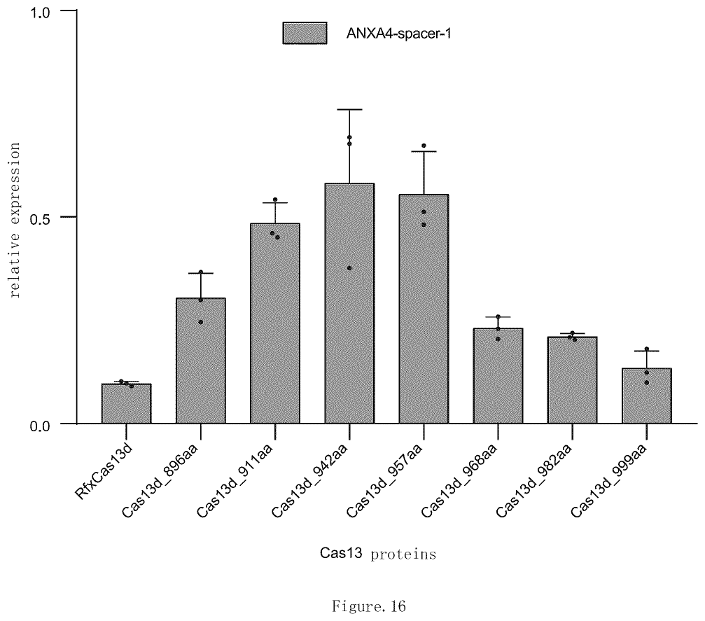

Through the RT-qPCR results targeting ANXA4 mRNA, it was found that Cas13d_968aa, Cas13d_982aa and Cas13d_999aa have high editing efficiency (FIG. 16). The specific editing result information was shown in Table 8.

TABLE 8

| | ANXA4-spacer-1 |
|---|---|
| RfxCas13d | 0.096407 |
| Cas13d_896aa | 0.303705 |
| Cas13d_911aa | 0.484585 |
| Cas13d_942aa | 0.582031 |
| Cas13d_957aa | 0.555765 |
| Cas13d_968aa | 0.230899 |
| Cas13d_982aa | 0.210199 |
| Cas13d_999aa | 0.13415 |

### Example 6: Using RNA-Seq to Explore the Off-Target Activity of Cas13

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13-2A-eGFP plasmid and 300 ng pUC19-U6-3′-DR crRNA plasmid, with crRNA targeting ANXA4 mRNA. The control group was only transfected with 600 ng pCAG-Cas13-2A-eGFP plasmid.

### RNA-Seq

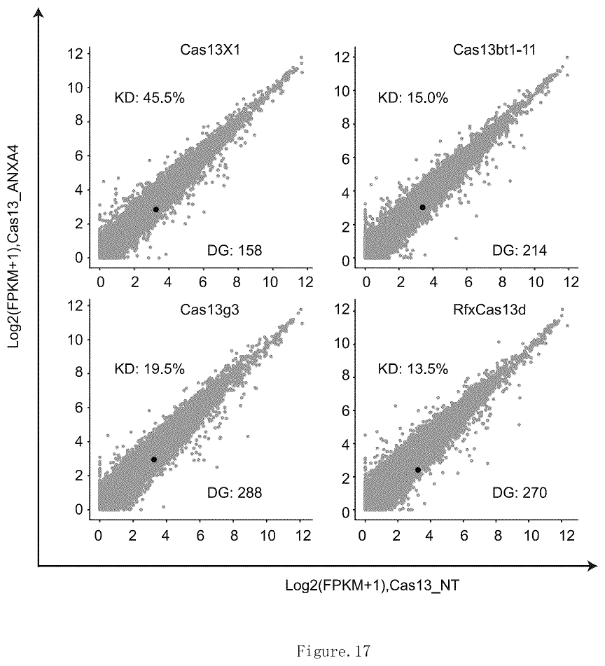

Two days after transfection, cells were lysed using TRIzol® reagent (Life Technologie), and then RNA was extracted using PureLink™ RNA Mini Kit (Thermofisher). Total RNA was extracted and RNA-seq sequenced using the novaseq 6000 platform. The obtained sequencing results were compared to the human genome through STAR software, and then gene expression was calculated through StringTie software. Compared with the control group, it was found that the off-target sites corresponding to Cas13X1, Cas13g3 and RfxCas13d were 102, 133 and 323 respectively. It showed that the specificity of Cas13g3 was comparable to Cas13X1, while its off-target effect was lower than that of RfxCas13d (FIG. 17). In FIG. 17, KD represented the knockdown efficiency, and DG represented the number of down-regulated genes.

### Example 7: Exploring the Trans-Cleavage Activity of Cas13

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection.

### Cell Viability Analysis

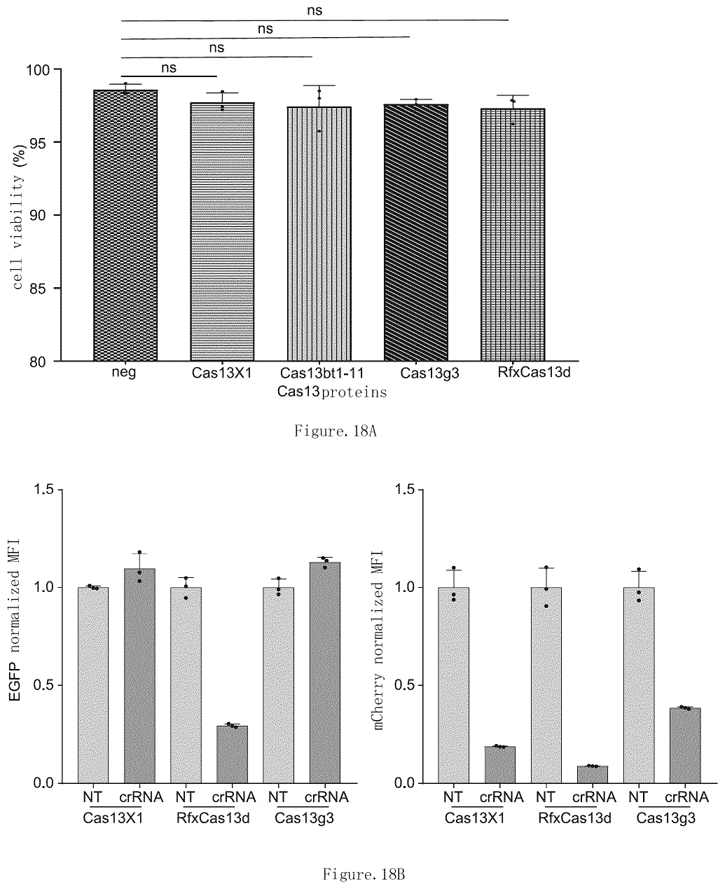

Each well of the cell viability experimental group was transfected with 600 ng pCAG-Cas13-2A-eGFP plasmid and 300 ng pUC19-U6-3′-DR crRNA plasmid, with crRNA targeting ANXA4 mRNA. The control group was not transfected with any plasmid. Calcein-AM/PI double strain kit (Solarbio) was used to measure fluorescent cell viability of HEK293T 2 days after transfection. Total cell quantification was performed at 490 nm excitation and 515 nm emission, and dead cell quantification was performed at 535 nm excitation and 617 nm emission. Measurements were made using flow cytometry. By identifying the viability of transfected cells, it was found that transfection with Cas13 protein did not affect cell viability (FIG. 18A). The specific cell viability information is shown in Table 9A. In FIG. 18, there were 3 biological replicates for each group, and statistical significance was evaluated using a two-tailed t-test.

TABLE 9A

| Neg | Cas13X1 | Cas13bt1-11 | Cas13g3 | RfxCas13d |
|---|---|---|---|---|
| 98.56 | 97.68667 | 97.40333 | 97.57333 | 97.28 |

### Trans-Cleaving Activity

Each well of the trans-cleaving experimental group was transfected with 600 ng pCAG-Cas13-2A-eGFP plasmid, 10 ng pCAG-eBFP-EF1a-mCherry plasmid and 300 ng pUC19-U6-3′-DR crRNA plasmid, in which crRNA targeted mCherry mRNA. The trans-cleaving control group was transfected with non-targeting U6-crRNA plasmid. The cis-cleaving ability of Cas13 protein was calculated by measuring the fluorescence intensity of mCherry, and the trans-cleaving ability was calculated by measuring the fluorescence intensity of EGFP. Consistent with previous results, Cas13X1, RfxCas13d and Cas13g3 could significantly knock down mCherry mRNA (FIG. 18B). Meanwhile, no trans-cleaving activity was detected for Cas13X.1 and Cas13g3, while RfxCas13d showed significant trans-cleaving activity against EGFP transcript (FIG. 18B). The specific MFI result information was seen in Table 9B.

TABLE 9B

| | GFP MFI | mCherry MFI |
|---|---|---|
| Cas13X1 | 1.096344 | 0.188002667 |
| RfdCas13d | 0.294856 | 0.088376333 |
| Cas13g3 | 1.130295 | 0.385392333 |

### Example 8: Characterization of Cas13g3 System With High Editing Efficiency

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection.

### Research on the Optimal Spacer Length of Cas13g3 System

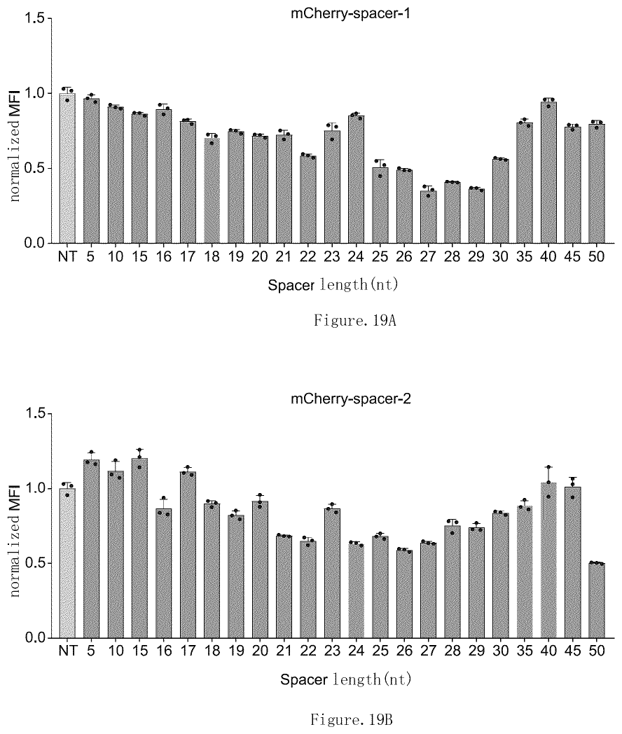

In order to explore the optimal spacer length of the Cas13g3 system, we constructed U6-crRNA plasmids targeting mCherry mRNA with spacer lengths ranging from 5 to 50 nt. When the spacer length is between 15-30 nt, a crRNA was designed every 1 nt; when the spacer length is 5-15 nt and 30-50 nt, a crRNA was designed every 5 nt. 600 ng pCAG-Cas13-2A-eGFP plasmid, 10 ng pCAG-eBFP-EF1a-mCherry plasmid and 300 ng pUC19-U6-3′-DR crRNA plasmid were co-transfected into HEK293T cells as the experimental group. 600 ng pCAG-Cas13-2A-eGFP plasmid, 300 ng non-targeting U6-crRNA plasmid and 10 ng pCAG-eBFP-EF1a-mCherry plasmid were co-transfected into HEK293T cells as a control group. The MFI of EGFP and mCherry double-positive cells in the experimental group was normalized with the negative control. The normalized results can reflect the impact of different spacer lengths on RNA interference. The results showed that when using a spacer with a length of 27 nt, Cas13g3 had the highest average knockout efficiency at the two target sites (FIG. 19). The specific editing result information was seen in Table 10.

TABLE 10

| mCherry-spacer- | normalized | mCherry-spacer- | normalized |
|---|---|---|---|
| 1 (nt) | MFI | 2(nt) | MFI |
| 5 nt | 0.966452 | 5 nt | 1.192277 |
| 10 nt | 0.908946 | 10 nt | 1.116929667 |
| 15 nt | 0.862876333 | 15 nt | 1.202048333 |
| 16 nt | 0.894542333 | 16 nt | 0.864939333 |
| 17 nt | 0.813911333 | 17 nt | 1.111863333 |
| 18 nt | 0.702482667 | 18 nt | 0.896605333 |
| 19 nt | 0.745549 | 19 nt | 0.820027667 |
| 20 nt | 0.717067333 | 20 nt | 0.912239333 |
| 21 nt | 0.724233 | 21 nt | 0.68218 |
| 22 nt | 0.583201 | 22 nt | 0.646931 |
| 23 nt | 0.752605667 | 23 nt | 0.864504667 |
| 24 nt | 0.850934 | 24 nt | 0.630356 |
| 25 nt | 0.506514333 | 25 nt | 0.678995333 |
| 26 nt | 0.490626667 | 26 nt | 0.586783667 |
| 27 nt | 0.35021 | 27 nt | 0.636363667 |
| 28 nt | 0.407426333 | 28 nt | 0.750615333 |
| 29 nt | 0.363202 | 29 nt | 0.738346667 |
| 30 nt | 0.562174333 | 30 nt | 0.835335667 |
| 35 nt | 0.805189667 | 35 nt | 0.88488 |
| 40 nt | 0.943326667 | 40 nt | 1.042414667 |
| 45 nt | 0.777468333 | 45 nt | 1.010639667 |
| 50 nt | 0.795888667 | 50 nt | 0.500325667 |

### Cas 13g3 System PFS Preference Analysis

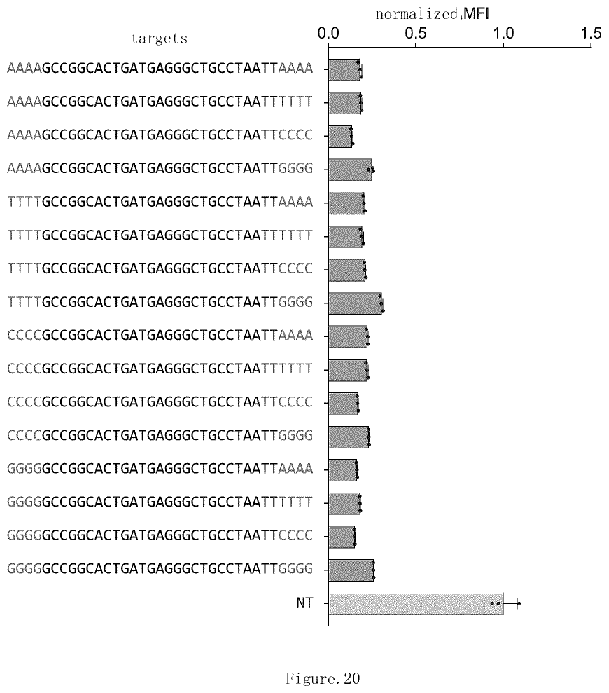

To analyze the PFS preference of the Cas13g3 system, targets of 16 PFS combinations were cloned upstream of the mCherry gene of the EF-1a-mCherry plasmid. The MFI of EGFP and mCherry double-positive cells in the experimental group was normalized with the MFI of the negative control. The experimental group was HEK293T cells transfected with 600 ng pCAG-Cas13-2A-eGFP plasmid, 10 ng pCAG-eBFP-EF1a-mCherry plasmid and 300 ng pUC19-U6-3′-DR crRNA plasmid; the negative control was HEK293T cells transfected with 600 ng pCAG-Cas13-2A-eGFP plasmid, 300 ng non-targeting U6-crRNA plasmid and 10 ng pCAG-eBFP-EF1a-mCherry plasmid. The results showed that Cas13g3 protein demonstrated stable and efficient interference effects in these 16 PFS sequences (FIG. 20). The specific editing result information was shown in Table 11 (the spacer sequence was: SEQ ID NO. 81: GCCGGCACTGATGAGGGCTGCCTAATT).

TABLE 11

| PFS combination | normalized MFI |
|---|---|
| AAAA-spacer-AAAA | 0.18097067 |
| AAAA-spacer-TTTT | 0.18666533 |
| AAAA-spacer-CCCC | 0.134758 |
| AAAA-spacer-GGGG | 0.24784567 |
| TTTT-spacer-AAAA | 0.20475733 |
| TTTT-spacer-TTTT | 0.19346833 |
| TTTT-spacer-CCCC | 0.21004867 |
| TTTT-spacer-GGGG | 0.303835 |
| CCCC-spacer-AAAA | 0.22320233 |
| CCCC-spacer-TTTT | 0.22158933 |
| CCCC-spacer-CCCC | 0.16811967 |
| CCCC-spacer-GGGG | 0.23202133 |
| GGGG-spacer-AAAA | 0.16308 |
| GGGG-spacer-TTTT | 0.18122267 |
| GGGG-spacer-CCCC | 0.15048133 |
| GGGG-spacer-GGGG | 0.258429 |

### Effect of NLS on the Interference Activity of Cas13g3 System

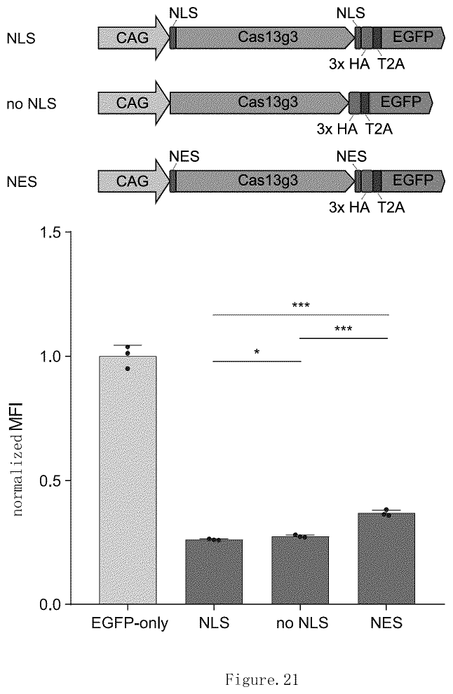

In order to identify the effects of NLS and NES on the interference activity of the Cas13g3 system, we constructed three Cas13 protein expression plasmids fused with NLS, fused with NES, and without fused NLS and NES respectively (FIG. 21). 600 ng pCAG-Cas13-2A-eGFP plasmid, 10 ng pCAG-eBFP-EF1a-mCherry plasmid and 300 ng pUC19-U6-3′-DR crRNA plasmid were co-transfected into HEK293T cells as the experimental group. The control group was only transfected with pCAG-eGFP plasmid. The MFI of EGFP and mCherry double-positive cells was analyzed and calculated. The normalized results compared with the control group showed that the fusion of NLS sequences can improve the knockout efficiency of Cas13g3 compared to the fusion of NES or the absence of any localization signal (FIG. 21). The specific editing result information was seen in Table 12.

TABLE 12

| | normalized MFI |
|---|---|
| NLS | 0.260501 |
| noNLS | 0.274187 |
| NES | 0.367439 |

Therefore, the optimal Cas13g3 editor composition is a crRNA plasmid with a spacer length of 27 nt and a Cas13g3 protein plasmid fused with the NLS sequence.

### Example 9: Optimal Cas13g3 Editor Interference Capability Verification

### Cell Culture, Transfection

HEK293T cells were cultured in DMEM (Gibco) medium containing 10% FBS (Gibco) and 1% Penicillin-Streptomycin (Gibco). The cells were digested with 0.25% Trypsin-EDTA (Gibco), transferred to a 24-well plate and cultured for 12 hours. When the cell density reached 90%, LIPOFECTAMINE 3000 Reagent (Invitrogen) was used for transfection. Each well of the experimental group was transfected with 600 ng pCAG-Cas13-2A-eGFP plasmid and 300 ng pUC19-U6-3′-DR crRNA plasmid. The non-target control group was transfected with 600 ng pCAG-Cas13-2A-eGFP plasmid and 300 ng non-target U6-crRNA plasmid.

### RNA Extraction, RNA Quantitative Analysis

48 hours after transfection, cells were lysed using TRIzol® Reagent (Life Technologie), and then RNA was extracted using PureLink™ RNA Mini Kit (Thermofisher). Finally, RNA quantitative analysis was performed using HiScript® II One Step qRT-PCR SYBR Green Kit(Vazyme). RT-qPCR results were analyzed using the 2^{-ΔΔCT} method. The difference in average CT values between the target gene and the internal reference gene GAPDH in three biological replicates was used to calculate the relative expression of the target gene, and the relative expression of the control group was normalized.

### Comparison of RNA Interference Capabilities Between the Cas13g3 System and Other Cas13 Systems

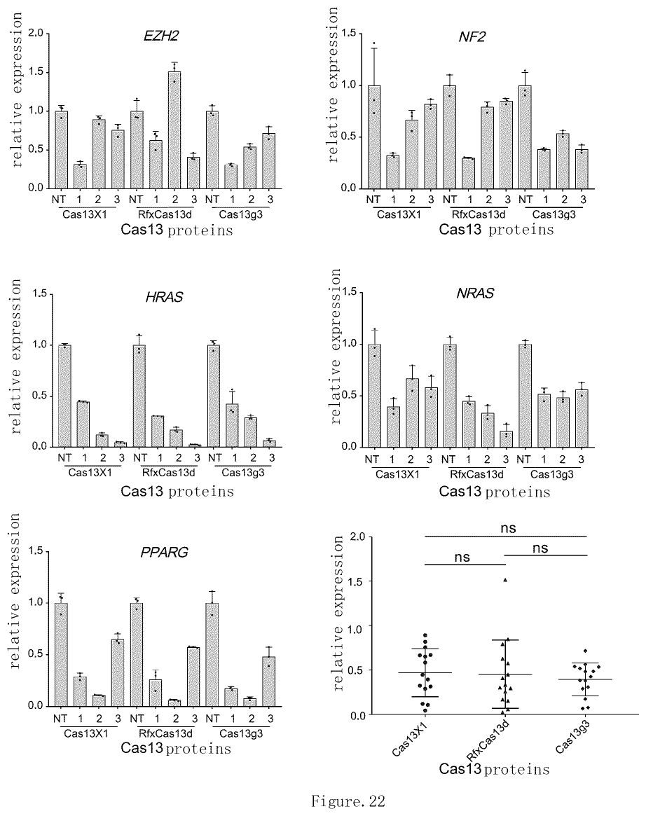

In order to systematically compare the RNA interference activity of Cas13g3 protein with the currently most commonly used and highest cleavage efficiency Cas13 proteins Cas13X1 and RfxCas13d at the same target site and study the knockout efficiency of Cas13g3 protein on a larger scale, we designed a total of 15 crRNAs plasmids targeting 5 endogenous genes. By targeting 5 in vivo genes EZH2, NF2, HRAS, NRAS and PPARG, Cas13X1, RfxCas13d and Cas13g3 can exhibit powerful RNA interference activity on EZH2, NF2, HRAS, NRAS and PPARG genes, and their average knockdown efficiencies are respectively 52.84%, 54.58% and 60.37% (FIG. 22). The specific editing results were shown in Table 13. Paired-t-test statistical analysis of the interference results showed that Cas13g3 has efficient RNA knockdown activity, which is comparable to Cas13X1 and RfxCas13d.

TABLE 13

| | Cas13X1 | RfdCas13d | Cas13g3 |
|---|---|---|---|
| EZH2-spacer-1 | 0.313798 | 0.627438 | 0.306951 |
| EZH2-spacer-2 | 0.755891 | 0.408723 | 0.715392 |
| EZH2-spacer-3 | 0.89017 | 1.51347 | 0.539906 |
| NF2-spacer-1 | 0.324943 | 0.296356 | 0.381387 |
| NF2-spacer-2 | 0.666154 | 0.791354 | 0.533823 |
| NF2-spacer-3 | 0.817664 | 0.847641 | 0.383713 |
| HRAS-spacer-1 | 0.044778 | 0.024482 | 0.06682 |
| HRAS-spacer-2 | 0.446724 | 0.304201 | 0.425856 |
| HRAS-spacer-3 | 0.119186 | 0.169341 | 0.289796 |
| NRAS-spacer-1 | 0.393875 | 0.449508 | 0.51671 |
| NRAS-spacer-2 | 0.668483 | 0.333773 | 0.485217 |
| NRAS-spacer-3 | 0.584562 | 0.154314 | 0.564822 |
| PPARG-spacer-1 | 0.288989 | 0.259088 | 0.174556 |
| PPARG-spacer-2 | 0.107358 | 0.059001 | 0.077466 |
| PPARG-spacer-3 | 0.651076 | 0.57364 | 0.481814 |

Although the embodiments of the present invention have been described above in conjunction with the accompanying drawings, the present invention is not limited to the above-mentioned specific embodiments and application fields. The above-mentioned specific embodiments are only illustrative and instructive, rather than restrictive. Under the inspiration of this description and without departing from the scope of protection of the claims of the present invention, those of ordinary skill in the art can also make many forms, which are all included in the protection of the present invention.

1-15. (canceled)

16. An isolated Cas13 nuclease protein, the amino acid sequence of the Cas13 nuclease protein is:
a) the amino acid sequence shown in any one of SEQ ID NOs. 1 to 28; or
b) an amino acid sequence having at least 80% sequence identity with any one of SEQ ID NOs. 1 to 28 and having RNA cleavage activity.

17. The Cas13 nuclease protein of claim 16, wherein the Cas13 nuclease protein is Cas13bt1, Cas13bt2, Cas13g, Cas13h, Cas13i, Cas13j or Cas13k protein, preferably Cas13g3.

18. An engineered Cas13 nuclease effector protein, comprising a Cas13 nuclease protein, the Cas13 nuclease protein comprising:
a) the amino acid sequence shown in any one of SEQ ID NOs. 1 to 28; or,
b) an amino acid sequence having at least 80% sequence identity with any one of SEQ ID NOs. 1 to 28 and having RNA cleavage activity.

19. The effector protein of claim 18, wherein the Cas13 nuclease protein loses its catalytic activity through amino acid mutation, for example, the Cas13 nuclease protein forms dCas13 protein through amino acid mutations in the HEPN domain (RxxxxH motif) at its C-terminus and/or N-terminus.

20. The effector protein of claim 18, further comprising a functional domain fused to the Cas13 nuclease protein, the functional domain is selected from one or more of the following: translation initiation domain, translation repression domain, transactivation domain, epigenetic modification domain, nucleobase editing domain, reverse transcriptase domain, reporter domain and nuclease domain.

21. The effector protein of claim 20, wherein the nucleobase editing domain is adenosine deaminase, cytidine deaminase or their catalytic domains.

22. A polynucleotide, encoding the Cas 13 nuclease protein of claim 16.

23. A polynucleotide, encoding the effector protein of claim 18.

24. An engineered CRISPR-Cas13 gene editing system, comprising:
(a) the nuclease of claim 16, an engineered Cas13 nuclease effector protein, or the nucleic acid encoding the effector protein; and
(b) crRNA, comprising a spacer sequence complementary to a target sequence in a target nucleic acid;
wherein,
the engineered Cas13 nuclease effector protein, comprises a Cas13 nuclease protein, the Cas13 nuclease protein comprising:
a) the amino acid sequence shown in any one of SEQ ID NOs. 1 to 28; or,
b) an amino acid sequence having at least 80% sequence identity with any one of SEQ ID NOs. 1 to 28 and having RNA cleavage activity, and
the engineered Cas13 nuclease effector protein and the crRNA can form a CRISPR complex that specifically binds to a target nucleic acid comprising the target sequence and induces modification of the target nucleic acid.

25. An engineered CRISPR-Cas13 gene editing system, comprising:
(a) the engineered Cas13 nuclease effector protein of claim 18, or the nucleic acid encoding the effector protein; and
(b) crRNA, comprising a spacer sequence complementary to a target sequence in a target nucleic acid;
wherein, the engineered Cas13 nuclease effector protein and the crRNA can form a CRISPR complex that specifically binds to a target nucleic acid comprising the target sequence and induces modification of the target nucleic acid.

26. The engineered CRISPR-Cas13 gene editing system of claim 24, wherein the crRNA further comprises a direct repeat (DR) sequence, preferably the direct repeat (DR) sequence comprises any one of SEQ ID NOs. 29 to 56 or a sequence having at least 80% identity with the sequence shown in any one of SEQ ID NOs. 29 to 56.

27. The engineered CRISPR-Cas13 gene editing system of claim 25, wherein the crRNA further comprises a direct repeat (DR) sequence, preferably the direct repeat (DR) sequence comprises any one of SEQ ID NOs. 29 to 56 or a sequence having at least 80% identity with the sequence shown in any one of SEQ ID NOs. 29 to 56.

28. A method for modifying a cell comprising a target nucleic acid, comprising contacting the cell with the Cas13 nuclease protein of claim 16, thereby achieving modification of the target nucleic acid in the cell.

29. A method for modifying a cell comprising a target nucleic acid, comprising contacting the cell with the engineered Cas13 nuclease effector protein of claim 18, thereby achieving modification of the target nucleic acid in the cell.

30. A method for modifying a cell comprising a target nucleic acid, comprising contacting the cell with the engineered CRISPR-Cas13 gene editing system of claim 24, thereby achieving modification of the target nucleic acid in the cell.

31. A method for modifying a cell comprising a target nucleic acid, comprising contacting the cell with the engineered CRISPR-Cas13 gene editing system of claim 28, thereby achieving modification of the target nucleic acid in the cell.

32. A composition comprising the Cas13 nuclease protein of claim 16, for modification of the nucleic acids.

33. A composition comprising the engineered Cas13 nuclease effector protein of claim 18, for modification of the nucleic acids.

34. A composition comprising the engineered CRISPR-Cas13 gene editing system of claim 24, for modification of the nucleic acids.

35. A composition comprising the engineered CRISPR-Cas13 gene editing system of claim 25, for modification of the nucleic acids.
